(19) United States
(12) Patent Application Publication
(10) Pub. No.: US 2017/0340693 A1
(43) Pub. Date: Nov. 30, 2017

(54) DISLODGEMENT AND RELEASE OF HSC USING ALPHA 9 INTEGRIN ANTAGONIST AND CXCR4 ANTAGONIST

(71) Applicant: COMMONWEALTH SCIENTIFIC AND INDUSTRIAL RESEARCH ORGANISATION, Acton, ACT (AU)

(72) Inventors: Susan Kaye NILSSON, East Bentleigh, Victoria (AU); David Norman HAYLOCK, Melbourne, Victoria (AU); Benjamin Ben Ming CAO, Knoxfield, Victoria (AU)

(73) Assignee: COMMONWEALTH SCIENTIFIC AND INDUSTRIAL RESEARCH ORGANISATION, Acton, ACT (AU)

(21) Appl. No.: 15/535,144

(22) PCT Filed: Dec. 11, 2015
(86) PCT No.: PCT/AU2015/050783

§ 371 Date: Jun. 12, 2017

(30) Foreign Application Priority Data
Dec. 12, 2014 (AU) 2014905039

(51) Int. Cl. A61K 38/05; A61K 35/28; A61K 31/395

(52) U.S. Cl. CPC A61K 38/05; A61K 31/395; A61K 35/28

(57) ABSTRACT

Haematopoietic stem cell mobilization is a process whereby haematopoietic stem cells are stimulated out of the bone marrow space. Before HSC can mobilize, they must be dislodged and released from the BM stem cell niche in which they reside and are retained by adhesive interactions. Accordingly, in an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an α9 integrin or an active portion thereof and a CXCR4 antagonist or an active portion thereof to the BM stem cell niche. Once mobilized to the peripheral blood (PB) the HSC may be collected for transplant. Methods which enhance mobilization of the HSC can also improve treatments of haematological disorders.

### FIELD OF THE INVENTION

The present invention relates to enhancing dislodgement and release of haematopoietic stem cells (HSC) and precursors and progenitors thereof from a bone marrow (BM) stem cell niche and methods for enhancing the dislodgement and release of HSC and their precursors and progenitors thereof from the BM and the stem cell niche. The invention also relates to compositions for use in enhancing the dislodgement and release of HSC and their precursors and progenitors thereof. Cell populations of HSC and their precursors and progenitors thereof which have been dislodged and released by the methods and compositions are included as well as the use of the cell populations for treatment of a haematological disorders and transplantation of HSC, precursors and progenitors thereof.

### BACKGROUND OF THE INVENTION

HSC regulation and retention within the BM stem cell niche is mediated through interactions between HSC surface receptors and their respective ligands expressed by surrounding cells such as osteoblasts and sinusoidal endothelial cells. Spatial distribution analysis of HSC within BM using functional assays and in vivo and ex vivo imaging indicate they preferentially localize nearest the bone/BM interface within the endosteal niche. Of note, HSC identical to the classic Lin−Sca-1+ckit+CD150+CD48-phenotype, but isolated from endosteal BM have greater homing potential and enhanced long-term, multi-lineage haematopoietic reconstitution relative to HSC isolated from the central medullary cavity. Thus, the therapeutic targeting of endosteal HSC for mobilization should provide better transplant outcomes.

The localization of haematopoiesis to the BM involves developmentally regulated adhesive interactions between primitive haematopoietic cells and the stromal-cell-mediated haematopoietic microenvironment of the BM stem cell niche. Under steady-state conditions, HSC are retained in the BM niche by adhesive interactions with stromal elements (such as VCAM-1 and osteopontin (Opn)) leading to the physiologic retention of primitive haematopoietic progenitor cells in the BM. A perturbation of the adhesive interactions can lead to the release of the HSC retained in the BM and evoke the release of stem/progenitor cells from the bone marrow niche and eventually into the circulation by mobilization. The physiologic egress or mobilization of leukocytes from bone marrow ultimately to peripheral blood, as well as the escape of a small number of stem/progenitor cells from the normal bone marrow environment to the circulation, is a poorly understood phenomena. The movement of cells from the extravascular spaces of bone marrow to circulation may require a coordinated sequence of reversible adhesion and migration steps. The repertoire of adhesion molecules expressed by stem/progenitor cells or by stromal cells in bone marrow is crucial in this process. Alterations in the adhesion and/or migration of progenitor cells triggered by diverse stimuli would likely result in their dislodgment or redistribution between bone marrow and peripheral blood.

Releasing and mobilising specific populations of HSC may allow uses in various situations including transplantation, gene therapy, treatment of disease including cancers such as leukaemias, neoplastic cancers including breast cancers, or repair of tissues and skin. However, to mobilize HSC requires rapid and selective mobilization regimes which can initially dislodge the HSC from the BM. Dislodgement and release of specific cell populations of HSC from the BM stem cell niche can provide greater long-term, multi-lineage haematopoietic reconstitution.

The transplantation of mobilized peripheral blood (PB) haematopoietic stem cells (HSC) into patients undergoing treatment for blood diseases has essentially replaced traditional bone marrow (BM) transplants. Some clinical practices for HSC mobilization are achieved with a 5-day course of recombinant granulocyte-colony stimulating factor (G-CSF), which is believed to stimulate the production of proteases that cleave CXCR4/SDF-1 interactions. However, G-CSF is ineffective in a large cohort of patients and is associated with several side effects such as bone pain, spleen enlargement and on rare occasions, splenic rupture, myocardial infarction and cerebral ischemia.

These inherent disadvantages of G-CSF have driven efforts to identify alternate mobilization strategies based on small molecules. For example, the FDA-approved CXCR4 antagonist AMD3100 (Plerixafor; Mozobil™) has been shown to rapidly mobilize HSC with limited toxicity issues. Nevertheless, clinical mobilization with AMD3100 is only effective in combination with G-CSF and the search for rapid, selective and G-CSF independent mobilization regimens remains a topic of clinical interest. Although clinically G-CSF is the most extensively used mobilization agent, its drawbacks further include potentially toxic side effects, a relatively long course of treatment (5-7 days of consecutive injections), and variable responsiveness of patients.

However, to effect mobilization, the HSC must be released from their attachment to the BM stem cell niche. The molecules that are important in niche function and retaining the HSC in the niche environment include VCAM-1, Opn and Tenasin-C.

Integrins such as α₄β₁ have been implicated in the mobilization of HSC. Specifically both α₄β₁ (VLA-4) and α₉β₁ integrins expressed by HSC have been implicated in stem cell quiescence and niche retention through binding to VCAM-1 and Opn within the endosteal region. While the role of α₉β₁ integrin in HSC mobilization is unknown, the down-regulation of Opn using non-steroidal anti-inflammatory drugs (NSAID) as well as selective inhibition of integrin α₄ or G-CSF has validated Opn/VCAM-1 binding to integrins as effective targets for HSC mobilization. However, various characteristics such as binding to small molecules such as integrins show that they are distinctly different molecules.

In Pepinsky et al (2002) the difference between α₄β₁ and α₉β₁ integrins is evident in their binding characteristics. Pepinsky shows that the differences in binding to small molecule N-benzene-sulfonyl)-(L)-prolyl-(L)-O-(1-pyrrolidinyl carbonyl) tyrosine (BOP) is evident with EGTA treatment. The treatment inhibited binding of the monoclonal antibody 9EG7 to α₄β₁, whereas it stimulated the binding of 9EG7 to α₉β₁. Most notable was the estimated >1000 fold difference in the affinity of the integrins for VCAM-1 which binds α₄β₁ with an apparent K_{d} of 10 nM compared to α₉β₁ with an apparent K_{d} of >10 μM. Differences were also seen in the binding of α₉β₁ and α₄β₁ to Opn.

Accordingly, it is an aim of the present invention to provide compounds that facilitate the dislodgment and release of HSC, and thus improve mobilization of these cell types and treatment regimes that are independent of G-CSF. By providing these compounds that target specific HSC populations reconstitution and transplantation outcomes may be improved.

### SUMMARY OF THE INVENTION

In an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof and a CXCR4 antagonist or an active portion thereof to the BM stem cell niche in the presence or absence of G-CSF.

Preferably, the dislodgement of the HSC leads to release of the HSC from the BM stem cell binding ligand which enables the HSC to mobilize from the BM to the PB and thereby enhances mobilization of the HSC. Further stimulation of mobilization can be assisted by the use of mobilization agents that further enhance mobilization of HSC to the PB.

Preferably, the HSC are endosteal progenitor cells selected from the group including CD34⁺ cells, CD38⁺ cells, CD90⁺ cells, CD133⁺ cells, CD34⁺CD38⁻ cells, lineage-committed CD34⁻ cells, or CD34⁺CD38⁺ cells.

Preferably the antagonist of an α₉ integrin or an active portion thereof, is an α₉β₁ integrin or an active portion thereof.

In another embodiment, the method further includes administering an antagonist of α₄ integrin or an active portion thereof. Preferably the α₄ integrin is an antagonist of α₄β₁ or an active portion thereof.

In another embodiment, the antagonist cross-reacts with α₉ and α₄, and optionally cross-reacts with α₉β₁ and α₄β₁. Optionally, the antagonist is a α₉β₁/α₄β₁ antagonist or an active portion thereof.

Preferably, the antagonist is a compound of Formula (I) or a pharmaceutically acceptable salt thereof having the formula:

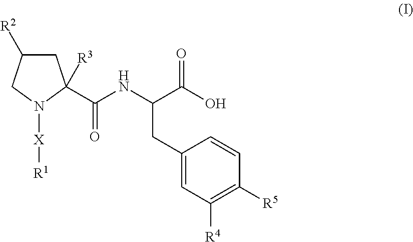

wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

Preferably, the compound of Formula (I) has the following Formula (Ia)

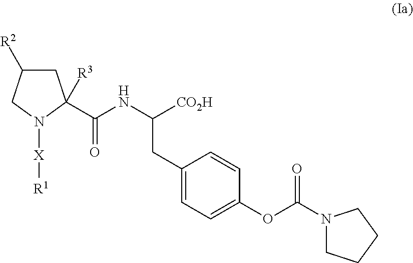

or a pharmaceutically acceptable salt thereof.

In one embodiment, the compound of Formula (I) has the following Formula (Ib)

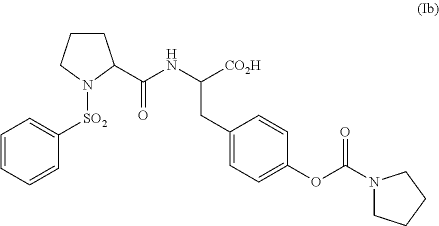 [SMILES: O=C(O)C(Cc1ccc(OC(=O)N2CCCC2)cc1)NC(=O)C1CCCN1S(=O)(=O)c1ccccc1]

or a pharmaceutically acceptable salt thereof.

Preferably, the compound of Formula (I) has the following Formula (Ic)

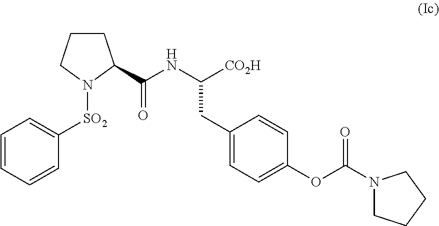 [SMILES: O=C(O)[C@H](Cc1ccc(OC(=O)N2CCCC2)cc1)NC(=O)[C@@H]1CCCN1S(=O)(=O)c1ccccc1]

or a pharmaceutically acceptable salt thereof.

In another embodiment, the compound of Formula (I) has the following Formula (Id)

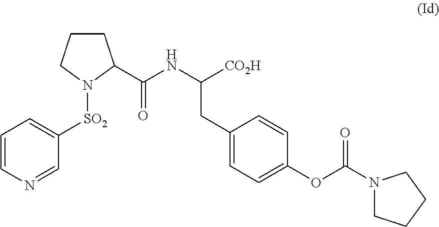 [SMILES: O=C(O)C(Cc1ccc(OC(=O)N2CCCC2)cc1)NC(=O)C1CCCN1S(=O)(=O)c1cccnc1]

or a pharmaceutically acceptable salt thereof.

Preferably, the compound of the above Formula has the following Formula (Ie)

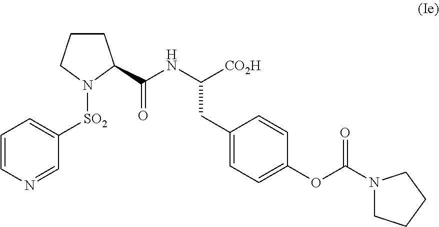 [SMILES: O=C(O)[C@H](Cc1ccc(OC(=O)N2CCCC2)cc1)NC(=O)[C@@H]1CCCN1S(=O)(=O)c1cccnc1]

or a pharmaceutically acceptable salt thereof.

In another embodiment there is provided a composition for enhancing dislodgement, release or mobilization of HSC from a BM stem cell binding ligand said composition comprising an antagonist of α₉ integrin or an active portion thereof and a CXCR4 antagonist or an active portion thereof as herein described.

In yet another aspect of the invention, there is provided a method of harvesting HSC from a subject said method comprising:
administering in the presence or absence of G-CSF an effective amount of an antagonist of as integrin or an active portion thereof and a CXCR4 antagonist or an active portion thereof to a subject wherein said effective amount enhances dislodgement of HSC and their precursors and progenitors thereof from a BM stem cell binding ligand in a BM stem cell niche;
mobilizing the dislodged HSC to PB; and harvesting the HSC from the PB.

In even further aspects of the invention methods are provided for the treatment of a haematological disorder in a subject said method comprising administering to the subject in the presence or absence of G-CSF, a therapeutically effective amount of an antagonist of α₉ integrin or an active portion thereof as herein described and a CXCR4 antagonist or an active portion thereof or a cell composition comprising HSC harvested from a subject administered with the antagonist of α₉ integrin or an active portion thereof and a CXCR4 antagonist or an active portion thereof as herein described to enhance dislodgement, release or mobilization of HSC from the BM to the PB.

In yet another preferred embodiment, the haematological disorder is a haematopoietic neoplastic disorder and the method involves chemosensitizing the HSC to alter susceptibility of the HSC, such that a chemotherapeutic agent, having become ineffective, becomes more effective.

In yet another aspect there is provided a method of transplanting HSC into a patient, said method comprising
administering an as integrin antagonist or an active portion thereof and a CXCR4 antagonist or an active portion thereof to a subject to dislodge HSC from a BM stem cell binding ligand;
releasing and mobilizing the HSC from the BM to the PB;
harvesting HSC from the PB from the subject; and
transplanting the HSC to the patient.

Other aspects of the present invention will become apparent to those ordinarily skilled in the art upon review of the following description of specific embodiments of the invention.

### FIGURES

For a further understanding of the aspects and advantages of the present invention, reference should be made to the following detailed description, taken in conjunction with the accompanying drawings.

FIG. 1 shows (a) R-BC154 (IXb) specifically binds murine α₄ and α₉ expressed by CHO cells in the presence of divalent metal cations (Ca²⁺/Mg²⁺) (black solid line). R-BC154 (IXb) binding is abrogated in the presence of EDTA (black dotted line) (b) Use of R-BC154 (IXb), BOP and BIO5192 to demonstrate specific detection of binding to α₉ (grey) in human and (c) murine compared to α₄ (black) in the presence of 1 mM Ca²⁺/Mg²⁺. n=3 and is representative of at least 2 independent experiments.

FIG. 2 shows (a) Dose response of R-BC154 (IXb) binding to human CB MNC. n=3 and is representative of 2 independent experiments. (b) Representative populations of CB HSC (CD34⁺CD38⁻), progenitors (CD34⁺CD38⁺) and committed cells (CD34⁻CD38⁺). (c) R-BC154 (IXb) binding to CB MNC committed cells, progenitors and HSC in the presence of 1 mM Ca²⁺/Mg²⁺ alone (solid black line), or in combination with BOP (solid grey line) or BIO5192 (dotted black line). Data is representative of 3 individual samples. α₉-specific binding via α₉β₁, α₄-specific binding via α₄β₁ (d) Specific binding of R-BC154 (IXb) binding to α₉β₁ on CB committed cells, progenitors and HSC. n=3 UD—undetectable (e) R-BC154 (IXb) binding to human BM (huBM). Data is representative of 3 individual samples. (f) Specific binding of R-BC154 (IXb) to huBM α₉β₁, n=3 one way ANOVA p<0.01 (g) α₉β₁ expression on huBM progenitors and HSC (black solid line, dashed line IgG1 isotype control). Data is representative of 3 individual samples (h) Generation of humanized NODSCIDIL2Rγ−/− (huNSG) mice from CB mononuclear cells (MNC) and flow cytometric analysis of BM white blood cell (WBC) CD34⁺CD38⁺ progenitors, CD34⁺CD38⁻ HSC and CD34⁻CD38⁺ committed cells. (i) R-BC154 (IXb) binding to humanized NSG (huNSG) BM. Data is representative of 3 individual samples. (j) Specific binding of R-BC154 (IXb) to huNSG BM α₉β₁. n=3 one way ANOVA p<0.01 (k) α₉β₁ expression on huNSG BM progenitors and HSC (black solid line, dashed line IgG1 isotype control). Data is representative of 3 individual samples (I) Expression of α₄ (solid line) on huNSG BM committed cells, progenitors and HSC. Isotype=dashed line. (m) Quantified data from FIG. 2i depicting R-BC154 (IXb) binding in the presence of Ca²⁺/Mg²⁺ to huNSG BM committed cells, progenitors and HSC. n=3. one way ANOVA p<0.005

FIG. 3 shows (a) Representative flow cytometry plot of murine BM Lin⁻Sca⁺c-kit⁺ (LSK; progenitors) and LSKCD150⁺CD48⁻ (LSKSLAM; HSC). (b) R-BC154 (IXb) binding to murine progenitors and HSC in the presence of 1 mM Ca²⁺/Mg²⁺ (black bar) or 10 mM EDTA (c) Schematic of a femur depicting endosteal (eBM) and central (cBM) BM and in vitro R-BC154 (IXb) binding to central (cBM) and endosteal (eBM) progenitor cells and HSC in the absence of exogenously added cations. (d) Comparative R-BC154 (IXb) binding to lymphoid (B220⁺ and CD3⁺) and myeloid (Gr1Mac1), LSK and LSKSLAM cells from central and endosteal BM in the presence of 1 mM Ca²⁺/Mg²⁺. Data is representative of 2 separate experiments. One-way ANOVA p<0.0001 (e) R-BC154 (IXb) binding to central and endosteal LSK and LSKSLAM cells from wt (black bar) and α₄^{-/-}/α₉^{-/-} conditional KO mice (white bar) t-test p<0.01.

FIG. 4 shows plots of gated lymphoid (B220⁺ and CD3⁺), myeloid (Gr1Mac1⁺) and lineage⁻ populations from endosteal and central WBM treated with R-BC154 (IXb) (10 nM) in the presence of 10 mM EDTA (grey line) and 1 mM Ca²⁺/Mg² (black line). Data is a representative of n=3.

FIG. 5 shows the α₉β₁/α₄β₁. integrin antagonist BOP rapidly mobilizes murine long-term repopulating HSC. (a) Dose-dependent mobilization of murine progenitors (LSK, white square) and HSC (LSKSLAM, grey square) with BOP. Data is pooled from two biological repeats; n>3 per group. One-way ANOVA p<0.05 (b) Time-course of progenitor and HSC mobilization with 10 mg/kg BOP. Data is pooled from two biological repeats. n=5 individual animals per time point, not repetitive analysis. Time 0 is the vehicle control. One-way ANOVA p<0.05

FIG. 6 shows (a) Extended time-course analysis of total progenitors (LSK), (b) HSC (LSKSLAM) and (c) lymphocyte content in PB for up to 18 hours after a single dose of BOP. n=5. One-way ANOVA p<0.05 (d) Analysis of LSK and LSKSLAM content in PB of mice treated with saline or R-BC154 (IXb) over 30 minutes. Data is pooled from two independent experiments. n>6 (e) Analysis of in vivo BOP binding to endosteal versus central progenitors (LSK) and HSC (LSKSLAM) following the administration of BOP alone or BOP in combination with AMD3100. Data is presented as fold increase in BOP binding to endosteal cells relative to their central counterparts (dotted line). n=3. p-values represent statistically significant results using paired t-test between central and endosteal populations within each treatment group. P<0.05

FIG. 7 shows plots that represent a comparison of in vitro staining of R-BC154 (IXb) in the presence of exogenous Ca²⁺/Mg²⁺ (solid black line) and in vivo administration of R-BC154 (IXb) versus R-BC154 (IXb) in the presence of exogenous Ca²⁺/Mg²⁺+BOP (dotted line). Data is a representative of n=3.

FIG. 8 shows enhanced HSC mobilization using BOP in combination with AMD3100. (a) Representative flow cytometry plots and (b) determination of the LSK content (c) LPP-CFC content and (d) HPP-CFC content in peripheral blood of mice treated with saline (n=3), BOP (n=4), AMD3100 (n=4) and the combination of BOP and AMD3100 (n=4). Data is mean±SEM. (e) Analysis of WBC content in peripheral blood of mice treated with saline, BOP (10 mg/kg) and AMD3100 (3 mg/kg) alone and in combination with BOP (at 1, 5 and 10 mg/kg). Data is mean±SEM. One-way ANOVA p<0.05 (f) Limiting dilution transplant analysis of RFP blood mobilized by BOP, AMD3100 and the combination of BOP and AMD3100 (5 recipients per group). Positive multi-lineage engraftment was considered as >0.5% CD3⁺, B220⁺ and Gr1Mac1⁺. (g) survival of recipients transplanted with 30 μl of RFP mobilized PB. (h) Long-term HSC frequency in PB mobilized following BOP, AMD3100 or BOP+AMD3100. All data is mean±SEM One-way ANOVA *p<0.05, **p<0.01, ***p<0.005, ****p<0.001.

FIG. 9 shows (a) PB white blood cell (WBC) content 1 hour after a single dose of vehicle (n=5), BIO5192 alone (n=5) or in combination with AMD3100 (n=5), or BOP in combination with AMD3100 (n=3). (b) Total PB progenitor (LSK) and HSC (LSKSLAM) content 1 hour after a single dose of vehicle (n=5), BIO5192 alone (n=5) or in combination with AMD3100 (n=5), or BOP in combination with AMD3100 (n=3). All data is mean±SEM. One-way ANOVA *p<0.05, **p<0.01, ***p<0.005, ****p<0.001

FIG. 10 shows (a) Analysis of PB progenitor (LSK) and HSC (LSKSLAM) content following a single dose of BOP+AMD3100 or 4 days of G-CSF. Data is a pool of 2 biological repeats. n>8. (b) Schematic of serial competitive transplant assay. (c) Proportion of PB RFP+ white blood cells (WBC) in 1° recipients. Each data point is an individual recipient from one of two transplants. 1st=crosses, 2nd=circles. Dashed line is mathematically expected engraftment level. (d) Analysis of PB RFP+ and GFP+lymphoid (B220+ and CD3+) and myeloid (Gr1+/Mac1+) engraftment in 1° recipients, n=8; pooled from 1st and 2nd experiments. (e) Analysis of donor engraftment and (f) lineage distribution (lymphoid, B220+ and CD3+ and myeloid, Gr1+/Mac1+) in BM of 1° recipients. (g) 2° recipient PB analysis. Each data point is an individual recipient. Dashed line is mathematically expected engraftment level. (h) Analysis of PB RFP+ and GFP+lymphoid (B220+ and CD3+) and myeloid (Gr1+/Mac1+) engraftment in 2° recipients 20 weeks post-transplant. Data is grouped based on the 5 individual 20 donors. n=4 2° recipients per donor. UD=undetectable. (i) Analysis of donor engraftment and (j) lineage distribution (lymphoid, B220+ and CD3+ and myeloid, Gr1+/Mac1+) in BM of 2° recipients 20 weeks post-transplant. All symbols are individual animals and all data is mean±SEM. *p<0.05, **p<0.01, ***p<0.005, ****p<0.001.

FIG. 11 shows the combination of BOP and AMD3100 effectively mobilizes human CD34+ stem and progenitors in humanized NSG (huNSG) mice. The Plot shows the analysis of huCD45+CD34+ cell content in huNSG PB following mobilization. Data is expressed as fold-increase in CD34+ cells/ml PB relative to saline control and each data point represents an individual animal. Black bar=mean. **p<0.01, ****p<0.001.

FIG. 12 shows an alternate small molecule to BOP that is of similar structure but the benzene sulfonyl group has been replaced with a 3-pyridinesulfonyl group. Data shows no compounds, Py-Bop alone, AMD3100 alone and Py-BOP and AMD3100 for (a) progenitors (LSK) and (b) HSC (LSKSLAM). Data is mean±SEM. *p<0.05, **p<0.01.

FIG. 13 shows (a) ALL cells mobilized to PB, represented as fold increase. (b) The addition of AMD3100 to BOP increases the % of α₄β₁/α₉β₁ occupied by BOP. Data is mean±SEM. T-test *p<0.05.

### DETAILED DESCRIPTION OF THE INVENTION

Haematopoietic stem cell mobilization is a process whereby haematopoietic stem cells are stimulated out of the bone marrow space (e.g., the hip bones and the chest bone) into the bloodstream, so they are available for collection for future reinfusion or they naturally egress from the bone marrow to move throughout the body to lodge in organs such as the spleen to provide blood cells. This interesting natural phenomenon, that often accompanies various haematological disorders, may be adapted as a useful component of therapy, given the discovery of agents that can artificially incite mobilization of HSCs into the bloodstream where they can be collected and used for purposes such as transplantation. Compounds such as G-CSF and the FDA-approved CXCR-4 antagonist AMD 3100 have been shown to mobilize HSC. However, toxicity issues and various side effects can result from this treatment.

Before HSC can mobilize, they must be dislodged and released from the BM stem cell niche in which they reside and are retained by adhesive interactions.

Accordingly, in an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof and a CXCR4 antagonist or an active derivative thereof to the BM stem cell niche.

In steady state conditions, HSC reside in the BM in specialized locations called the BM stem cell niche. Here they reside as quiescent stem cells before they are released ready to enter the PB and lodge in tissues to start differentiating. The HSC are retained in the BM stem cell niche by adhesion molecules or binding ligands such as but not limited to VCAM-1, Opn and Tenacin-C. Management of the HSC/BM stem cell niche interaction is instrumental in the dislodgement and release of HSC to the BM stem cell niche and eventually to the PB.

Hence the present invention provides a means to dislodge and release the HSC from the interactions in the BM stem cell niche by disrupting the adhesive interactions and binding ligands between the HSC and the BM stem cell niche environment. The cells then become available for mobilizing to the PB or they may remain in the BM.

The BM stem cell niche includes the endosteal niche and the central medullary cavity. The endosteal stem cell niche is located at the endosteum of the bone marrow, where osteoblasts are the main regulators of HSC functions such as proliferation and quiescence. Furthermore, a significant proportion of HSC are closely associated with sinusoidal endothelial cells in the endothelial niche where they are ready to enter peripheral blood and start differentiation. The central medullary cavity is the central cavity of the bone responsible for the formation of red blood cells and white blood cells otherwise known as the bone marrow.

Applicants have found that by inhibiting at least the α₉ integrin with small molecule antagonists in the presence of a CXCR4 antagonist or an active portion thereof, HSC and their precursors and progenitors thereof can dislodge from the BM stem cell niche preferably into the endosteal niche or mobilize into the PB with long term multi-lineage engraftment potential. Surprisingly it has been found that the use of an antagonist to α₉ integrin or an active portion thereof and a CXCR4 antagonist or an active derivative thereof significantly increases the dislodgement and release of CD34⁺ stem cells and progenitors into the blood.

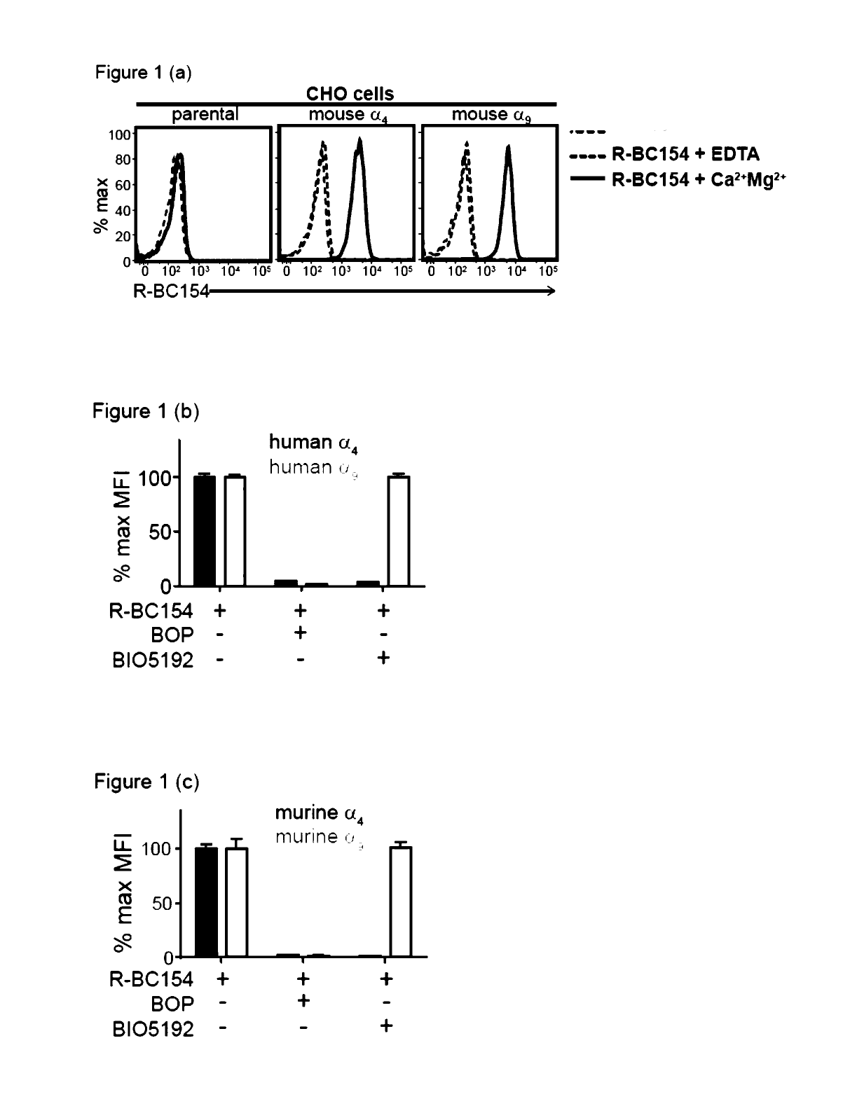

Applicants have developed a fluorescent small molecule integrin antagonist, R-BC154 (IXb) (1) (FIG. 1a), based on a series of N-phenylsulfonylproline dipeptides, which bind activated human and murine α₉β₁ and α₄β₁ integrins as well as BM HSC and progenitors (FIG. 1a). Applicants postulated that this family of compounds would target potent endosteal HSC for mobilization based on the restricted interaction between α₉β₁/α₄β₁ and Opn within endosteal BM. It has now been found that R-BC154 (IXb) (1) and its non-labelled derivative BOP (2) preferentially bind and mobilize mouse and human HSC and progenitors via intrinsically activated α₉β₁/α₄β₁ integrins in vivo. In addition, when BOP is used in combination with AMD3100 a CXCR4 antagonist, greater mobilization of long-term repopulating HSC was achieved in 1 h relative to a 4-day G-CSF regimen. The combination of BOP and AMD3100 was also found to mobilize human CD34⁺ cells in a humanised NODSCIDIL2Rγ^{-/-} mouse model. Thus, therapeutic targeting of endosteal HSC using as α₉β₁/α₄β₁ integrin inhibitors either alone or in combination with AMD3100 offers a promising alternative to current mobilization strategies for stem cell transplant applications.

Integrins are non-covalently linked αβ heterodimeric trans-membrane proteins that function primarily as mediators of cell adhesion and cell signalling processes. They are composed of an alpha chain and a beta chain, each chain playing different roles and having different metal binding sites important for their activation and activity. In mammals, 18 α-chains and 8 β-chains have been identified, with 24 different and unique αβ combinations described to date.

The α₄β₁ integrin (very late antigen-4; VLA-4) is expressed primarily on leukocytes and is known to be a receptor for vascular cell adhesion molecule-1 (VCAM-1), fibronectin and Opn. The α₄β₁ integrin is a key regulator of leukocyte recruitment, migration and activation and has important roles in inflammation and autoimmune disease. Accordingly, significant effort has been focused on the development of small molecule inhibitors of α₄β₁ integrin function for the treatment of asthma, multiple sclerosis and Crohn's disease, with several candidates progressing to phase I and II clinical trials.

Integrin α₉, a structurally similar integrin protein, encoded by the ITGA9 gene has also been studied recently by Pepinsky et al (2002). The α₉ subunit forms a heterodimeric complex with a β₃ subunit to form the α₉β₁ integrin.

Whilst this related integrin, α₉β₁, shares many of the structural and functional properties as α₄β₁ there are differences between the integrins α₄β₁ and α₉β₁ which make them distinct. Unlike α₄β₁ which has a restricted expression that is largely on leukocytes, the cellular expression of α₉β₁ is widespread.

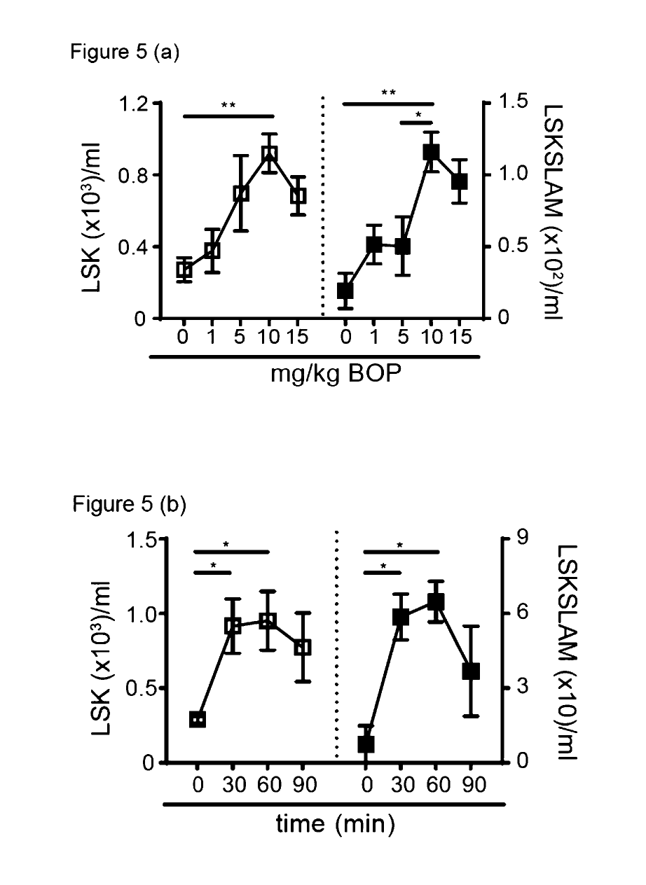

Furthermore, whilst binding to several of the same ligands including VCAM-1 and Opn, binding of other small molecules to α₉β₁ and α₄β₁ integrins have been shown to be different. As shown in the Examples herein, the greatest difference is in the off-rate kinetics. An α₉β₁ antagonist (R-BC154 (IXb)) as well as BOP are shown to have significantly reduced off-rates for α₉β₁ compared to α₄β₁. The details for R-BC154 (IXb) is exemplified in Example 2 herein (FIG. 5c) and details for BOP are exemplified in Pepinsky et al (2002).

Previously, both α₄β₃ and α₉β₁ integrins have been shown to be expressed by haemopoietic stem cells (HSC). The integrins α₄β₁ and α₉β₁ are primarily involved in the sequestration and recruitment of HSC to the bone marrow as well as the maintenance of HSC quiescence, a key characteristic for long-term repopulating stem cells.

HSC regulation by α₄β₁ and α₉β₁ integrins is mediated through interactions with VCAM-1 and Opn, which are expressed and/or secreted by bone-lining osteoblasts, endothelial cells and other cells of the bone marrow environment. However, as discussed in Pepinsky et al (2002) the difference in binding affinity for VCAM-1 and Opn are markedly different between α₄β₁ and α₉β₁. Small molecule inhibitors of α₄β₁ have been implicated as effective HSC mobilization agents. However, despite the structural and functional similarities between α₄β₁ and α₉β₁, the binding characteristics are different and hence the role of α₉β₁ integrin in this regard remains unexplored.

In one preferred embodiment of the invention, the antagonist of α₉ integrin is an antagonist of the α₉β₁ integrin. Accordingly, it is preferred that the antagonist of α₉ integrin is an antagonist of the α₉β₁ integrin or an active portion thereof.

As used herein, an active portion of the α₉β₁ integrin or of the α₄β₁ integrin is a portion of the α₉β₁ protein or α₄β₁ protein which retains activity of the integrin. That is, the portion is a part of the α₉β₁ protein or the α₄β₁ a43 protein which is less than the complete protein, but which can still act in the same or similar manner as the full α₉β₁ or α₄β₁ protein. Where the term “α₉ integrin” or “α₄ integrin” or “α₉β₁ integrin” or “α₄β₁ integrin” is used herein, it also includes reference to any active portions thereof.

Similarly, as used herein, an active derivative of the CXCR4 antagonist is a compound that is similar to the CXCR4 antagonist which retains activity of the CXCR4 antagonist. Where the term “CXCR4 antagonist” is used herein, it also includes reference to the active derivatives thereof.

In another embodiment of the present invention, the antagonist of α₉ integrin, preferably the α₉β₁ integrin is also an antagonist of α₄ integrin, preferably the α₄β₁ integrin. It is desired that the α₉ integrin antagonist of the present invention can inhibit the activity of both the α₉β₁ integrin and α₄β₁ integrin. Hence it is preferred that the antagonist is an α₉β₁/α₄β₁ integrin antagonist. In other words it is preferred that the antagonist reacts with α₄β₁ as well as α₉β₁, that is cross reacts with both integrins.

The antagonist of the α₉ integrin, preferably the α₉β₁ integrin may be the same or different to the antagonist of the α₄ integrin preferably the α₄β₁ integrin. If the antagonist is the same, a single antagonist may be used to inhibit the activity of both the α₉ integrin and the α₄ integrin. Separate antagonists may be used either simultaneously or sequentially to inhibit the α₉ integrin, preferably the α₉β₁ integrin and the α₄ integrin, preferably the α₄β₁ integrin.

In yet another embodiment of the invention it is preferred that the α₉ integrin, preferably the α₉β₁ integrin and the α₄ integrin preferably the α₄β₁ integrin are activated prior to the interaction of the integrin antagonist. The antagonist preferably interacts with intrinsically activated integrins. Therefore, it is desirable that the as integrin is intrinsically activated. Preferably, the α₉β₁ integrin is intrinsically activated. As contemplated above, it is desirable that both the α₉β₁ integrin/α₄β₁ integrin are activated simultaneously or sequentially so that the integrin antagonist targets the HSC and progenitors via intrinsically activated α₉/α₄ integrins in the endosteal niche.

Integrin activation is an important mechanism through which cells regulate integrin function by manipulating the ligand affinity of integrins spatially and temporally. The integrins can be activated from the inside by the regulated binding of proteins or from the outside by multivalent ligand binding. Ligand binding to external domains cause conformational changes that increase ligand affinity, modify protein-interaction sites in the cytoplasmic domains and the resulting signals. Thus, activation of the integrins may be achieved intrinsically or by use of divalent cations

In another embodiment of the present invention, the antagonist of an α₉ integrin, preferably the antagonist of α₉β₁ integrin, more preferably an α₉β₁/α₄β₁ integrin comprises a compound of Formula (I) or a pharmaceutically acceptable salt thereof having the formula:

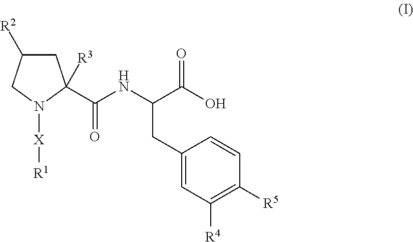

wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In one set of embodiments of the compound of Formula (I):
R⁴ is H;
R⁵ is —OR⁷;
   and X, R¹, R², R³ and R⁷ are as defined in Formula (I).

In such embodiments, the compound of Formula (I) may have a structure of Formula (II):

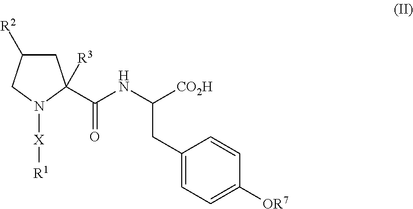

wherein:
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In one set of embodiments of a compound of Formula (I) or Formula (II):
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n is 1 or 2.

In some embodiments of a compound of Formula (I) or Formula (II), R⁷ is selected from C₁-C₄ alkyl.

Exemplary C₁-C₄ alkyl as described herein for groups of Formula (I) or Formula (II) may be linear or branched. In some embodiments, C₁-C₄ alkyl may be selected from the group consisting of methyl, ethyl, n-propyl, iso-propyl, n-butyl, sec-butyl, iso-butyl and tert-butyl.

In some embodiments, R⁷ may be methyl or tert-butyl, such that —OR⁷ is —OCH₃ or —OC(CH₃)₃.

In some embodiments of a compound of Formula (I) or Formula (II), R⁷ is —(CH₂)ₙ—R¹². In such embodiments, R¹² may be selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN, and n is an integer in the range of from 1 to 3.

In some embodiments of a compound of Formula (I) or Formula (II), R⁷ is —(CH₂)ₙ—R¹², where R¹² may be selected from the group consisting of —CN, —O(C₁-C₄ alkyl) and optionally substituted heteroaryl, and n is 1 or 2.

In some embodiments of a compound of Formula (I) or Formula (II), R⁷ is —(CH₂)ₙ—R¹², where:
R¹² is —OCH₃ and n is 2, or
R¹² is an optionally substituted tetrazolyl (preferably 5-tetrazolyl), and n is 1.

In some embodiments of a compound of Formula (I) or Formula (II), R⁷ is —C(O)R¹³. In such embodiments, R¹³ may be selected from the group consisting of optionally substituted cycloalkyl, optionally substituted aryl and optionally substituted heteroaryl.

In one set of embodiments, R¹³ may be an optionally substituted 5- or 6-membered cycloalkyl ring. Exemplary cycloalkyl rings may be cyclopentyl or cyclohexyl.

In one set of embodiments, R¹³ may be an optionally substituted aryl ring. An exemplary aryl ring is phenyl.

In one set of embodiments, R¹³ may be an optionally substituted heteroaryl ring. An exemplary heteroaryl ring is pyrrolyl.

In some embodiments of a compound of Formula (I) or Formula (II), R⁷ is —C(O)NR¹⁴R¹⁵.

In some embodiments of a compound of Formula (I) or Formula (II) where R⁷ is —C(O)NR¹⁴R¹⁵, R¹⁴ and R¹⁵ may each be independently selected from the group consisting of C₁-C₄ alkyl and optionally substituted aryl.

In some specific embodiments of a compound of Formula (I) or Formula (II) where R⁷ is —C(O)NR¹⁴R¹⁵, R¹⁴ and R¹⁵ are each ethyl or iso-propyl.

In one specific embodiment of a compound of Formula (I) or Formula (II) where R⁷ is —C(O)NR¹⁴R¹⁵, one of R¹⁴ and R¹⁵ is methyl and the other of R¹⁴ and R¹⁵ is phenyl.

In some embodiments of a compound of Formula (I) or Formula (II) where R⁷ is —C(O)NR¹⁴R¹⁵, R¹⁴ and R¹⁵, together with the nitrogen to which they are attached, may form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In specific embodiments of a compound of Formula (I) or Formula (II) R⁷ is —C(O)NR¹⁴R¹⁵, where R¹⁴ and R¹⁵, together with the nitrogen to which they are attached, form an optionally substituted pyrrolidinyl ring.

In some specific embodiments of a compound of Formula (I) or Formula (II):
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

In one set of embodiments of a compound of Formula (I), X is —SO₂—. In such embodiments, the compound of Formula (I) may have a structure of Formula (III):

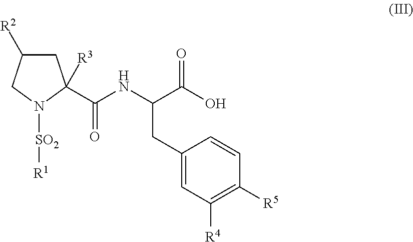

wherein:
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (III), R⁴ is H and R⁵ is OR⁷ to provide a compound of Formula (IIIa):

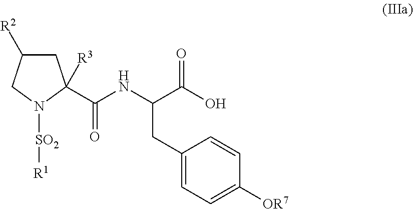

wherein
R¹, R² and R³ are as defined in Formula (III);
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of Formula (IIIa), R⁷ is selected from the group consisting of C₁-C₄ alkyl (preferably methyl or tert-butyl), —(CH₂)ₙ—R¹², —C(O)R¹³ and —C(O)NR¹⁴R¹⁵; wherein
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n is an integer selected from the group consisting of 1, 2 and 3.

In specific embodiments of Formula (IIIa), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In a specific embodiment of Formula (I), X is —SO₂—, R⁴ is H and R⁵ is —OR⁷, where R⁷ is —C(O)NR¹⁴R¹⁵ and R¹⁴ and R¹⁵, together with the nitrogen to which they are attached, form a pyrrolidinyl ring. In such embodiments, the compound of Formula (I) may have a structure of Formula (IIIb):

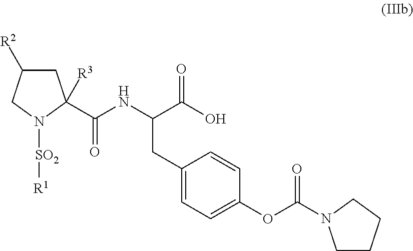

wherein R¹, R² and R³ are as defined herein.

In one set of embodiments of a compound of Formulae (I), (II), (III), (IIIa) or (IIIb) described herein, R¹ is an optionally substituted aryl. In another embodiment, R¹ is an optionally substituted heteroaryl. In some embodiments R¹ is an optionally substituted phenyl. In another embodiment R¹ is an optionally substituted pyridyl.

In one set of embodiments, R¹ is phenyl substituted with at least one halogen group. Halogen substituent groups may be selected from the group consisting of chloro, fluoro, bromo or iodo, preferably chloro.

In some embodiments, R¹ is phenyl substituted with a plurality of halogen groups. The halogen substituent groups may be positioned at the 3- and 5-positions of the phenyl ring. In another set of embodiments, R¹ is pyridyl. In such an embodiment the rest of the molecule may be positioned meta to the pyridyl nitrogen atom.

In one embodiment, a compound of Formula (I) may have a structure of Formula (IVa), (IVb) or (IVc):

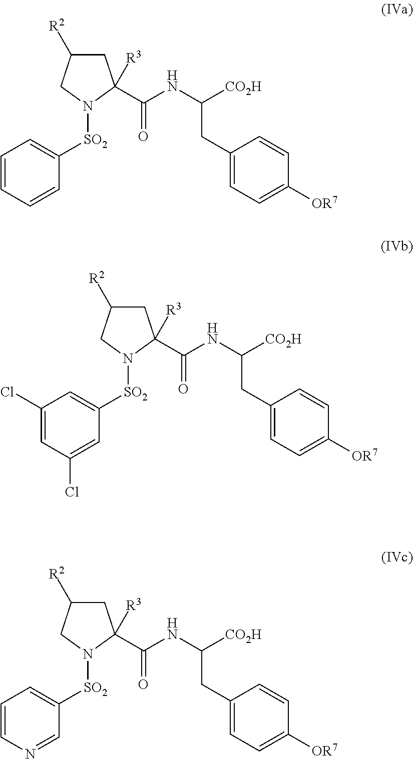

wherein in each of (IVa), (IVb) and (IVc), R², R³ and R⁷ are as defined in Formula (I).

In one set of embodiments of a compound of Formula (IVa), (IVb) or (IVc):
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CON
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (I), (II), (III), (IIIa), (IIIb), (IVa), (IVb), or (IVc) as described herein, R³ is H.

In embodiments where R³ is H, the compound of Formula (I) may have a structure of Formula (V):

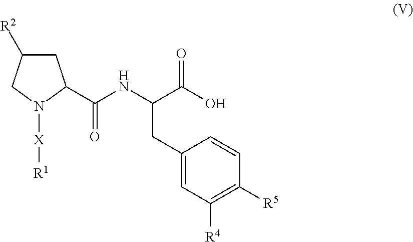

wherein:
X is selected from the group consisting of a bond and —SO₂—;
R¹ is selected from the group consisting of H, alkyl, optionally substituted aryl and optionally substituted heteroaryl;
R² is selected from the group consisting of H and a substituent group; R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (V), R⁴ is H and R⁵ is OR⁷ to provide a compound of Formula (Va):

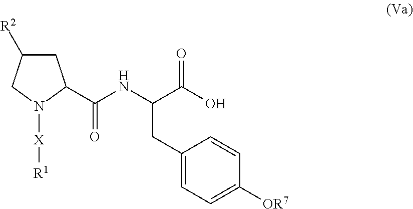

wherein
X, R¹, R² and R⁷ are as defined in Formula (V).

In some embodiments of a compound of Formula (Va), R⁷ is selected from the group consisting of C₁-C₄ alkyl (preferably methyl or tert-butyl), —(CH₂)ₙ—R¹², —C(O)R¹³ and —C(O)NR¹⁴R¹⁵; wherein R¹², R¹³, R¹⁴, R¹⁵ and n are as defined herein for Formula (V).

In specific embodiments of a compound of Formula (Va), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In some embodiments of a compound of Formula (V) or (Va), X is —SO₂—.

In a specific embodiment of a compound of Formula (Va), X is —SO₂—, R³ and R⁴ are each H and R⁵ is —OR⁷, where R⁷ is —C(O)NR¹⁴R¹⁵ and R¹⁴ and R¹⁵, together with the nitrogen to which they are attached, form a pyrrolidinyl ring. In such embodiments, the compound of Formula (V) may have a structure of Formula (Vb):

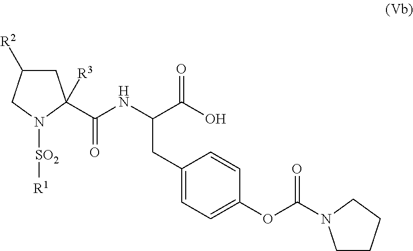

In one set of embodiments of a compound of Formula (V) or (Va), R¹ is an optionally substituted aryl, preferably an optionally substituted phenyl. The optional substituent is preferably at least one halogen group selected from the group consisting of chloro, fluoro, bromo or iodo, preferably chloro.

In one set of embodiments, R¹ is phenyl substituted with at least one halogen group. In some embodiments, R¹ is phenyl substituted with a plurality of halogen groups. The halogen substituent groups are preferably positioned at the 3- and 5-positions of the phenyl ring.

In one embodiment, a compound of Formula (V) may have a structure of Formula (VIa), (VIb) or (VIc):

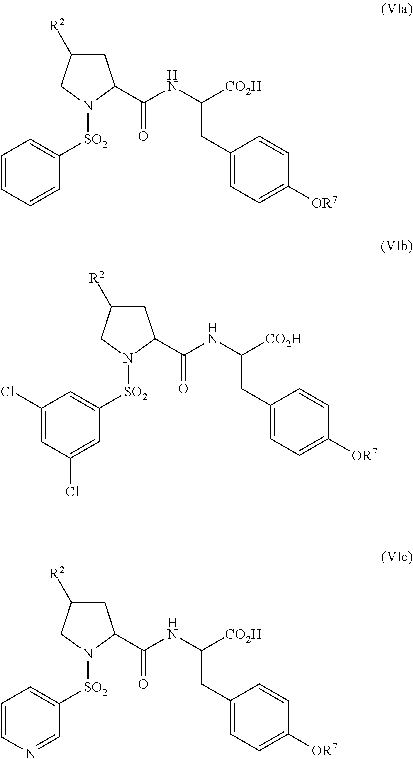

wherein in each of (VIa) and (VIb), R² and R⁷ are as defined in Formula (V).

In one set of embodiments of a compound of Formula (VIa), (VIb) or (VIc):
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In another set of embodiments of a compound of Formula (VIa), (VIb) or (VIc), R⁷ is selected from the group consisting of methyl, tert-butyl, —(CH₂)ₙ—R¹² where R¹² is selected from the group consisting of —CN, —CH₃, —C(CH₃)₃ and optionally substituted heteroaryl (preferably 5-tetrazolyl), and n is 1 or 2.

In another set of embodiments of a compound of Formula (VIa), (VIb) or (VIc), R⁷ is —C(O)R¹³, where R¹³ is selected from the group consisting of optionally substituted cycloalkyl (preferably cyclopentyl or cyclohexyl), optionally substituted aryl (preferably phenyl) and optionally substituted heteroaryl (preferably pyrrolyl).

In another set of embodiments of Formula (VIa), (VIb) or (VIc), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In a specific embodiment, a compound of Formula (I) has a structure of Formula (VIIa):

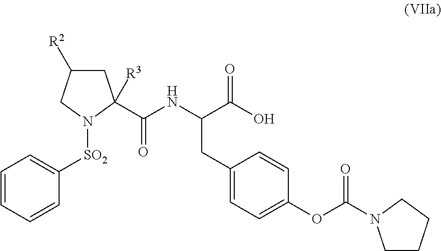

wherein R² and R³ are as defined in Formula (I).

In one set of embodiments of a compound of Formula (I), R³ is H, which provides compounds of the following formula (VIIIa):

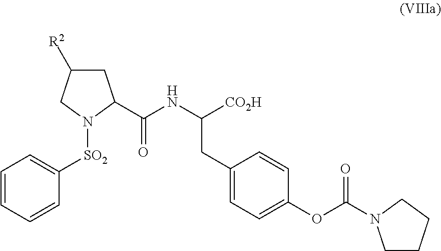

wherein R² is selected from the group consisting of H and a substituent group.

In one form of a compound of Formula (I), R² is H, which provides a compound of the following formula (IXa):

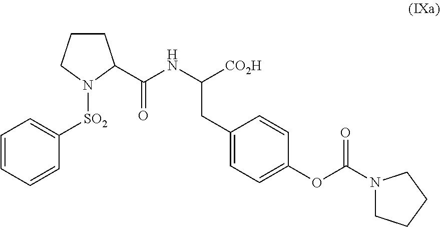 [SMILES: O=C(O)C(Cc1ccc(OC(=O)N2CCCC2)cc1)NC(=O)C1CCCN1S(=O)(=O)c1ccccc1]

or a pharmaceutically acceptable salt thereof.

In a preferred specific embodiment, the compound of Formula (I) is a compound of the following formula (Ic):

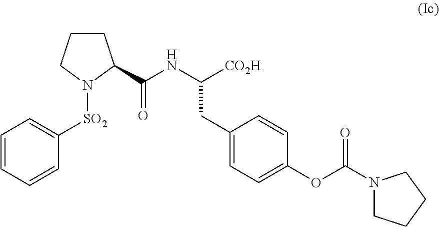 [SMILES: O=C(O)[C@H](Cc1ccc(OC(=O)N2CCCC2)cc1)NC(=O)[C@@H]1CCCN1S(=O)(=O)c1ccccc1]

or a pharmaceutically acceptable salt thereof.

In another specific embodiment, a compound of Formula (I) has a structure of Formula (VIIb):

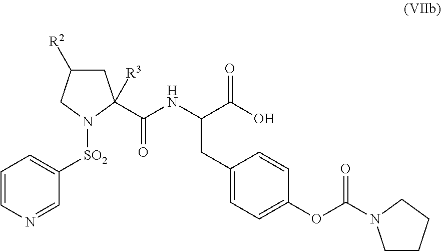

wherein R² and R³ are as defined in Formula (I).

In one set of embodiments of a compound of Formula (I), R³ is H, which provides compounds of the following formula (VIIIb):

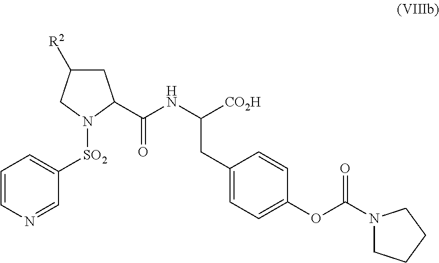

wherein R² is selected from the group consisting of H and a substituent group.

In one form of a compound of Formula (I), R² is H, which provides a compound of the following formula (Id):

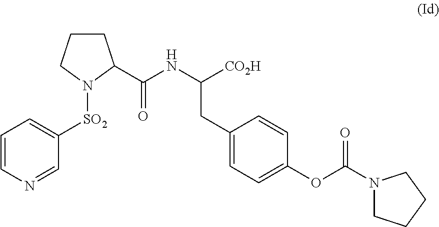 [SMILES: O=C(O)C(Cc1ccc(OC(=O)N2CCCC2)cc1)NC(=O)C1CCCN1S(=O)(=O)c1cccnc1]

or a pharmaceutically acceptable salt thereof.

In a preferred specific embodiment, the compound of Formula (I) is a compound of the following formula (Ie):

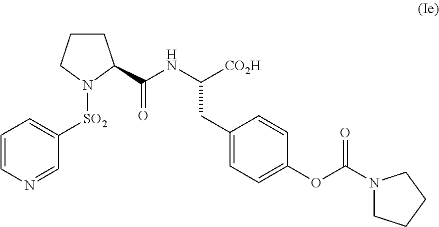 [SMILES: O=C(O)[C@H](Cc1ccc(OC(=O)N2CCCC2)cc1)NC(=O)[C@@H]1CCCN1S(=O)(=O)c1cccnc1]

or a pharmaceutically acceptable salt thereof.

As described herein, in a compound of Formulae (I), (II), (III), (IIIa), (IIIb), (IVa), (IVb), (IVc), (V), (Va), (Vb), (VIa), (VIb), (VIc), (VIIa), (VIIb), (VIIIa) or (VIIIb), R² may in some embodiments be a substituent group.

In one set of embodiments R² is a substituent group selected from the group consisting of optionally substituted heteroaryl, optionally substituted heterocycloalkyl, optionally substituted cycloalkyl, hydroxy, amino and azido, or R² is a substituent having structure of Formula (A):

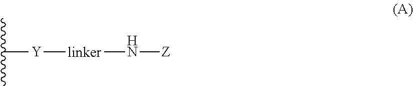

wherein
Y is optionally substituted heteroaryl or optionally substituted heteroaryl-C(O)NH—;
linker is selected from the group consisting of —(CH₂)ₚ— and —(CH₂CH₂O)ₚ—, or any combination thereof;
p at each occurrence is an integer in the range of from 1 to 4; and
Z is a fluorophore (preferably a rhodamine group).

In some embodiments of a compound of Formulae (I), (II), (III), (IIIa), (IIIb), (IVa), (IVb), (IVc), (V), (Va), (Vb), (VIa), (VIb), (VIc), (VIIa), (VIIb), (VIIIa) or (VIIIb), R² is an optionally substituted heteroaryl. Suitable optionally substituted heteroaryl may comprise from 5 to 10 ring atoms and at least one heteroatom selected from the group consisting of O, N, and S. The optionally substituted heteroaryl may be monocyclic or bicyclic.

In some embodiments, R² may be a heteroaryl selected from the group consisting of pyrazole, imidazole, 1,2,3-triazole, 1,2,4-triazole, tetrazole, indazole, 4,5,6,7-tetrahydroindazole and benzimidazole.

In some embodiments of a compound of Formulae (I), (II), (III), (IIIa), (IIIb), (IVa), (IVb), (V), (Va), (Vb), (VIa), (VIb), (VII) or (VIII), R² is an optionally substituted heterocycloalkyl. Suitable optionally substituted heterocycloalkyl may comprise from 3 to 10 ring atoms, preferably from 4 to 8 ring atoms, and at least one heteroatom selected from the group consisting of O, N, and S. The optionally substituted heterocycloalkyl may be monocyclic or bicyclic.

In some embodiments, R² may be an optionally substituted heterocycloalkyl selected from the group consisting of optionally substituted azetidine, pyrrolidine, piperidine, azepane, morpholine and thiomorpholine.

In some embodiments, R² may be optionally substituted piperidine. In some embodiments, the piperidine may be substituted with at least one C₁-C₄ alkyl substituent group. In some embodiments, the C₁-C₄ alkyl substituent group may be methyl.

In some embodiments R² may be selected from the group consisting of 2-methylpiperidine, 3-methylpiperidine, 4-methylpiperidine, 3,5-dimethylpiperidine and 3,3-dimethylpiperidine.

When R² is a optionally substituted heteroaryl or optionally substituted heterocycloalkyl group, R² may be linked to the pyrrolidine ring of the compound of Formulae (I), (II), (III), (IIIa), (IIIb), (IVa), (IVb), (V), (Va), (Vb), (VIa), (VIb), (VII) or (VIII), via a heteroatom on the heteroaryl or heterocycloalkyl ring. For example, when R² is a heteroaryl selected from the group consisting of pyrazole, imidazole, 1,2,3-triazole, 1,2,4-triazole, tetrazole, indazole, 4,5,6,7-tetrahydroindazole and benzimidazole, or when R² is a optionally substituted heterocycloalkyl selected from the group consisting of optionally substituted azetidine, pyrrolidine, piperidine, azepane, morpholine and thiomorpholine, then R² is covalently linked to the remainder of the compound via the nitrogen (N) heteroatom of the heteroaryl or heterocycloalkyl group.

In some embodiments of a compound of Formulae (I), (II), (III), (IIIa), (IIIb), (IVa), (IVb), (V), (Va), (Vb), (VIa), (VIb), (VII) or (VIII), R² is a substituent group having structure of Formula (A):

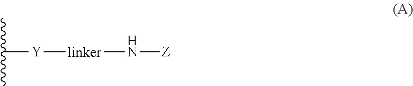

wherein
Y is optionally substituted heteroaryl; or optionally substituted heteroaryl-C(O)NH—;
linker is selected from the group consisting of —(CH₂)ₚ— and —(CH₂CH₂O)ₚ—, or any combination thereof;
p at each occurrence is an integer in the range of from 1 to 4; and
Z is a fluorophore (preferably a rhodamine group).

In some embodiments Y may be selected from the group consisting of triazole or triazole-C(O)NH—.

In some embodiments Y may be triazole or triazole-C(O)NH—, such that the structure of Formula (A) is given by Formula (A1) or (A2):

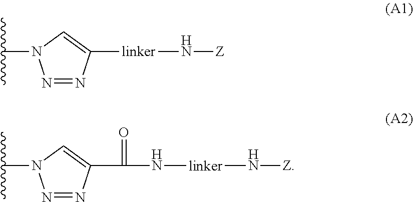

In some embodiments linker may be selected from the group consisting of —(CH₂)ₚ— and —(CH₂CH₂O)ₚ—, or any combination thereof, wherein p at each occurrence is an integer in the range of from 1 to 4.

In some embodiments linker may be given by Formula (A3) or (A4):

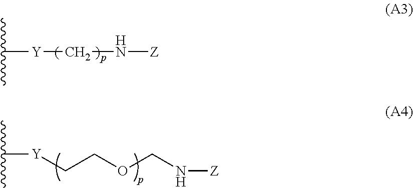

wherein p at each occurrence is an integer in the range of from 1 to 4.

In some embodiments of Formulae (A), (A1), (A2), (A3) or (A4), Z is a rhodamine fluorophore, which is selected from the following group:

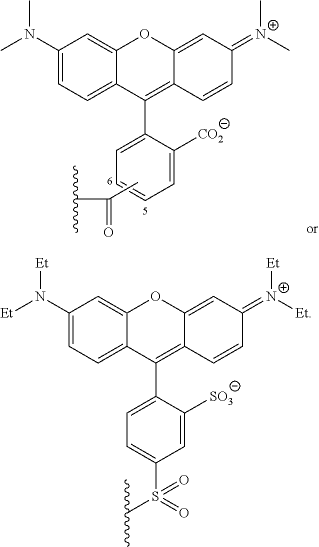

In a specific embodiment, a compound of Formula (I) has the following Formula (IXa):

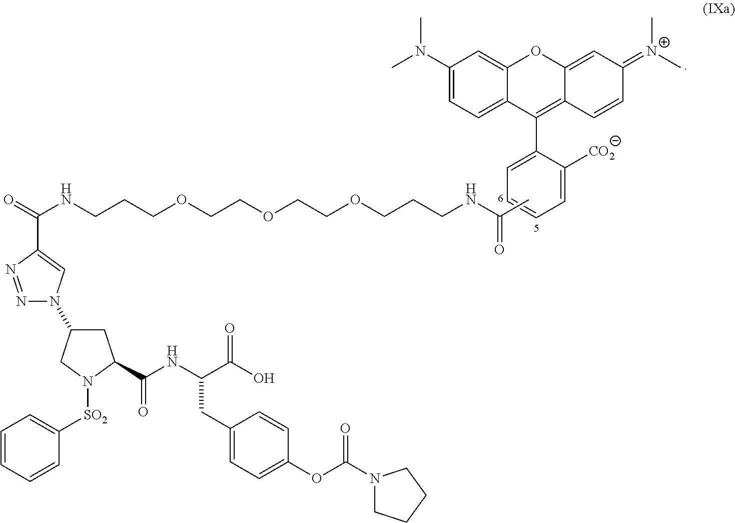

In another specific embodiment, a compound of Formula (I) has the following Formula (IXb):

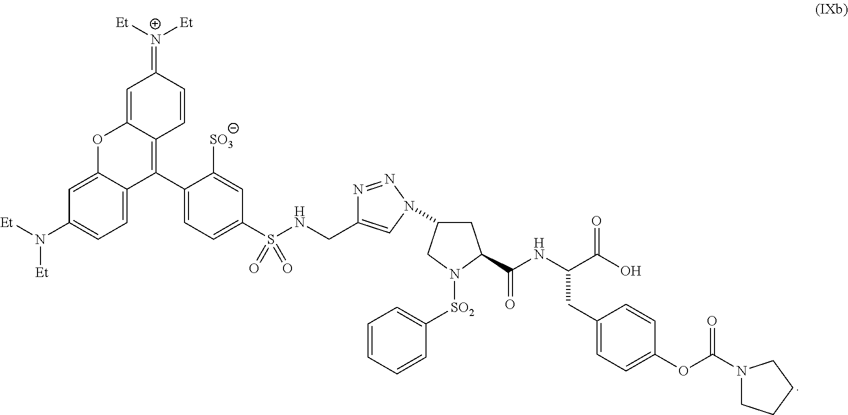

Without wishing to be limited by theory, it is believed that the pyrrolidine carbamate moiety in compounds of formulae described herein is important for ensuring a high binding affinity to an α₉ integrin, more particularly to an α₉β₁ integrin, or an active portion thereof. It is further believed that the carboxylic acid functionality is essential for antagonist activity.

In the above description a number of terms are used which are well known to a skilled addressee. Nevertheless for the purposes of clarity a number of terms are defined as follows.

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, C(═O)OH, —C(═O)R^{e}, C(═O)OR^{e}, C(═O)NR^{e}R^{f}, C(═NOH)R^{e}, C(═NR^{e})NR^{f}R^{g}, NR^{e}R^{f}, NR^{e}C(═O)R^{f}, NR^{e}C(═O)OR^{f}, NR^{e}C(═O)NR^{f}R^{g}, NR^{e}C(═NR^{f})NR^{g}R^{h}, NR^{e}SO₂R^{f}, —SR^{e}, SO₂NR^{e}R^{f}, —OR^{e}, OC(═O)NR^{e}R^{f}, OC(═O)R^{e} and acyl,
wherein R^{e} and R^{f}, R^{g} and R^{h} are each independently selected from the group consisting of H, C₁-C₄alkyl, C₁-C₁₂haloalkyl, C₂-C₁₂alkenyl, C₂-C₁₂alkynyl, C₁-C₁₀ heteroalkyl, C₃-C₆cycloalkyl, C₃-C₁₂cycloalkenyl, C₅-C₆heterocycloalkyl, C₁-C₁₂heterocycloalkenyl, C₆aryl, and C₁-C₅heteroaryl, or R^{e} and R^{f}, when taken together with the atoms to which they are attached form a cyclic or heterocyclic ring system with 3 to 12 ring atoms.

In certain embodiments, optional substituents may be selected from the group consisting of halogen, alkyl, cycloalkyl, heterocycloalkyl, aryl, heteroaryl, arylalkyl, —C(O)R^{e}, —C(O)OR^{e}, —C(O)NR^{e}R^{f}, —OR^{e}, —OR^{e}, —OC(O)NR^{e}R^{f}, OC(O)R^{e} and acyl, wherein R^{e} and R^{f} are each independently selected from the group consisting of H, C₁-C₄alkyl, C₃-C₆cycloalkyl, C₅-C₆heterocycloalkyl, C₆aryl, and C₁-C₅heteroaryl, or R^{e} and R^{f}, when taken together with the atoms to which they are attached form a cyclic or heterocyclic ring system with 3 to 12 ring atoms.

“Alkyl” as a group or part of a group refers to a straight or branched aliphatic hydrocarbon group, preferably a C₁-C₁₂ alkyl, more preferably a C₁-C₁₀ alkyl, most preferably C₁-C₄ unless otherwise noted. Examples of suitable straight and branched C₁-C₄ alkyl substituents include methyl, ethyl, n-propyl, 2-propyl, n-butyl, sec-butyl and t-butyl. The group may be a terminal group or a bridging group.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (II) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

A “bond” is a linkage between atoms in a compound or molecule. In one set of embodiments of a compound of Formula (I) as described herein, the bond is a single bond.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems (such as cyclohexyl), bicyclic systems such as decalin, and polycyclic systems such as adamantane. A cycloalkyl group typically is a C₃-C₁₂ alkyl group. The group may be a terminal group or a bridging group.

“Halogen” represents chlorine, fluorine, bromine or iodine.

“Heteroaryl” either alone or part of a group refers to groups containing an aromatic ring (preferably a 5- or 6-membered aromatic ring) having one or more heteroatoms as ring atoms in the aromatic ring with the remainder of the ring atoms being carbon atoms. Suitable heteroatoms may be selected from the group consisting of nitrogen, oxygen and sulphur. The group may be a monocyclic or bicyclic heteroaryl group. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl, 1-, 2-, or 3-indolyl, and 2- or 3-thienyl. A heteroaryl group is typically a C₁-C₁₈ heteroaryl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4- to 7-membered. Examples of suitable heterocycloalkyl include pyrrolidinyl, tetrahydrofuryl, tetrahydrothiofuranyl, piperidyl, piperazyl, tetrahydropyranyl and morpholino. The group may be a terminal group or a bridging group.

It is understood that included in the family of compounds of Formula (I) are isomeric forms including diastereomers, enantiomers and tautomers, and geometrical isomers in “E” or “Z” configuration or a mixture of E and Z isomers. It is also understood that some isomeric forms such as diastereomers, enantiomers, and geometrical isomers can be separated by physical and/or chemical methods and by those skilled in the art. For those compounds where there is the possibility of geometric isomerism the applicant has drawn the isomer that the compound is thought to be although it will be appreciated that the other isomer may be the correct structural assignment.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

Formula (I) is further intended to encompass pharmaceutically acceptable salts of the compounds.

The term “pharmaceutically acceptable salt” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic, carboxylic, and sulfonic classes of organic acids, examples of which are formic, acetic, propanoic, succinic, glycolic, gluconic, lactic, malic, tartaric, citric, fumaric, maleic, alkyl sulfonic, and arylsulfonic. In a similar vein base addition salts may be prepared by ways well known in the art using organic or inorganic bases. Examples of suitable organic bases include simple amines such as methylamine, ethylamine, triethylamine and the like. Examples of suitable inorganic bases include NaOH, KOH, and the like. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

In another preferred embodiment of the invention, there is provided a method for enhancing release of HSC and their precursors and progenitors thereof from a BM stem cell binding ligand in vivo or ex vivo, said method comprising administering in vivo or ex vivo an effective amount of an antagonist of an α₉ integrin or an active portion thereof and a CXCR4 antagonist or an active portion thereof to the BM stem cell niche.

Once the HSC dislodge from the BM stem cell binding ligand they are no longer anchored to the BM and available to be released from the BM and enter a cell cycle toward proliferation and differentiation. Alternatively, they can remain in the BM and enter a cell cycle in the BM.

In a further preferred embodiment, the present invention there is provided a method for enhancing mobilization of HSC and their precursors and progenitors thereof from a BM stem cell niche in vivo or ex vivo, said method comprising administering in vivo or ex vivo an effective amount of an antagonist of α₉ integrin or an active portion thereof and a CXCR4 antagonist or an active portion thereof to the BM stem cell niche.

By virtue of the HSC becoming dislodged and released, the HSC become available to be mobilized to the PB. The dislodgement and release is essential to enable mobilization. An enhanced release of the HSC will enable more cells as a consequence to be mobilized.

In another preferred embodiment of the invention, the methods are conducted in the presence or absence of G-CSF. Preferably, the methods are conducted in the absence of G-CSF.

Although clinically G-CSF is the most extensively used mobilization agent for HSC, its drawbacks include potentially toxic side effects, a relatively long course of treatment (5-7 days of consecutive injections), and variable responsiveness of patients. Therefore, an advantage of the invention is that effective mobilization can occur in the absence of G-CSF which substantially can avoid the toxic side effects.

The CXC chemokine receptor 4 (CXCR4), which is a 7 transmembrane protein, coupled to guanine nucleotide-binding protein. CXCR4 is widely expressed on cells of haematopoietic origin, and is a major co-receptor with CD4+ for human immunodeficiency virus 1 (HIV-1). Under normal physiological conditions, CXCR4 is mainly expressed in the hematopoietic and immune systems.

CXCR4 is specific for chemokine ligand 12 (CXCL12), which is also called stromal-derived-factor-1 (SDF-1). As a homeostatic chemokine, SDF-1 is an 8 kDa chemokine peptide. Like other chemokines, SDF-1 binds to its receptors to promote directional migration of cells to specific locations (chemotaxis) with 67 amino acid residues, mainly localized in bone marrow stromal cells.

CXCR4 antagonists have been developed to block SDF-1/CXCR4 interactions. The CXCR4 antagonist, Plerixafor, was approved by the FDA in 2008 for the mobilization of hematopoietic stem cells.

Suitable CXCR4 antagonists to be used with BOP include, but are not limited to, bicyclam derivatives (e.g. AMD3100), tetrahydroquinoline derivatives (e.g. AMD070, AMD11070 and GSK812397), cyclic peptides (e.g. T140, TC14012, TN14003, FC131 and FC122), para-xylyl-enediamine-based derivatives (e.g. AMD36465, WZ811 and MSXI22), isothiourea derivatives and other CXCR4 antagonists such as POL6326, POL5551, CCTE-9908 and TG-0054. Preferably, the CR+XCR4 antagonist is AMD3100.

AMD3100 (I,r-[I,4-Phenylenebis(methylene)]bis [1,4,8,11-tetraazacyclotetradecane]octohydrobromide dehydrate; also known as Plerixafor) is a known CXCR4 antagonist that has been approved for the mobilization of haematopoietic stem cells by the U.S. Food and Drug Administration. While it has been established that AMD3100 is an antagonist of CXCR4 in vitro, it also appears to have more activities than simple CXCR4 antagonism in vivo.

“Haematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. Hence the terms “haematopoietic stem cells”, “HSC”, “haematopoietic progenitors”, “HPC”, “progenitor cells” or “blast cells” are used interchangeably in the present invention and describe HSCs with reduced differentiation potential, but are still capable of maturing into different cells of a specific lineage, such as myeloid or lymphoid lineage. “Haematopoietic progenitors” include erythroid burst forming units, granulocyte, erythroid, macrophage, megakaryocyte colony forming units, granulocyte, erythroid, macrophage, and granulocyte macrophage colony-forming units.

The present invention relates to enhancing the dislodgment of HSC and their precursors and progenitors thereof from a BM stem cell binding ligand. Once dislodged, the cells can be released from the BM stem cell niche where they can remain or preferably be released and mobilized to the PB. These cells have haematopoietic reconstitution capacity. The present invention provides a method to enhance mobilization of HSC assisted by the dislodgement of the HSC from the BM stem cell niche preferably nearest the bone/BM interface within the endosteal niche or from the central medullary cavity. More preferably, the HSC are mobilized from the bone/BM interface within the endosteal niche as it is these cells that have been shown to give greater long term, multi-lineage haematopoietic reconstitution relative to HSC isolated from the central medullary cavity.

The type of cells that are dislodged, released or mobilized may also be found in murine populations selected from the group including BM derived progenitor enriched Lin-Sca-1+ckit+(herein referred to as LSK) cells or stem cell enriched LSKCD150+CD48− cells (herein referred to as LSKSLAM). These equivalent murine populations provide an indication of the cell types that can be dislodged, released or mobilized from the BM stem cell niche by the use of an antagonist of an α₉ integrin or an active portion thereof. Preferably, the cell types are equivalent to those found in a stem cell enriched LSKCD150+CD48− cells (LSKSLAM).

Preferably, the cells that are dislodged, released or mobilized are endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells. Most preferably they are human cells.

The present invention may be conducted in vivo or ex vivo. That is the antagonist of α₉, preferably an antagonist of α₉β₁, more preferably an antagonist of α₉β₁/α₄β₁ can be administered with a CXCR4 antagonist or an active portion thereof to a subject in need in vivo or to an ex vivo sample to mobilize HSC from the BM.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

The present invention relates to enhancing HSC dislodgement, release or mobilization. “Enhancement,” “enhance” or “enhancing” as used herein refers to an improvement in the performance of or other physiologically beneficial increase in a particular parameter of a cell or organism. At times, enhancement of a phenomenon may be quantified as a decrease in the measurements of a specific parameter. For example, migration of stem cells may be measured as a reduction in the number of stem cells circulating in the circulatory system, but this nonetheless may represent an enhancement in the migration of these cells to areas of the body where they may perform or facilitate a beneficial physiologic result, including, but not limited to, differentiating into cells that replace or correct lost or damaged function. At the same time, enhancement may be measured as an increase of any one cell type in the peripheral blood as a result of migration of the HSC from the BM to the PB. Enhancement may refer to a 15%, 20%, 25%, 30%, 35%, 40%, 45% or greater than 50% reduction in the number of circulating stem cells or in the alternative may represent a 15%, 20%, 25%, 30%, 35%, 40%, 45% or greater than 50% increase in the number of circulating stem cells. Enhancement of stem cell migration may result in or be measured by a decrease in a population of the cells of a non-haematopoietic lineage, such as a 15%, 20%, 25%, 30%, 35%, 40%, 45%, 50%, 55%, 60%, 70%, 75% or greater decrease in the population of cells or the response of the population of cells. Put another way, an enhanced parameter may be considered as the trafficking of stem cells. In one embodiment, the enhanced parameter is the release of stem cells from a tissue of origin such as the BM. In one embodiment, an enhanced parameter is the migration of stem cells. In another embodiment, the parameter is the differentiation of stem cells.

In one embodiment, the α₉ integrin antagonist and a CXCR4 antagonist or an active portion thereof is administered intravenously, intradermally, subcutaneously, intramuscularly, transdermally, or transmucosally; optionally the antagonist is administered intravenously or subcutaneously.

In another embodiment the α₉ integrin antagonist and the CXCR4 antagonist are administered separately or in combination such that the administration of the α₉ integrin antagonist and the CXCR4 antagonist can act together to provide a synergistic result for the mobilization of the HSC from the BM to the PB. The α₉ integrin antagonist and the CXCR4 antagonist may be administered in combination, simultaneously or sequentially.

In yet another aspect of the invention there is provided a composition for use in enhancing dislodgement of HSC from a BM stem cell binding ligand in a BM stem cell niche said composition comprising an antagonist of α₉ integrin as herein described and a CXCR4 antagonist or an active portion thereof. More preferably, the antagonist is an α₉ integrin antagonist as herein described. Most preferably the antagonist is an α₄β₁/α₉β₁ integrin antagonist as herein described.

Preferably the CXCR4 antagonist is AMD 3100 or an active portion thereof.

In a preferred embodiment, the composition enhances release of HSC from a BM stem cell binding ligand in a BM stem cell niche. More preferably, the composition enhances mobility or mobilization of HSC from a BM stem cell niche to the PB.

The composition may be a pharmaceutical composition further including a pharmaceutically acceptable carrier. The antagonists of as integrin as described herein may be provided in the composition separately or in combination with a further antagonist of α₉ integrin, α₄ integrin, α₉β₁ integrin, α₄β₁ integrin or it may be a combined antagonist of α₉β₁/α₄β₁ integrin. The antagonists may be the same or different, but will all act as antagonists of at least the α₉ integrin.

In another aspect of the present invention there is provided a use of an antagonist of as integrin as described herein and a CXCR4 antagonist or an active portion thereof in the preparation of a medicament for enhancing dislodgement of HSC and their precursors and progenitors thereof from a BM stem cell binding ligand in a patient.

The methods described herein include the manufacture and use of compositions and pharmaceutical compositions, which include antagonists of α₉ integrin as described herein and a CXCR4 antagonist or an active portion thereof as active ingredients for enhancing dislodgement of HSC and their precursors and progenitors thereof from a BM stem cell binding ligand. Preferably the release of the HSC is enhanced. More preferably, the HSC mobilization is enhanced. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration that are known to the skilled addressee. Supplementary active compounds can also be incorporated into the compositions, e.g., growth factors such as G-CSF. More specific carriers including cyclodextrin, preferably propylcyclodextrin, more preferably hydroxylpropylcyclodextrin may be used. This may be present in a range of 0-20%, preferably 5, 6, 7, 8, 9, 10, 11, 12, 13, 14 or 15%, more preferably 10%.

Pharmaceutical compositions are typically formulated to be compatible with the intended route of administration. Examples of routes of administration include parenteral, e.g., intravenous, intradermal, subcutaneous, transdermal (topical), transmucosal, intraperitoneal and rectal administration. Preferably, the antagonists of α₉ integrin as described herein and a CXCR4 antagonist or an active portion thereof are administered subcutaneously or intravenously. These compounds may be administered in combination, simultaneously or sequentially.

In some embodiments, the pharmaceutical compositions are formulated to target delivery of the antagonists of α₉ integrin as described herein and a CXCR4 antagonist or an active portion thereof to the bone marrow preferably to the BM stem cell niche, and more preferably to the endosteal niche of the BM stem cell niche. For example, in some embodiments, the antagonists of α₉ integrin as described herein in combination with a CXCR4 antagonist or an active portion thereof may be formulated in liposomes nanosuspensions and inclusion complexes (e.g. with cyclodextrins), which can effect more targeted delivery to the BM while reducing side effects.

The pharmaceutical compositions can be included in a container, pack, or dispenser together with instructions for administration.

In yet another aspect of the invention there is provided a method of harvesting HSC from a subject said method comprising:
administering an effective amount of an antagonist of α₉ integrin or an active portion thereof as described herein and a CXCR4 antagonist or an active portion thereof to a subject wherein said effective amount enhances dislodgement of HSC and their precursors and progenitors thereof from a BM stem cell binding ligand in a BM stem cell niche;
mobilizing the dislodged HSC to PB; and
harvesting the HSC from the PB.

Preferably, the α₉ integrin antagonist and a CXCR4 antagonist or an active portion thereof is administered in the absence of G-CSF.

The use of compounds such as α₉β₁ integrin antagonist as herein described and a CXCR4 antagonist or an active portion thereof to enhance dislodgement of HSC and their precursors and progenitors thereof from a BM stem cell binding ligand in the BM stem cell niche allows for the cells to eventually mobilize to the PB for further collection. The cells may naturally mobilize and egress from the BM or they may be stimulated to mobilize by the use of other HSC mobilizing agents such as, but not limited to interleukin-17, cyclophosphamide (Cy), Docetaxel and granulocyte-colony stimulating factor (G-CSF).

In one embodiment, it is considered that the cells once harvested can be returned to the body to supplement or replenish a patient's haematopoietic progenitor cell population (homologous or autologous transplantation) or alternatively be transplanted to another patient to replenish their haematopoietic progenitor cell population (heterologous or allogeneic transplantation). This can be advantageous, in the instance following a period where an individual has undergone chemotherapy. Furthermore, there are certain genetic conditions such as thalassemias, sickle cell anemia, Dyskeratosis congenital, Shwachman-Diamond syndrome, and Diamond-Blackfan anemia wherein HSC and HPC numbers are decreased. Hence the methods of the invention in enhancing HSC dislodgement, release or mobilization may be useful and applicable.

The recipient of a bone marrow transplant may have limited bone marrow reserve such as an elderly subject or a subject previously exposed to an immune depleting treatment such as chemotherapy. The subject may have a decreased blood cell level or is at risk for developing a decreased blood cell level as compared to a control blood cell level. As used herein the term control blood cell level refers to an average level of blood cells in a subject prior to or in the substantial absence of an event that changes blood cell levels in the subject. An event that changes blood cell levels in a subject includes, for example, anaemia, trauma, chemotherapy, bone marrow transplant and radiation therapy. For example, the subject has anaemia or blood loss due to, for example, trauma.

Typically, an effective amount of an α₉ integrin antagonist such as an α₉β₁ integrin antagonist, more preferably an α₉β₁/α₄β₁ integrin antagonist and a CXCR4 antagonist is administered to a donor to induce dislodgement, release or preferably mobilization of HSC from the BM and release and mobilize to the PB. Once the HSC are mobilized to the PB, collection of the blood and separation of HSC can proceed using methods generally available for blood donation as used, such as, but not limited to those techniques employed in Blood Banks. In some embodiments, once PB or BM is obtained from a subject who has been treated using an antagonist of α₉ integrin as described herein, the HSC can be isolated therefrom using a standard method such as apheresis or leukapheresis.

Preferably the effective amount of the integrin antagonist for humans is in the range of 25-1000 μg/kg body weight, more preferably 50-500 μg/kg body weight, most preferably 50-250 μg/kg body weight. The effective amount may be selected from the group including 50, 60, 70, 80, 90, 100, 110, 120, 130, 140, 150, 160, 170, 180, 190, 200, 210, 220, 230, 240 or 250 μg/kg body weight.

Preferably the effective amount of the CXCR4 antagonist for humans is in the range of 10-1000 ug/kg body weight, more preferably 10-500 ug/kg body weight, most preferably 10-250 ug/kg body weight. The effective amount may be selected from the group including 10, 20, 30, 40, 50, 60, 70, 80, 90, 100, 110, 120, 130, 140, 150, 160, 170, 180, 190, 200, 210, 220, 230, 240 or 250 μg/kg body weight.

Dislodgement, release or preferably mobilization may occur immediately, depending on the amount of α₉ integrin antagonist and a CXCR4 antagonist or an active portion thereof used. However, the HSC may be harvested in approximately 1 hours' time after administration. The actual time and amount of the α₉ integrin antagonist and a CXCR4 antagonist or an active portion thereof may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine an effective amount through routine experimentation and use of control curves.

As considered in the present invention, the term “control curve” is considered to refer to statistical and mathematically relevant curves generated through the measurement of HSC dislodgement, release or mobilization characteristics of different concentrations of α₉ integrin antagonist and a CXCR4 antagonist or an active portion thereof under identical conditions, and wherein the cells can be harvested and counted over regular time intervals. These “control curves” as considered in the present invention can be used as one method to estimate different concentrations for administering in subsequent occasions.

As considered in the present invention, the terms “harvesting haematopoietic stem cells”, “harvesting haematopoietic progenitor cells”, “harvesting HSC” or “harvesting HPC” are considered to refer to the separation of cells from the PB and are considered as techniques to which the person skilled in the art would be aware. The cells are optionally collected, separated, and optionally further expanded generating even larger populations of HSC and differentiated progeny.

In another aspect of the present invention, there is provided a cell composition comprising HSC obtained from a method as described herein said method comprising administering an effective amount of an antagonist of ca integrin as herein described and a CXCR4 antagonist or an active portion thereof to enhance dislodgement, release or mobilization of HSC from the BM to the PB.

As a consequence of enhanced dislodgement of the HSC, it is postulated that more HSC can be released to the BM stem cell niche for subsequent mobilization to the PB. Therefore the cell compositions harvested from a subject that has been administered an effective amount of an antagonist of an α₉ integrin or an active portion thereof to the BM stem cell niche will be enriched with HSC.

Preferably the cell composition will be enriched with cells of the endosteal niche and are endosteal progenitor cells selected from the group comprising CD34⁺, CD38⁺, CD90⁺, CD133⁺, CD34⁺CD38⁻ cells, lineage-committed CD34⁻ cells, or CD34⁺CD38⁺ cells.

In yet another aspect of the present invention there is provided a method for the treatment of haematological disorders said method comprising administering a cell composition comprising HSC obtained from a method as described herein said method comprising administering an effective amount of an antagonist of α₉ integrin as described herein and a CXCR4 antagonist or an active portion thereof to enhance dislodgement, release or mobilization of HSC from the BM to the PB.

In yet another aspect of the present invention there is provided a method for the treatment of haematological disorders in a subject said method comprising administering a therapeutically effective amount of an antagonist of α₉ integrin as described herein and a CXCR4 antagonist or an active portion thereof to the subject to enhance dislodgement, release or mobilization of HSC from the BM to the PB.

In yet another preferred embodiment, the haematological disorder is a haematopoietic neoplastic disorder and the method involves chemosensitizing the HSC to alter susceptibility of the HSC, such that a chemotherapeutic agent, having become ineffective, becomes more effective.

A long standing issue in the treatment of leukemia is the concept that malignant cells in a dormant state are likely to evade the effects of cytotoxic agents, rendering them capable of driving relapse. Whilst much effort has gone into understanding the control of cancer cell dormancy, very little has concentrated on the role of the microenvironment and in particular the bone marrow stem cell niche. Recently, data has emerged demonstrating that the extracellular matrix molecule Osp, known to anchor normal haematopoietic stem cells in the bone marrow, also plays a role in supporting leukaemic cells, in particular acute lymphoblastic leukaemia (ALL), dormancy by anchoring these in key regions of the bone marrow microenvironment. Furthermore, additional data shows that relapsed ALL have significantly elevated levels of the integrin α₄β₁. These data provided herein suggest that an agent that competes with the interaction of α₉β₁ and its extracellular matrix ligands will induce these cells into cell cycle, rendering them vulnerable to cytotoxic chemotherapy. Accordingly, BOP or a α₉ antagonist, preferably an α₉β₁ antagonist, more preferably an α₉β₁/α₄β₁ antagonist can be used in combination with any CXCR4 antagonist to either enhance the dislodgement, release and mobilisation of HSC into the peripheral blood or increase the sensitivity of leukemic cells to chemotherapy.

The methods described herein include in some embodiments methods for the treatment of subjects with haematological disorders who are in need of increased numbers of stem cells. In some further embodiments, the subject is scheduled to or intends to donate stem cells such as HSC e.g., for use in heterologous or autologous transplantation. Generally, the methods include administering a therapeutically effective amount of an antagonist of α₉ integrin as described herein and a CXCR4 antagonist or an active portion thereof, to a subject who is in need of, or who has been determined to be in need of, such treatment. Administration of a therapeutically effective amount of antagonists of α₉ integrin as described herein and a CXCR4 antagonist or an active portion thereof for the treatment of such subjects will result in an increased number and/or frequency of HSC in the PB or BM.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

An “effective amount” is an amount sufficient to effect a significant increase or decrease in the number and/or frequency of HSC in the PB or BM. An effective amount can be administered in one or more administrations, applications or dosages.

“Therapeutically effective amount” as used herein refers to the quantity of a specified composition, or active agent in the composition, sufficient to achieve a desired effect in a subject being treated. For example, this can be the amount effective for enhancing migration of HSC that replenish, repair, or rejuvenate tissue. In another embodiment, a “therapeutically effective amount” is an amount effective for enhancing trafficking of HSC, such as increasing release of HSC, as can be demonstrated by elevated levels of circulating stem cells in the bloodstream. In still another embodiment, the “therapeutically effective amount” is an amount effective for enhancing homing and migration of HSC from the circulatory system to various tissues or organs, as can be demonstrated be a decreased level of circulating HSC in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

The compositions can be administered one from one or more times per day to one or more times per week; including once every other day. The skilled artisan will appreciate that certain factors may influence the dosage and timing required to effectively treat a subject, including but not limited to previous treatments, the general health and/or age of the subject, and other diseases present. Moreover, treatment of a subject with an effective amount of the compositions described herein can include a single treatment or a series of treatments.

In some embodiments, such administration will result in an increase of about 10-200-fold in the number of HSC in the PB.

In some embodiments, such administration will result in an increase of about 2-6 fold increase in CD34+ cells in the PB.

Dosage, toxicity and therapeutic efficacy of the compounds can be determined by standard pharmaceutical procedures, e.g., in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the antagonists of α₉ integrin as described herein that achieves a half-maximal inhibition of symptoms) as determined in cell culture. Such information can be used to more accurately determine useful doses in humans. Levels in plasma may be measured, for example, by high performance liquid chromatography.

In some embodiments, the methods of treatment described herein include administering another HSC mobilizing agent, e.g., an agent selected from the group consisting of, but not limited to, interleukin-17, cyclophosphamide (Cy), Docetaxel and granulocyte-colony stimulating factor (G-CSF). In some embodiments, once PB or BM is obtained from a subject who has been treated using an antagonist of α₉ integrin as described herein, the HSC can be isolated therefrom, e.g., using a standard method such as apheresis or leukapheresis.

In some embodiments, the methods include administering the isolated stem cells to a subject, such as reintroducing the cells into the same subject or transplanting the cells into a second subject, e.g., an HLA type-matched second subject, an allograft.

The present invention includes administering an α₉ integrin antagonist directly to a patient to mobilize their own HSC or using HSC from another donor treated with an α₉ integrin antagonist from which HSC have been harvested.

In some embodiments, the subject administered an antagonist of α₉ integrin as described herein and a CXCR4 antagonist or an active portion thereof is healthy. In other embodiments, the subject is suffering from a disease or physiological condition, such as immunosuppression, chronic illness, traumatic injury, degenerative disease, infection, or combinations thereof. In certain embodiments, the subject may suffer from a disease or condition of the skin, digestive system, nervous system, lymph system, cardiovascular system, endocrine system, or combinations thereof.

In specific embodiments, the subject suffers from osteoporosis, Alzheimer's disease, cardiac infarction, Parkinson's disease, traumatic brain injury, multiple sclerosis, cirrhosis of the liver, or combinations thereof.

Administration of a therapeutically effective amount of an antagonist of α₉ integrin as described herein and a CXCR4 antagonist or an active portion thereof may prevent, treat and/or lessen the severity of or otherwise provide a beneficial clinical benefit with respect to any of the aforementioned conditions, although the application of the methods and use of the an antagonist of α₉ integrin as described herein is not limited to these uses. In various embodiments, the novel compositions and methods find therapeutic utility in the treatment of, among other things, skeletal tissues such as bone, cartilage, tendon and ligament, as well as degenerative diseases, such as Parkinson's and diabetes. Enhancing the release, circulation, homing and/or migration of stem cells from the blood to the tissues may lead to more efficient delivery of HSC to a defective site for increased repair efficiency.

In some embodiments subjects that can usefully be treated using the HSC, PB or BM include any subjects who can be normally treated with a bone marrow or stem cell transplant, e.g., subjects who have cancers, e.g., neuroblastoma (cancer that arises in immature nerve cells and affects mostly infants and children), myelodysplasia, myelofibrosis, breast cancer, renal cell carcinoma, or multiple myeloma. For example, the cells can be transplanted into subjects who have cancers that are resistant to treatment with radiation therapy or chemotherapy, e.g., to restore stem cells that were destroyed by high doses of chemotherapy and/or radiation therapy used to treat the cancers or non-responders to G-CSF treatment to mobilize HSC.

In some embodiments, the subject has a haematopoietic neoplastic disorder. As used herein, the term “haematopoietic neoplastic disorders” includes diseases involving hyperplastic/neoplastic cells of haematopoietic origin, e.g., arising from myeloid, lymphoid or erythroid lineages, or precursor cells thereof. In some embodiments, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not Limited to Hodgkin's Disease and Medium/High grade (aggressive) Non-Hodgkin's lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. In general, the methods will include administering the cell compositions, or dislodging, releasing or mobilizing stem cells to restore stem cells that were destroyed by high doses of chemotherapy and/or radiation therapy, e.g., therapy used to treat the disorders. Alternatively, the HSC are dislodged, released or mobilized from the BM stem cell niche and chemosensitized whilst entering a cell cycle either in the BM or the PB. Preferably, the haematopoietic neoplastic disorder is ALL.

In some embodiments, the BM, PB or HSC are used to treat a subject who has an autoimmune disease, e.g., multiple sclerosis (MS), myasthenia gravis, autoimmune neuropathy, scleroderma, aplastic anemia, and systemic lupus erythematosus.

In some embodiments, the subject who is treated has a non-malignant disorder such as aplastic anemia, a hemoglobinopathy, including sickle cell anemia, or an immune deficiency disorder.

The present invention further provides a dosing regimen. In one embodiment, the dosing regimen is dependent on the severity and responsiveness of a disease state to be treated, with the course of treatment lasting from a single administration to repeated administration over several days and/or weeks. In another embodiment, the dosing regimen is dependent on the number of circulating CD34+ HSCs in the peripheral blood stream of a subject. In another embodiment, the dosing regimen is dependent on the number of circulating bone marrow-derived stem cells in the peripheral blood stream of a subject. For instance, the degree of mobility of the HSC from the BM may be dependent on the number of HSC already circulating in the PB.

The present invention further provides a method of enhancing the trafficking of HSC in a subject said method comprising administering a therapeutically effective amount of an antagonist of ca integrin as herein described and a CXCR4 antagonist or an active portion thereof to a subject. In one embodiment, the level of trafficking of HSC relates to the number of circulating CD34⁺ HSCs in the peripheral blood of a subject. In another embodiment, the level of trafficking of HSC relates to the number of circulating bone marrow-derived HSCs in the peripheral blood of a subject.

The present invention further provides a method of inducing a transient increase in the population of circulating HSC, such as endosteal progenitor cells and are selected from the group comprising CD34⁺, CD38⁴, CD90⁺, CD133⁺, CD34⁺CD38⁻ cells, lineage-committed CD34⁻ cells, or CD34⁺CD38⁺ cells following administration of an antagonist of as integrin as described herein and a CXCR4 antagonist or an active portion thereof to a subject. In one embodiment, providing an antagonist of ca integrin as described herein and a CXCR4 antagonist or an active portion thereof to a subject will enhance release of that subject's HSC within a certain time period, such as less than 12 days, less than 6 days, less than 3 days, less than 2 days, or less than 1 day, less than 12 hours, less than 6 hours, less than about 4 hours, less than about 2 hours, or less than about 1 hour following administration.

In one embodiment, administration of an antagonist of α₉ integrin as described herein and a CXCR4 antagonist or an active portion thereof results in the HSC into the circulation from about 30 minutes to about 90 minutes following administration. Preferably, the release of HSC will be about 60 minutes following administration. In another embodiment, released HSC enter the circulatory system and increase the number of circulating HSC within the subject's body. In another embodiment, the percentage increase in the number of circulating HSC compared to a normal baseline may be about 25%, 30%, 40%, 50%, 60%, 70%, 80%, 90% or about 100% or greater than about 100% increase as compared to a control. In one embodiment, the control is a base line value from the same subject. In another embodiment, the control is the number of circulating stem cells or HSC in an untreated subject, or in a subject treated with a placebo or a pharmacological carrier.

In another aspect of the invention there is provided a method of transplanting HSC into a patient, said method comprising
administering an α₉ integrin antagonist and a CXCR4 antagonist to a subject to dislodge HSC from a BM stem cell binding ligand;
releasing and mobilizing the HSC from the BM to the PB;
harvesting HSC from the PB from the subject; and
transplanting the HSC to the patient.

In one embodiment, it is considered that the cells once harvested provide a cell composition that can be returned to the body to supplement or replenish a subject's haematopoietic progenitor cell population or alternatively be transplanted to another subject to replenish their haematopoietic progenitor cell population. This can be advantageous, in the instance following a period where an individual has undergone chemotherapy.

In one embodiment the method relates specifically to transplanting a subset of HSC. These cells have haematopoietic reconstitution capacity and reside in BM in the stem cell niche. The present invention provides a method to transplant the HSC from the stem cell niche preferably nearest the bone/BM interface within the endosteal niche or from the central medullary cavity. More preferably, the HSC are transplanted from the bone/BM interface within the endosteal niche as it is these cells that have been shown to give greater long term, multi-lineage haematopoietic reconstitution relative to HSC isolated from the central medullary cavity. Preferably the cells that are transplanted are found in the stem cell niche, more preferably the central or endosteal niche.

The equivalent type of cells that may be transplanted may also be found in murine populations selected from the group including BM derived progenitor enriched Lin-Sca-1+ckit+(herein referred to as LSK) cells or stem cell enriched LSKCD150+CD48-cells (herein referred to as LSKSLAM).

Preferably, the cells that are transplanted are endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells.

In summary, applicants demonstrate that BOP, a small molecule inhibitor of α₄β₁ and α₉β₁ integrins, effectively and rapidly mobilized HSC with long-term multi-lineage engraftment potential. When used in combination with CXCR4 inhibitors such as AMD3100, significant enhancement in the mobilization of long-term repopulating HSC is observed relative to G-CSF. The efficacy of HSC mobilization using the BOP/AMD3100 combination was corroborated in the mobilization of CD34⁺ cells in a humanised NODSCIDIL2Rγ^{-/-} model. Using the related fluorescent labelled integrin antagonist R-BC154 (IXb), applicants show that this class of compounds bind murine and human HSC and progenitors via activated α₉β₁ and α₉β₁ integrins within the endosteal niche. Thus, therapeutic targeting of the endosteal niche using small molecule α₄β₁ and α₉β₁ integrin antagonists either alone or in combination with AMD3100 offers an effective and convenient strategy that addresses many of the shortcomings associated with G-CSF in clinical HSC mobilization.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

The present invention will now be more fully described by reference to the following non-limiting Examples.

### EXAMPLES

### Methods

(i) Flow Cytometry

Flow cytometric analysis was performed using an LSR II (BD Biosciences) as previously described in J. Grassinger, et al Blood, 2009, 114, 49-59. R-BC154 (IXb) was detected at 582 nm, 585 nm, or 610 nm and excited with the yellow-green laser (561 nm). For BM and PB analysis, up to 5×10⁶ cells were analysed at a rate of 10-20 k cell events/sec. For analysis of PB LSKSLAM, up to 1×10⁶ events were saved. Cell sorting was performed on a Cytopeia Influx (BD) as previously described in J. Grassinger, et al.

(ii) Cell Lines

Stable LN18 cells ((ATCC number: CRL-2610) over-expressing integrin α₄β₁ (LN18 α₄β₁) or α₉β₁ (LN18 α₉β₁)) were generated by retroviral transduction using the pMSCV-hITGA4-IRES-hITGB1 and pMSCV-hITGA9-IRES-hITGB1 vectors as previously described in J Grassinger, et al Blood, 2009, 114, 49-59 and were maintained in DMEM supplemented with 2 mM L-glutamate in 10% FBS. Transduced cells were selected by two rounds of FACS using 2.5 μg ml-1 PE-Cy5-conjugated mouse-anti-human α₄ antibody (BD Bioscience) or 20 μg ml-1 of mouse-anti-human α₉β₁ antibody (Millipore) in PBS-2% FBS, followed by 0.5 μg ml-1 of PE-conjugated goat-anti-mouse IgG (BD Bio-science). Silencing of α₄ expression in LN18 and LN18 α₉β₁ cells was performed as described above using pSM2c-shITGA4 (Open Biosystems). α₄-silenced LN18 cells (control cell line; LN18 SiA4) and LN18 α₉β₁ (LN18 α₉β₁SiA4) were negatively selected for α₄ expression using FACS.

(iii) Immunohistochemistry

### (a) Antibody Staining.

LN18 SiA4 (control cell line), LN18 α₄β₁, and LN18 α₉β₁ cells were stained with 2.5 μg ml⁻¹ of mouse-anti-human α₄ antibody (BD Bioscience), 4 μg ml⁻¹ of mouse-anti-human α₉β₁ antibody (Millipore) or 4 μg ml⁻¹ of mouse isotype control (BD Bioscience) in PBS-2% FBS for one hour, followed by 5 μg ml⁻¹ of Alexa Fluor 594 conjugated goat-anti-mouse IgG1 for 1 h and then washed with PBS-2% FBS three times.

### (b) Antibody Cocktails.

For analysis of R-BC154 (IXb) binding to murine progenitor cells (LSK; Lineage⁻Sca-1⁺c-kit⁺) and HSC (LSKSLAM; LSKCD150⁺CD48⁻), BM and PB cells were immunolabelled with a lineage cocktail (anti-Ter119, anti-B220, anti-CD3, anti-Gr-1, anti-Mac-1), anti-Sca-1, anti-c-kit, anti-CD48 and anti-CD150. For lineage analysis, cells were stained separately for T-cells using anti-CD3, B-cells using anti-B220, macrophages using anti-Mac-1 and granulocytes using anti-Gr-1. Alternatively, lineage analysis was also performed using a cocktail containing anti-CD3/B220 (PB conjugated) and anti-B220/Gr1/Mac-1 (AF647 conjugated), whereby B220⁺ cells were identified as +/+ cells, CD3⁺ cells are +/− and Gr1/Mac-1⁺ cells are −/+ populations. For analysis of human WBC from cord blood MNCs or BM and PB from humanised NSG mice, cells were immunolabelled with a lineage cocktail containing anti-huCD3/CD14/CD15 (all AF488 conjugated), anti-CD14/CD15/CD19/CD20 (all AF647 conjugated), anti-huCD45-PB, anti-muCD45-BV510, anti-huCD34-PECy7, anti-huCD34 and anti-huCD38 antibodies. A full list of conjugated antibodies used is detailed in Table 1 and 2.

TABLE 1
Anti-mouse antibodies

| Antibody | Conjugate | Clone | Isotype | Supplier |
|---|---|---|---|---|
| CD3e | FITC | 17A2 | rat IgG2b | Pharmingen |
| CD3e | Biotin | 145-2C11 | hamster | Pharmingen |
| | | | IgG | |
| CD3e | PB | 17A2 | rat IgG2b | Biolegend |
| CD3e | APC/Cy7 | 17A2 | rat IgG2b | Pharmingen |
| CD3e | APC/Cy7 | 17A2 | rat IgG2b | Biolegend |
| CD4, L3T4 | FITC | GK1.5 | rat IgG2b | Pharmingen |
| CD4, L3T4 | PE | GK1.5 | rat IgG2b | Pharmingen |
| CD4, L3T4 | PB | GK1.5 | rat IgG2b | Pharmingen |
| CD4, L3T4 | Biotin | GK1.5 | rat IgG2b | Pharmingen |
| CD8a, Ly-2 | Biotin | 53-6.7 | rat IgG2a | Pharmingen |
| CD8a, Ly-2 | FITC | 53-6.7 | rat IgG2a | Pharmingen |
| CD8a, Ly-2 | PE | 53-6.7 | rat IgG2a | Pharmingen |
| CD8a, Ly-2 | APC | 53-6.7 | rat IgG2a | Biolegend |
| CD11b, Mac-1 | AF647 | M1/70 | rat IgG2b | Biolegend |
| CD11b, Mac-1 | FITC | M1/70 | rat IgG2b | Pharmingen |
| CD11b, Mac-1 | PE | M1/70 | rat IgG2b | Pharmingen |
| CD11b, Mac-1 | PB | M1/70 | rat IgG2b | Biolegend |
| CD11b, Mac-1 | APCCy7 | M1/70 | rat IgG2b | Pharmingen |
| CD45 | AF647 | 30-F11 | rat IgG2b | Biolegend |
| CD45 | APC | 30-F11 | rat IgG2b | Pharmingen |
| CD45 | FITC | 30-F11 | rat IgG2b | Pharmingen |
| CD45 | Biotin | 30-F11 | rat IgG2b | Pharmingen |
| CD45 | PB | 30-F11 | rat IgG2b | Biolegend |
| CD45 | PE | 30-F11 | rat IgG2b | Pharmingen |
| CD45 | PE-Cy7 | 30-F11 | rat IgG2b | Biolegend |
| CD45 | V500 | 30-F11 | rat IgG2b | Pharmingen |
| CD45 | BV421 | 30-F11 | rat IgG2a | BD Horizon |
| CD45 | BV510 | 30-F11 | rat IgG2a | BD Horizon |
| CD45 | BV650 | 30-F11 | rat IgG2a | BD Horizon |
| CD45 | APCCy7 | 30-F11 | rat IgG2a | BD Horizon |
| CD45R, B220 | Biotin | RA3_6B2 | rat IgG2a | Pharmingen |
| CD45R, B220 | FITC | RA3_6B2 | rat IgG2a | Pharmingen |
| CD45R, B220 | PB | RA3_6B2 | rat IgG2a | Biolegend |
| CD45R, B220 | PE | RA3_6B2 | rat IgG2a | Pharmingen |
| CD45R, B220 | AF647 | RA3_6B2 | rat IgG2a | Pharmingen |
| CD45R, B220 | APCCy7 | RA3_6B2 | rat IgG2a | Pharmingen |
| CD45R, B220 | V500 | RA3_6B2 | rat IgG2a | Pharmingen |
| CD45R, B220 | BV850 | RA3_6B2 | rat IgG2a | Biolegend |
| CD45R, B220 | BV650 | RA3_6B2 | rat IgG2a | BD Horizon |
| CD48 | Biotin | HM48-1 | A. hamster | Biolegend |
| | | | IgG | |
| CD48 | FITC | HM48-1 | A. hamster | Pharmingen |
| | | | IgG1 | |
| CD48 | FITC | HM48-1 | A. hamster | Biolegend |
| | | | IgG1 | |
| CD48 | PB | HM48-1 | A. hamster | Biolegend |
| | | | IgG | |
| CD48 | BV421 | HM48-1 | A. hamster | BD horizon |
| | | | IgG1 | |
| CD48 | APC | HM48-1 | A. hamster | BD |
| | | | IgG | Pharmingen |
| CD117, c-kit | AF647 | 2B8 | rat IgG2b | Biolegend |
| CD117, c-kit | APC | 2B8 | rat IgG2b | Pharmingen |
| CD117, c-kit | Biotin | 2B8 | rat IgG2b | Pharmingen |
| CD117, c-kit | FITC | 2B8 | rat IgG2b | Pharmingen |
| CD117, c-kit | PE | 2B8 | rat IgG2b | Pharmingen |
| CD150 | Biotin | TC15-12F12.2 | rat IgG2a | Biolegend |
| (SLAM) | | | | |
| CD150 | PB | TC15-12F12.2 | rat IgG2a | Biolegend |
| (SLAM) | | | | |
| CD150 | PE | TC15-12F12.2 | rat IgG2a | Biolegend |
| (SLAM) | | | | |
| CD150 | BV421 | TC15-12F12.2 | rat IgG2a | Biolegend |
| (SLAM) | | | | |
| CD150 | BV650 | TC15-12F12.2 | rat IgG2a | Biolegend |
| (SLAM) | | | | |
| GR-1, Ly-6G | AF647 | RB6-8C5 | rat IgG2b | Biolegend |
| GR-1, Ly-6G | APCCy7 | RB6-8C5 | rat IgG2b | Pharmingen |
| GR-1, Ly-6G | Biotin | RB6-8C5 | rat IgG2b | Pharmingen |
| GR-1, Ly-6G | FITC | RB6-8C5 | rat IgG2b | Pharmingen |
| GR-1, Ly-6G | PE | RB6-8C5 | rat IgG2b | Pharmingen |
| GR-1, Ly-6G | PB | RB6-8C5 | rat IgG2b | Biolegend |
| Sca-1, Ly-6A/E | Biotin | E13-161.7 | rat IgG2a | Pharmingen |
| Sca-1, Ly-6A/E | FITC | E13-161.7 | rat IgG2a | Pharmingen |
| Sca-1, Ly-6A/E | PB | E13-161.7 | rat IgG2a | Biolegend |
| Sca-1, Ly-6A/E | PE | E13-161.7 | rat IgG2a | Pharmingen |
| Sca-1, Ly-6A/E | PE | D7 | rat IgG2a | Pharmingen |
| Sca-1, Ly-6A/E | BV421 | D7 | rat IgG2a | Pharmingen |
| Sca-1, Ly-6A/E | PECy7 | E13-161.7 | rat IgG2a | Biolegend |
| Sca-1, Ly-6A/E | APC | E13-161.7 | rat IgG2a | Biolegend |
| TER119 | APC | TER119 | rat IgG2b | Pharmingen |
| TER119 | Biotin | TER119 | rat IgG2b | Pharmingen |
| TER119 | FITC | TER119 | rat IgG2b | Pharmingen |
| TER119 | PE | TER119 | rat IgG2b | Pharmingen |
| CD45 | BV650 | 30-F11 | Rat IgG2a | BD Horizon |

TABLE 2
Anti-human antibodies

| Antibody | Conjugate | Clone | Isotype | Supplier |
|---|---|---|---|---|
| CD3 | AF647 | OKT3 | Mouse IgG2a | BioLegend |
| CD14 | AF488 | M5E2 | Mouse IgG2a | BioLegend |
| CD14 | AF488 | M5E2 | Mouse IgG2a | BD Biosciences |
| CD15 | AF488 | H198 | Mouse IgM | BioLegend |
| CD19 | AF488 | HIB19 | Mouse IgG1 | BioLegend |
| CD19 | AF647 | HIB19 | Mouse IgG1 | BioLegend |
| CD20 | AF488 | 2H7 | Mouse IgG2b | BioLegend |
| CD20 | AF647 | 2H7 | Mouse IgG2b | BioLegend |
| CD34 | FITC | 8G12 | Mouse IgG1 | BD Biosciences |
| CD34 | PECy7 | 8G12 | Mouse IgG1 | BD Biosciences |
| CD38 | PECy7 | HB7 | Mouse IgG1 | BD Biosciences |
| CD45 | PB | HI30 | Mouse IgG1 | Biolegend |
| CD45 | BV650 | HI30 | Mouse IgG1 | BD Biosciences |
| CD45 | PE | J.33 | Mouse IgG1 | Immunotech |

### (c) R-BC154 (IXb) Staining

Cultured LN18 SiA4 (control cell line), LN18 α₄β₁, and LN18 α₉β₁ cells were treated with R-BC154 (IXb) (50 nM) in TBS-2% FBS (50 mM TrisHCl, 150 mM NaCl, 2 mM glucose, 10 mM Hepes, pH 7.4) containing 1 mM CaCl₂—MgCl₂ or 1 mM MnCl₂) and incubated for 20 min at 37° C. and then washed with TBS-2% FBS three times. The stained cells were fixed with 4% paraformaldehyde in PBS for 5 min, washed with water three times and then stained with 2.5 μg ml⁻¹ of DAPI. The cells were mounted in Vectorshield, washed with water, coverslipped and stored at 4° C. overnight before images were taken under fluorescent microscope (Olympus BX51).

(iv) Saturation Binding Experiments

Cultured α₄β₁, α₉β₁ and control LN18 cells (0.5×106 cells) were treated with 100 μl of compound (R-BC154) at 0, 1, 3, 10, 30 and 100 nM in TBS-2% FBS (containing either no cations, 1 mM CaCl₂—MgCl₂ or 1 mM MnCl₂). The cells were incubated at 37° C. for 60 min, washed once with TBS-2% FBS, dry pelleted and resuspended in the relevant binding buffer for flow cytometric analysis. Mean channel fluorescence was plotted against concentration and fitted to a one-site saturation ligand binding curve using GraphPad Prism 6. The dissociation constant, K_{d} was determined from the curves.

(v) Off-Rate Kinetics Measurements

Eppendorf vials containing α₄β₁ or α₉β₁ LN18 cells (0.5×10⁶ cells) were treated with 50 nM of R-BC154 (IXb) (100 μl in TBS-2% FBS containing either 1 mM CaCl₂—MgCl₂ or 1 mM MnCl₂ at 37° C. until for 30 min, washed once with the relevant binding buffer and dry pelleted. The cells were treated with 500 nM of an unlabelled competing inhibitor (100 μl, in TBS-2% FBS containing either 1 mM CaCl₂—MgCl₂ or 1 mM MnCl₂) at 37° C. for the times indicated (0, 2.5, 5, 15, 30, 45, 60 min). The cells were diluted with cold TBS-2% FBS (containing the relevant cations), pelleted by centrifugation, washed once and resuspended (˜200 μl) in binding buffer for flow cytometric analysis. Mean channel fluorescence was plotted against time and the data was fitted to either a one-phase or two-phase exponential decay function using GraphPad Prism 6. The off-rate, k_{off} was extrapolated from the curves.

(vi) On-Rate Kinetics Measurements

Eppendorf vials containing α₄β₁ or α₉β₁ LN18 cells (0.5×10⁶ cells) in 50 μl TBS-2% FBS containing either 1 mM CaCl₂—MgCl₂ or 1 mM MnCl₂ were pre-activated in a heating block for 20 min at 37° C. 100 nM R-BC154 (IXb) (50 μl−final concentration=50 nM) in the relevant TBS-2% FBS (with relevant cations) was added to each tube and after 0, 0.5, 1, 2, 3, 5, 10, 15 and 20 min incubation at 37° C., the tubes were quenched by the addition of 3 ml of TBS-2% FBS (with relevant cations). The cells were washed once TBS-2% FBS (with relevant cations), pelleted by centrifugation and resuspended (200 μl) in the relevant binding buffer for flow cytometric analysis. Mean channel fluorescence was plotted against time and the data was fitted to either a one-phase or two phase association function using GraphPad Prism 6. The observed on-rate, k_{obs} was extrapolated from the curves and kₒₙ was calculated using

(k_{obs}−k_{off})/[R-BC154(IXb)=50 nM].

(vii) Mice

C57BL/6 mice were bred at Monash Animal Services (Monash University, Clayton, Australia). Mice were 6-8 weeks old and sex-matched for experiments.

C57BL/6 (C57), RFP, GFP and α₄^{flox/flox}/α₉^{flox/flox} vav-cre mice were bred at Monash Animal Services. Red fluorescent protein (RFP) mice were provided by Children's Medical Research Institute, Sydney, Australia. Conditional α₄^{flox/flox}/α₉^{flox/flox} mice were initially generated by cross breeding α₄^{flox/flox} mice (gift from University of Washington, Department of Medicine/Hematology) with as α₉^{flox/flox} mice (Department of Medicine, University of California) and vav-cre mice (WEHI Institute, Melbourne). NODSIL2Rγ^{-/-} (NSG) mice were obtained in-house (Australian Regenerative Medicine Institute). Humanised NSG mice were generated by tail vein injection of freshly sorted cord blood CD34⁺ cells (>150 k) with 2×10⁶ irradiated mononuclear support cells. After 4-weeks post-transplantation, NSG mice were eyebled and assessed huCD45 and muCD45, and CD34 engraftment. For transplants, irradiation was given in a split dose (5.25 Gy each) 6 hours apart, 24 h before transplant for C57BL/6 mice and in a single dose (2.75 Gy) 5 hours prior to transplant for NSG mice and a total of 2×105 irradiated (15 Gy) C57BL/6 BM cells or 2×106 irradiated (15 Gy) umbilical cord blood (CB) mononuclear cells (MNC) given as support cells to each recipient, respectively.

(viii) In Vivo Bone Marrow Binding Assay

R-BC154 (IXb) in PBS (10 mg kg-1) was injected intravenously into C57 mice. After 5 min, bone marrow cells were isolated as previously described in D. N. Haylock et at Stem Cells, 2007, 25, 1062-1069 and J. Grassinger, et al Cytokine, 2012, 58, 218-225. Briefly, one femur, tibia and iliac crest were excised and cleaned of muscle. After removing the epi- and metaphyseal regions, bones were flushed with PBS-2% FBS to obtain whole bone marrow, which were washed with PBS-2% FBS and then immunolabelled for flow cytometry. For analysis of R-BC154 (IXb) binding, the following antibody combinations were chosen to minimise emission spectra overlap. For staining progenitor cells (LSK; Lineage-Sca-1⁺c-kit⁺) and HSC (LSKSLAM; LSKCD150+CD48−), cells were labelled with a lineage cocktail (CD3, Ter-119, Gr-1, Mac-1, B220; all antibodies APC-Cy7 conjugated), anti-Sca-1-PB, anti-c-kit-AF647, anti-CD48-FITC and anti-CD150-BV650.

(ix) Hemopoietic Cell Isolation.

Populations of endosteal and central murine bone marrow cells were isolated as previously described in J. Grassinger, et al Cytokine, 2012, 58, 218-225 and D. N. Haylock et al Stem Cells, 2007, 25, 1062-1069. Briefly, one femur, tibia and iliac bone were excised and cleaned of muscle. After removing the epi- and metaphyseal regions, bones were flushed with PBS-2% FBS to obtain central bone marrow cells. Flushed long bones and epi- and metaphyseal fragments were pooled and crushed using a mortar and pestle. Bone fragments were digested with Collagenase I (3 mg/ml) and Dispase II (4 mg/ml) at 37° C. in an orbital shaker at 750 rpm. After 5 min, bone fragments were washed once with PBS and once with PBS 2% FBS to collect the endosteal bone marrow cells. Peripheral blood was collected by retro-orbital puncture and red blood cells were lysed using NH₄Cl lysis buffer for 5 min at room temperature. Isolated cell populations were washed with PBS 2% FBS and then stained for flow cytometry as described in Antibody Cocktails above.

(x) Isolation of Human CD34⁺ Cells

Mononuclear cells (MNC) were isolated from cord blood as previously described in Nilsson, S. K. et al Blood 106, 1232-1239, (2005) and Grassinger, J. et al. Blood 114, 49-59, (2009). MNCs were incubated with a lineage antibody cocktail containing mouse anti-human CD3, CD11b, CD14, CD16, CD20, CD24, and CD235a (BD) and then treated with two rounds of DynaI sheep anti-mouse IgG beads (Invitrogen, Carlsbad, Calif.) at a ratio of 2 beads per cell for 5 min and then 10 min at 4° C. with constant rotation. Enriched MNC were stained with CD34-fluorescein isothiocyanate (FITC) CD34 cells purified by FACS.

(xi) In Vitro and In Vivo R-BC154 (IXb) Binding.

For in vitro labelling experiments, 5×10⁶ BM cells from C57 mice, conditional α₄^{-/-}/α₉^{-/-} mice and humanised NODSCIDIL2Rγ^{-/-} mice and human cord blood MNCs were treated with R-BC154 (IXb) (up to 300 nM) in PBS (0.5% BSA) or TBS (0.5% BSA) containing either 1 mM CaCl₂/MgCl₂ (activating) or 10 mM EDTA (deactivating) at 40×10⁶ cells/ml for 20 mins at 4° C. Cells were washed with cold PBS (2% FBS) and then immunolabelled as described in “Antibody Cocktails” prior to flow cytometric analysis. For in vivo experiments, C57BL/6 mice, α₄^{-/-}/α₉^{-/-} vav-cre mice and humanised NODSCIDIL2Rγ^{-/-} mice received either intravenous or subcutaneous injections of R-BC154 (IXb) (5-10 mg/kg) at 100 ul/10 gm mouse weight and analysed as described above.

R-BC154 (IXb) binding analysis on sorted populations of progenitor cells (LSK cells) by fluorescence microscopy were performed wherein, BM cells harvested from untreated and R-BC154 (IXb) injected mice were lineage depleted for B220, Gr-1, Mac-1 and Ter-119, stained with anti-Sca-1-PB and anti-c-kit-FITC and sorted on Sca1⁺c-kit⁴. Sorted cells were imaged using an Olympus BX51 microscope.

(xii) Competitive Inhibition Assays.

α₄β₁ and α₉β₁ LN18 cells (1-2×10⁵ cells) were treated with 50 nM of R-BC154 (IXb) (80 μl in PBS-2% FBS containing 1 mM CaCl₂/MgCl₂) at 37° C. for 10 mins, washed with PBS, pelleted by centrifugation and then treated with BOP (80 μl, PBS-2% FBS containing 1 mM CaCl₂/MgCl₂) at 0, 0.01, 0.1, 0.3, 1, 10, 100 and 300 nM. Cells were incubated for 90 min at 37° C., washed with PBS, pelleted by centrifugation and resuspended in PBS (200 μl) for flow cytometric analysis. % Max mean fluorescence intensity (MFI) was plotted against the log concentration of BOP and the data fitted to a ligand binding-sigmoidal dose-response curve and IC₅₀ values obtained from graphs. For competitive displacement of R-BC154 (IXb) binding to LSK and LSKSLAM cells, WBM cells isolated from mice injected with R-BC154 (IXb) were treated with 500 nM BOP in PBS (containing 0.5% BSA and 1 mM CaCl₂/MgCl₂) for 45 mins at 37° C. prior to flow cytometric analysis.

(xiii) Mobilization Protocols

For mobilization experiments, all mice received subcutaneous injections at 100 μl/10 gm body weight and PB was harvested by throat bleed using EDTA coated syringes.

(a) R-BC154 (IXb) and BOP. Mice received a single injection of freshly prepared solutions of R-BC154 (IXb) and BOP in saline at the doses indicated before PB was harvested by throat bleed at the times indicated.

(b) G-CSF. Mice received G-CSF at 250 μg/kg twice daily (500 ug/kg/day), 6-8 hours apart for 4 consecutive days. Groups receiving G-CSF and BOP received the standard G-CSF regime as described above followed by a single injection of BOP 1 h prior to harvest. Control mice received an equal volume of saline.

(xiv) Mobilization of Humanised NODSIL2Rγ (NSG) Mice

Humanised NSG mice were generated by tail vein injection of freshly sorted cord blood CD34⁺ cells (>150 k) with 2×10⁶ irradiated mononuclear support cells. After 4-weeks post-transplantation, NSG mice were eyebled and assessed for huCD45 and muCD45. Under these conditions, >90% humanisation was achieved as determined by flow cytometric analysis based on % huCD45 relative to total % CD45. Humanised NSG mice were given at least 1 week to recover prior to experimentation. Mice were mobilized under the relevant conditions specified in “Mobilization protocols” and PB subsequently collected by throatbleed, lysed and immunolabelled as described in “Antibody cocktails”.

(a) HSC Mobilization.

Mice received subcutaneous injections at 100 μl/10 gm body weight of a single injection of BOP (up to 15 mg/kg) for various timeframes, a single BIO5192 injection at 1 mg/kg for 1 hour, a single AMD3100 injection at 3 mg/kg for 1 hour (mice also receiving BOP or BIO15192 were injected with a single dose of BOP or BIO15192 1 hour prior to harvest), or G-CSF at 250 μg/kg twice daily (500 μg/kg/day), 6-8 hours apart for 4 consecutive days (mice also receiving BOP and/or AMD3100 were injected with a single dose of BOP and/or AMD3100 1 h prior to harvest). Control mice received an equivalent volume of saline or 10% HPβCD/saline where appropriate.

(xv) Low- and High-Proliferative Potential Colony-Forming Cell Assays

Low- and high-proliferative potential colony-forming cells (LPP-CFC and HPP-CFC, respectively) were assayed as previously described in J. Grassinger et al Cytokine, 2012, 58, 218-225 and Bartelmez, S. H. et al Experimental hematology 17, 240-245 (1989). Briefly, mobilized PB were lysed and 4000 WBCs were plated in 35 mm Petri dishes in a double-layer nutrient agar culture system containing recombinant mouse stem cell factor and recombinant human colony-stimulating factor-1, interleukin-1α (IL-1α), and IL-3. Cultures were incubated at 37° C. in a humidified incubator at 5% O₂, 10% CO₂, 85% N₂. LPP-CFC and HPP-CFC were enumerated at 14 days of incubation as previously described in J. Grassinger, et al (2012).

(xvi) Long-Term Transplant Assays

(a) Limiting Dilution Analysis.

RFP mice were treated with either BOP (n=15), AMD3100 (n=5) or a combination of BOP and AMD3100 (n=5) and PB harvested after 1 h. PB from each donor mouse per treatment group were pooled, lysed and taken up at ⅓ of the original blood volume in PBS. Irradiated WBM filler cells (2×10⁵/mouse) were added to aliquots of lysed PB at the specified transplant volume and then topped up with PBS to allow 200 μl injection/mouse. Irradiated C57BL/6 mice were administered by tail vein injection and multi-lineage RFP engraftment assessed at 6, 12 and 20 weeks post-transplant.

(b) Competitive Primary and Secondary Transplant Assay.

RFP (n=5) and GFP (n=5) mice were treated with BOP/AMD3100 (1 h) and G-CSF (twice daily for 4 d), respectively as described in “Mobilization protocols”. PB was then harvested and blood within RFP and GFP groups were pooled, lysed, washed and resuspended to ⅓ of the original blood volume in PBS. Equal volumes of RFP and GFP blood were mixed to allow transplantation of 500 μl of RFP and GFP blood per mouse with. Irradiated WBM filler cells (2×105/mouse) were added and the mixture topped up in PBS to allow 200 μl injection/mouse. Irradiated C57BL/6 recipients (n=5) were administered by tail vein injection and RFP and GFP engraftment assessed at 6, 12 and 20 weeks post-transplant. At 20 weeks takedown, WBM cells ( 1/10th of a femur) from each primary recipient (n=5) was transplanted into irradiated C57 secondary recipients (n=4/primary recipient) and assessed for multi-lineage engraftment at 6, 12 and 20 weeks post-transplant.

(xvii) Expression of α4 and α931 Integrins on Human HSC.

The expression of human α4 (CD49d) and α9β1 on human HSC from CD34+ enriched human BM cells, BM from huNSG mice and CB MNC CD34+ cells was assessed by the sequential labelling of cells with purified mouse-anti-human α9β1, goat-anti-mouse-AF647 followed by a cocktail of mouse-anti-huCD49d-PECy7, anti-huCD34-FITC and anti-huCD38-BV421. Matching mouse IgG1 isotypes were used as controls.

(xviii) Statistical Analysis

Data were analyzed using student's t-test, one-way or two-way ANOVA where appropriate for the data set. For determination of stem cell repopulation frequency, Poisson analysis using L-CALC software (Stem Cell Technologies) was performed. Log-rank (Mantel-Cox) test was used to compare survival curves. p<0.05 was considered significant.

### Example 1: Preparation of α₉β₁ Integrin Antagonists

(a) Synthesis of Antagonist Compounds

The agents of the various embodiments may be prepared using the reaction routes and synthesis schemes as described below. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3rd Edition, John Wiley & Sons, 1991. Alternatively, other reactions disclosed herein or known in the art will be recognized as having applicability for preparing other compounds of the various embodiments.

Reagents useful for synthesizing compounds may be obtained or prepared according to techniques known in the art.

The symbols, abbreviations and conventions in the processes, schemes, and examples are consistent with those used in the contemporary scientific literature. Specifically but not meant as limiting, the following abbreviations may be used in the examples and throughout the specification.

Unless otherwise indicated, all temperatures are expressed in ° C. (degree centigrade). All reactions conducted at room temperature unless otherwise mentioned.

All starting materials, reagents, and solvents were obtained from commercial sources and used without further purification unless otherwise stated. N-(Benzyloxycarbonyl)-L-prolyl-L-O-(tert-butylether)tyrosine methyl ester 26 was obtained from Genscript. All anhydrous reactions were performed under a dry nitrogen atmosphere. Diethyl ether, dichloromethane, tetrahydrofuran and toluene were dried by passage through two sequential columns of activated neutral alumina on the Solvent Dispensing System built by J. C. Meyer and based on an original design by Grubbs and co-workers. Petroleum spirits refers to the fraction boiling at 40-60° C. Thin layer chromatography (TLC) was performed on Merck pre-coated 0.25 mm silica aluminium-backed plates and visualised with UV light and/or dipping in ninhydrin solution or phosphomolybdic acid solution followed by heating. Purification of reaction products was carried out by flash chromatography using Merck Silica Gel 60 (230-400 mesh) or reverse phase C18 silica gel. Melting points were recorded on a Reichert-Jung Thermovar hot-stage microscope melting point apparatus. Optical rotations were recorded on a Perkin Elmer Model 341 polarimeter. FTIR spectra were obtained using a ThermoNicolet 6700 spectrometer using a SmartATR (attenuated total reflectance) attachment fitted with a diamond window. Proton (¹H) and carbon (¹³C) NMR spectra were recorded on a BrukerAV400 spectrometer at 400 and 100 MHz, respectively. ¹H NMR are reported in ppm using a solvent as an internal standard (CDCl₃ at 7.26 ppm). Proton-decoupled ¹³C NMR (100 MHz) are reported in ppm using a solvent as an internal standard (CDCl₃ at 77.16 ppm). High resolution mass spectrometry was acquired on either a WATERS QTOF II (CMSE, Clayton, VIC 3168) or a Finnigan hybrid LTQ-FT mass spectrometer (Thermo Electron Corp., Bio21 Institute, University of Melbourne, Parkville, VIC 3010) employing Electrospray Ionisation (ESI).

### Example 1A: Preparation of N-(Benzenesulfonyl)-L-prolyl-L-O-(1-pyrrolidinyl carbonyl)tyrosine (BOP)

Synthesis of BOP began from the dipeptide 26, as shown in the following Scheme 1:

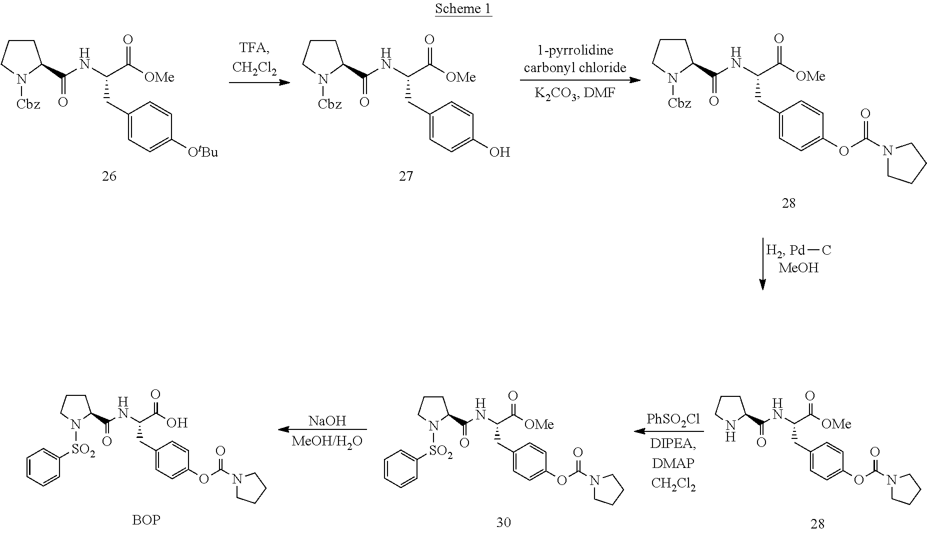

Deprotection of the tert-butyl protecting group of 26 using trifluoroacetic acid at 0° C. provided phenol 27, which was used in the next step, after aqueous work-up, without further purification. Reaction of phenol 27 with 1-pyrrolidinecarbonyl chloride proceeded smoothly in the presence of potassium carbonate to provide carbamate 28 in good yield (74%) over two steps. Hydrogenolysis of the Cbz protecting group was complete within 3 hours, and the resulting amine was obtained in excellent yield (85%) after flash chromatography. Amine 29 was then reacted with benzenesulfonyl chloride in the presence of base to give the sulfonamide 30 in excellent yield (96%) after flash chromatography. Finally, the methyl ester moiety of 30 was saponified using sodium hydroxide, followed by ion-exchange on Amberlyst resin, to provide BOP in 81% yield after flash chromatography.

By way of exemplification the actual reaction conditions for the formation of BOP, starting from dipeptide 26 is provided herein.

### Step 1: N-(Benzyloxycarbonyl)-L-prolyl-L-O-tyrosine methyl ester (27)

Trifluoroacetic acid (TFA) (1.27 mL, 16.6 mmol) was added dropwise to a suspension of N-(benzyloxycarbonyl)-L-prolyl-L-O-(tert-butylether)tyrosine methyl ester 26 (0.80 g, 1.66 mmol; custom peptide synthesis from Genscript) in dry CH₂Cl₂ (10 mL) at 0° C. The mixture was slowly warmed to rt and stirred for 3 h at which point TLC (70:30 EtOAc/petroleum spirits) indicated complete consumption of starting material. The mixture was diluted with EtOAc and washed with H₂O, brine, dried (MgSO₄) and concentrated under reduced pressure. The residue was concentrated with toluene (×3) to give the crude N-(benzyloxycarbonyl)-L-prolyl-L-O-tyrosine methyl ester 27 (700 mg) as a colourless oil, which was used in the next step without further purification.

### Step 2: N-(Benzyloxycarbonyi)-L-prolyl-L-O-(1-pyrrolidinylcarbonyl)tyrosine methyl ester (28)

1-Pyrrolidinecarbonyl chloride (147 μL, 1.38 mmol) was added to a mixture of the crude phenol 27 (393 mg, 0.922 mmol) and K₂CO₃ (256 mg, 1.84 mmol) in N, N-dimethylformamide (DMF) (5 mL). The mixture was stirred at 50° C. overnight, diluted with EtOAc/H₂O and the organic phase separated. The organic layer was washed with 5% HCl, sat. aq. NaHCO₃, brine, dried (MgSO₄) and concentrated under reduced pressure. The residue was purified by flash chromatography (70% EtOAc/petroleum spirits) to give the carbamate 28 (355 mg, 74%) as a colourless foam, which was used in the next step without further purification.

### Step 3: L-prolyl-L-O-(1-pyrrolidinylcarbonyl)tyrosine methyl ester (29)

A mixture of the Cbz protected dipeptide 28 (356 mg, 0.681 mmol) and 10% Pd/C (50% H₂O, 150 mg) in MeOH (30 mL) was purged three times with H₂. The mixture was stirred under a H₂ atmosphere for 3 h at which point TLC (10% MeOH/CH₂Cl₂) indicated complete consumption of starting material. The mixture was filtered through a layer of Celite and the filtrate concentrated under reduced pressure. The residue was purified by flash chromatography (5% to 10% MeOH/CH₂Cl₂) to give the amine 29 (224 mg, 85%) as a colourless oil. δ_{H} (400 MHz, CDCl₃) 1.64-1.78 (2H, m), 1.82-1.92 (5H, m), 2.16-2.25 (1H, m), 2.97-3.15 (4H, m), 3.39 (2H, t, J=6.5 Hz), 3.49 (2H, t, J=6.5 Hz), 3.65 (3H, s), 4.03 (1H, dd, J=5.7, 8.3 Hz) 4.72 (1H, dd, J=7.8, 13.3 Hz), 5.69 (1H, br s), 6.99 (2H, d, J=8.3 Hz), 7.13 (2H, d, J=8.3 Hz), 8.41 (1H, d, J=7.9 Hz).

### Step 4: N-(Benzenesulfonyl)-L-prolyl-L-O-(1-pyrrolidinylcarbonyl)tyrosine methyl ester (30)

Diisopropyl ethyl amine (DIPEA) (95 μL, 0.546 mmol) was added to a stirred solution of the amine D (71 mg, 0.182 mmol), PhSO₂Cl (35 μL, 0.273 mmol) and 4-dimethylaminopyridine (DMAP) (2.2 mg, 0.018 mmol) in CH₂Cl₂ (3 mL). The mixture was stirred for 4 h at rt, concentrated under reduced pressure and the residue purified by flash chromatography (2.5% MeOH/CH₂Cl₂) to give the product E (93 mg, 96%) as a colourless foam. δ_{H} (400 MHz, CDCl₃) 1.42-1.56 (3H, m), 1.90-2.05 (5H, m), 3.03 (1H, dd, J=7.6, 14.0 Hz), 3.10-3.16 (1H, m), 3.26 (1H, dd, J=5.6, 14.0 Hz), 3.35-3.40 (1H, m), 3.45 (2H, t, J=6.5 Hz), 3.54 (2H, t, J=6.5 Hz), 3.77 (3H, s), 4.08 (1H, dd, J=2.0, 8.0 Hz), 4.82 (1H, dt, J=5.7, 11.6 Hz), 7.06 (2H, d, J=8.7 Hz), 7.13 (2H, d, J=8.7 Hz), 7.25 (1H, d, J=7.5 Hz; obscured by solvent peak), 7.52-7.57 (2H, m), 7.61-7.65 (1H, m), 7.83-7.85 (2H, m).

### Step 5: N-(Benzenesulfonyl)-L-prolyl-L-O-(1-pyrrolidinylcarbonyl)tyrosine (BOP, Ic)

0.1 M NaOH (3.2 mL, 0.162 mmol) was added to a solution of the ester 30 (86 mg, 0.162 mmol) in MeOH (10 mL) and the mixture stirred overnight at rt. The reaction was quenched with Amberlyst resin (H⁺ form), filtered and the filtrate concentrated under reduced pressure. The crude product was purified by flash chromatography (10% MeOH/CH₂Cl₂) to give the product BOP, Ic (68 mg, 81%) as a colourless glass. δ_{H} (400 MHz, d₄-MeOH) 1.47-1.55 (1H, m), 1.59-1.72 (2H, m), 1.77-1.85 (1H, m), 1.93-2.00 (4H, m), 3.11 (1H, dd, J=7.8, 13.7 Hz), 3.18-3.24 (1H, m), 3.27 (1H, dd, J=5.0, 13.7 Hz), 3.35-3.44 (3H, m), 3.56 (2H, d, J=6.5 Hz), 4.14 (1H, dd, J=4.0, 8.5 Hz), 4.69 (1H, m), 7.04 (2H, d, J=8.5 Hz), 7.27 (2H, d, J=8.5 Hz), 7.60 (2H, t, J=7.6 Hz), 7.69 (1H, t, J=7.4 Hz), 7.86 (2H, d, J=7.4 Hz).

For in vitro and in vivo experiments, BOP (Ic) was converted to the sodium salt by treatment of a solution of the free acid of BOP (Ic) in MeOH with 0.98 equivalents of NaOH (0.01 M NaOH). The solution was filtered through a 0.45 μm syringe filter unit and the product lyophilised to give the sodium salt as a fluffy colourless powder. δ_{H}(400 MHz, D₂O) 1.47-1.59 (2H, m), 1.68-1.83 (2H, m), 1.87-1.92 (4H, m), 3.01 (1H, dd, J=7.7, 13.8 Hz), 3.18-3.26 (2H, m), 3.34-3.40 (3H, m), 3.48-3.51 (2H, m), 4.06 (1H, dd, J=4.4, 8.7 Hz), 4.43 (1H, dd, J=5.0, 7.7 Hz), 7.04 (2H, d, J=8.5 Hz), 7.27 (2H, d, J=8.5 Hz), 7.61 (2H, t, J=8.1 Hz), 7.73 (1H, t, J=7.5 Hz), 7.78 (2H, d, J=7.5 Hz).

### Example 1B: Preparation of R-BC154 (IXb)

Compound IXb (R-BC154), which lacks the PEG-spacer was also synthesised, as shown in the following Scheme 2:

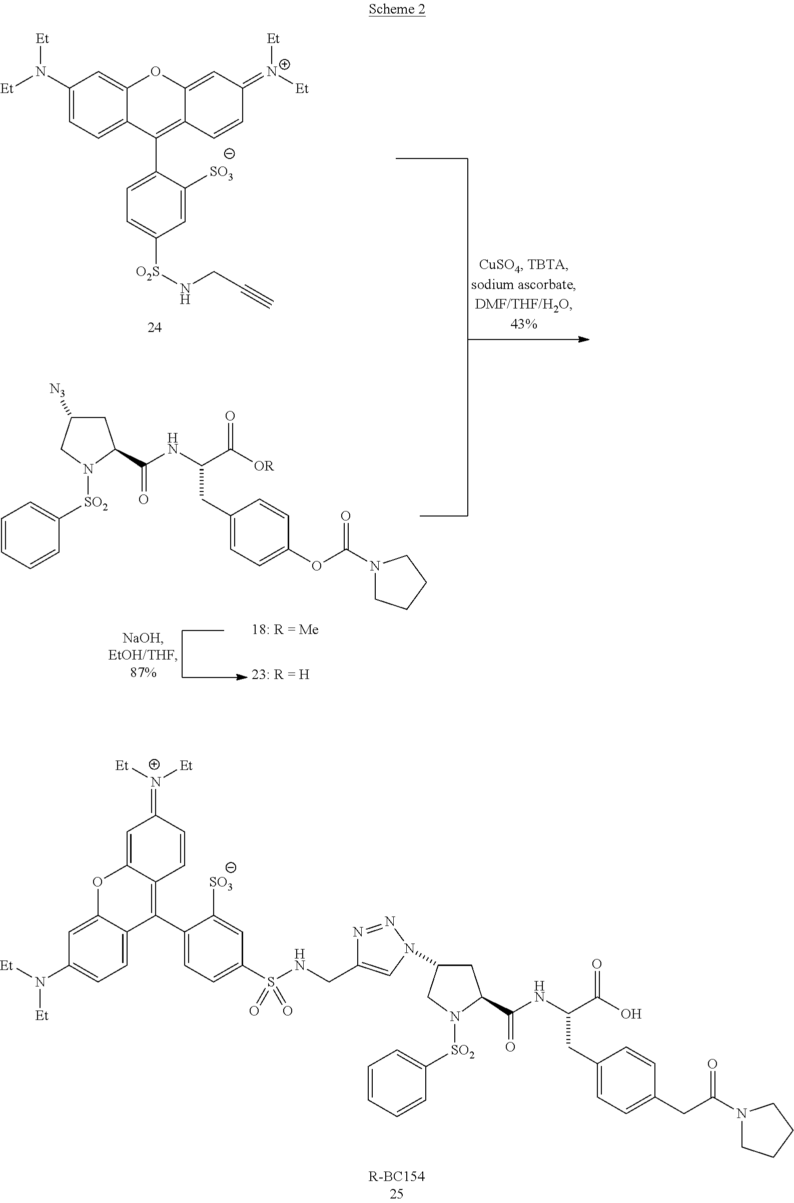

Thus, hydrolysis of the methyl ester 18 with NaOH gave the deprotected azide inhibitor 23, which was subsequently reacted with N-propynyl sulforhodamine B 24 in the presence of CuSO₄, sodium ascorbate and TBTA to give the fluorescent labelled compound of formula IXb (R-BC154) in 43% yield after purification by HPLC.

By way of exemplification the actual reaction conditions for the formation of fluorescently labelled BOP derivative IXb, starting from methyl ester 18 is herein provided.

### Step 1: (S)-2-((2S,4R)-4-Azido-1-(phenylsulfonyl)pyrrolidine-2-carboxamido)-3-(4-((pyrrolidine-1-carbonyl)oxy)phenyl)propanoic acid (23)

The methyl ester 18 (420 mg, 0.737 mmol) in EtOH (10 mL) was treated with 0.2 M NaOH (4.05 mL, 0.811 mmol) and stirred at rt for 1 h. The mixture was concentrated under reduced pressure to remove EtOH and the aqueous phase acidified with 10% HCl. The aqueous phase was extracted with CHCl₃ (4×10 mL) and the combined organic phases were washed with brine, dried (MgSO₄) and concentrated under reduced pressure. The crude material was purified by flash chromatography (10% MeOH/CH₂Cl₂ with 0.5% AcOH) to give acid 23 (384 mg, 94%) as a pale yellow foam, [α]_{D} −0.7 (c 1.00 in CHCl₃); δ_{H} (400 MHz, CDCl₃) 1.67-1.73 (1H, m), 1.89-1.96 (5H, m), 3.10 (1H, dd, J=8.0, 13.8 Hz), 3.21 (1H, dd, J=4.0, 11.5 Hz), 3.38 (1H, dd, J=5.3, 14.0 Hz), 3.44-3.55 (5H, m), 3.81 (1H, m), 4.11 (1H, t, J=6.5 Hz), 4.89 (1H, m), 7.05, 7.22 (4H, 2×d, J=8.0 Hz), 7.41 (1H, d, J=6.8 Hz), 7.53-7.64 (3H, m), 7.85 (2H, d, J=7.5 Hz); δ_{C} (100 MHz, CDCl₃) 25.0, 25.8, 36.1, 36.8, 46.5, 46.6, 53.2, 53.9, 58.9, 61.2, 122.0 (2C), 128.0 (2C), 129.4 (2C), 130.5 (2C), 133.6, 133.7, 136.0, 150.5, 153.6, 170.9, 173.7; v/cm⁻¹ 3329, 2977, 2881, 2105, 1706, 1672; HRMS (ESI) m/z 557.1817 (C₂₅H₂₉N₇O₆S [M+H]⁺ requires 557.1813).

### Step 2: R-BC154 (IXb)

The azide 23 (12 mg, 22 μmol) and N-propynyl sulforhodamine B 24 (14 mg, 24 μmol) in DMF (2 mL) were treated with CuSO₄ (86 μL, 0.86 μmol, 0.01 M in H₂O), sodium ascorbate (430 μL, 4.3 μmol, 0.01 M in H₂O) and tris[(1-benzyl-1H-1,2,3-triazol-4-yl)methyl]amine (TBTA) (108 μL, 1.08 μmol, 0.01 M in DMF). The mixture was stirred at 60° C. for 2 h at which point TLC indicated formation of a new fluorescent product. The mixture was concentrated under reduced pressure and the residue partly purified by flash chromatography (40:10:1 CHCl₃/MeOH/H₂O with 0.5% AcOH). This material was further purified by HPLC (50%-98% MeCN/H₂O (0.1% TFA) gradient over 15 minutes; Rₜ=14.9 min) to give pure IXb (10.6 mg, 43%) as a purple glass, δ_{H} (400 MHz, d₄-methanol) 1.27-1.31 (12H, dt, J=7.0, 3.5 Hz), 1.91-1.98 (4H, m), 2.29-2.35 (1H, m), 2.71-2.78 (1H, m), 3.08 (1H, dd, J=7.5, 13.8 Hz), 3.22 (1H, dd, J=5.3, 13.8 Hz), 3.41 (2H, t, J=6.5 Hz), 3.54 (2H, t, J=6.5 Hz), 3.63-3.70 (8H, m), 3.85 (1H, dd, J=3.5, 12.0 Hz), 3.97 (1H, dd, J=5.6, 11.6 Hz), 4.21 (2H, d, J=1.4 Hz), 4.41 (1H, t, J=7.3 Hz), 4.72 (1H, dd, J=5.4, 7.5 Hz), 5.08 (1H, m), 6.91 (2H, t, J=2.2 Hz), 6.98-7.04 (4H, m), 7.11 (2H, t, J=9.0 Hz), 7.30 (2H, d, J=8.6 Hz), 7.40 (1H, d, J=8.0 Hz), 7.44 (2H, t, J=7.5 Hz), 7.58-7.68 (4H, m), 8.00 (1H, dd, J=1.9, 8.0 Hz), 8.37 (1H, d, J=1.8 Hz); δ_{C} (100 MHz, d₄-methanol) 12.9 (4C), 25.9, 26.7, 37.1, 37.6, 39.0, 46.8 (4C), 47.5, 47.6, 54.8, 55.7, 60.3, 62.1, 97.0 (2C), 115.0 (2C), 115.26, 115.29, 122.9 (2C), 123.9, 127.5, 128.6 (2C), 129.3, 130.5 (2C), 131.6 (2C), 132.3, 133.8, 133.9, 134.4, 135.26, 135.34, 138.3, 144.1, 144.8, 146.9, 151.7, 155.2, 157.16, 157.17, 157.2, 157.8, 159.4, 173.1, 173.8; v/cm⁻¹ 3088-3418, 2977, 2876, 1711, 1649, 1588; HRMS (ESI⁺) m/z 1174.3447 (C₅₅H₆₁N₉NaO₁₃S₃[M+Na]⁺ requires 1174.3443). For in vitro and in vivo testing, the free acid of IXb (11.7 mg, 9.97 μmol) was dissolved in 0.01 M NaOH (997 μL, 9.97 μmol) and the dark purple solution filtered through a 0.45 μm syringe filter unit. The product was lyophilised to give the sodium salt of IXb (11.6 mg, 99%) as a fluffy purple powder.

### Example 2: Analysis of Specific Binding to Human and Murine α₉β₃ on HSC Using R-BC154 (IXb) and BIO5192

In order to assess the specific targeting of α₉β₁ on HSC by small molecule antagonists, an in vitro assay using the selective and potent (Kd<10 pM) α₄β₁ antagonist BIO15192 (Leone, D. R. et al. (2003)), the dual α₉β₁/α₄β₁ antagonist BOP and its fluorescent analogue R-BC154 (IXb) was developed. These efficiently bind to both human and murine (FIG. 1a) α₉β₁ and α₄β₁ integrins in a divalent metal cation dependent manner. This is in contrast to BIO5192, which binds α₄β₁ in the presence and absence of divalent metal cations. The co-labelling of both human and murine α₉β₁/α₄β₁ with R-BC154 (IXb) and excess BIO15192 allowed specific detection of binding to α₉β₁ (FIG. 1b,c). In contrast, R-BC154 (IXb) labelling in combination with excess BOP completely inhibited R-BC154 (IXb) binding to both human and murine α₉β₁/α₄β₁ integrins (FIG. 1b,c). Given the ubiquitous expression of α₄β₁ on all hematopoietic cells, the combination of R-BC154 with BIO5192 provides a convenient method of measuring specific binding to α₉β₁ on hematopoietic stem and progenitors. For the assessment of α₉β₁ contribution to R-BC154 (IXb) binding, human and murine cells were stained with R-BC154 (100 nM) in the absence of competing antagonist or in the presence of excess amounts of the selective α₄β₁ antagonist BIO5192 (1 μM) or dual α₄β₁/α₉β₁ antagonist BOP (1 μM) in PBS containing 0.5% BSA and 1 mM CaCl₂MgCl₂ at 0° C. for 30 min. Cells were washed, immunolabelled as described above and analysed by flow cytometry. The contribution of α₉β₁ binding was calculated by measuring the difference in MFI between R-BC154 (IXb)+BIO5192 and +BOP and expressed as a % of R-BC154 (IXb) alone. Transduced LN18 cells and CHO cells incubated under identical conditions were used as controls, which confirmed BIO5192 inhibited >95% of R-BC154 (IXb) binding to α₄β₁ but did not inhibit binding to α₉β₁. In contrast, BOP inhibited >95% of R-BC154 (IXb) binding to both α₄β₁ and α₉α₁.

### Example 3: R-BC154 (IXb) and BOP Preferentially Bind Human and Murine HSC and Progenitors in a Divalent Cation and α₉β₁ Dependent Manner

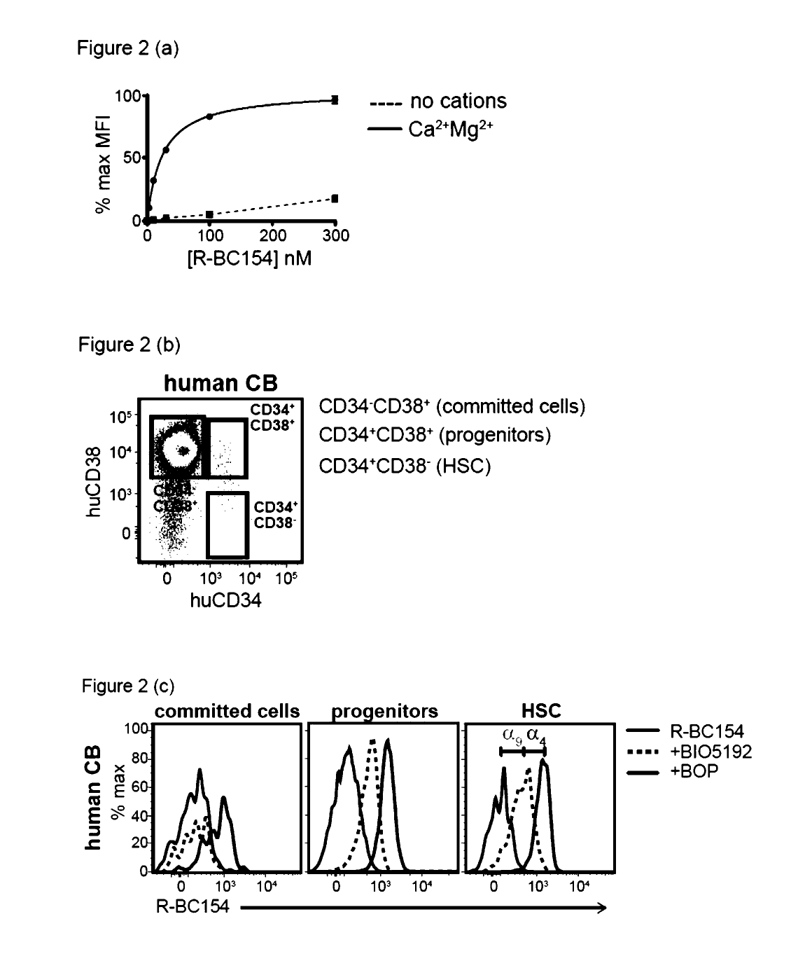

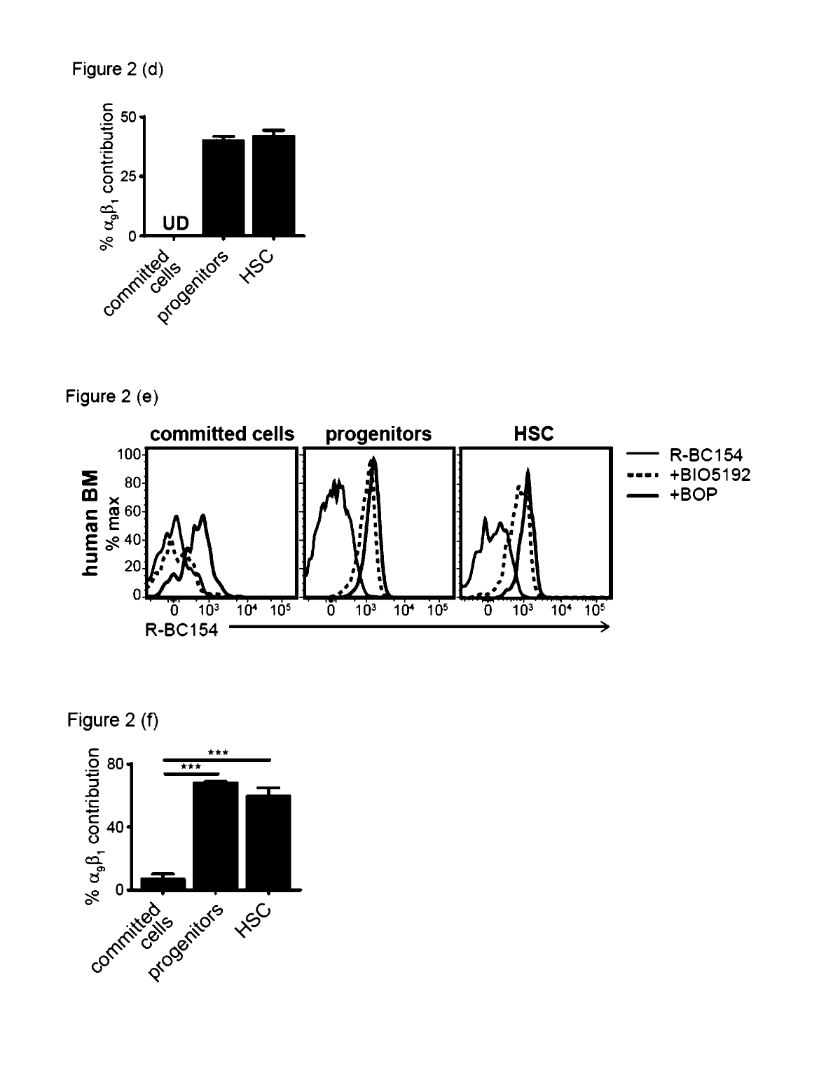

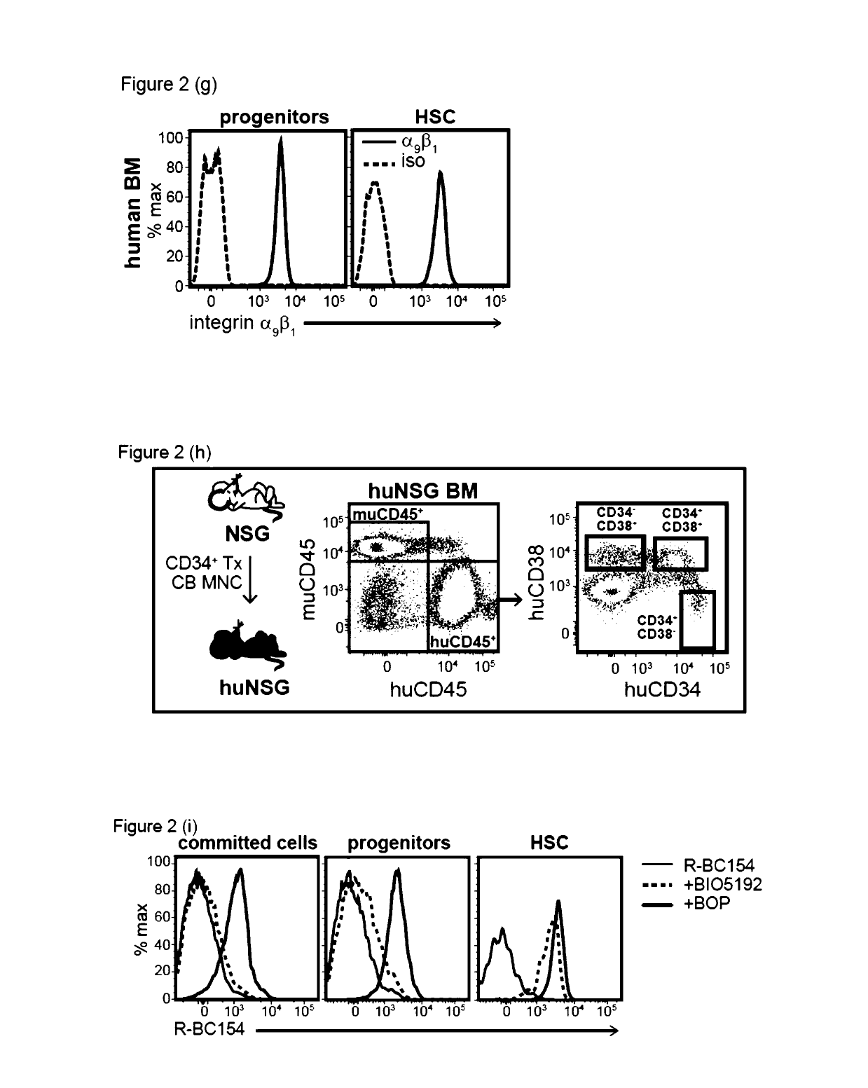

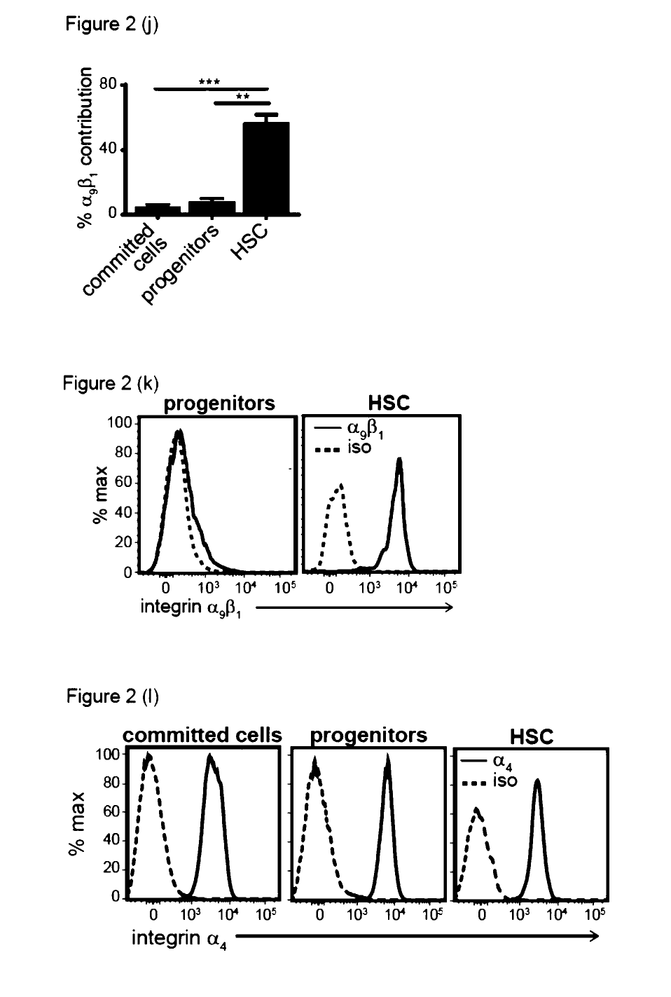

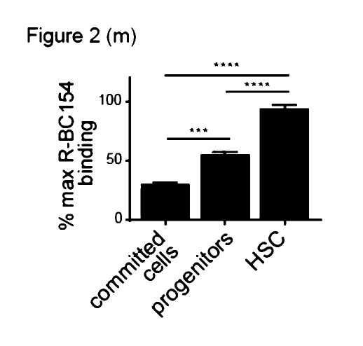

It has previously been demonstrated that human HSC express α₉β₁ and its interaction with trOpn regulates HSC quiescence. To determine whether dual α₉β₁/α₄β₁ or cross reactive antagonists bind human HSC via α₉β₁, R-BC154 (IXb) binding to cord blood (CB) mononuclear cells (MNC) was assessed and shown to be divalent cation and dose dependent, and saturable (FIG. 2a). Analysis of CB HSC (CD34⁺CD38⁻), progenitors (CD34⁺CD38⁺) and lineage committed cells (CD34⁻CD38⁺) (FIG. 2b) revealed high R-BC154 (IXb) binding to HSC and progenitors, but only modest binding to committed cells in the presence of 1 mM Ca²⁺/Mg²⁺ (FIG. 2c). Furthermore, co-incubation of these populations with R-BC154 (IXb) in combination with BIO5192 demonstrated partial inhibition of R-BC154 (IXb) binding to HSC and progenitors (reflective of binding via both α₉β₁ and α₄β₁) and complete inhibition of binding to lineage committed cells (reflective of binding only via α₄β₁) (FIG. 2c). In contrast, as BOP effectively binds both α₉β₁ and α₄β₁, its addition resulted in complete inhibition of R-BC154 (IXb) binding to all cell populations (FIG. 2c). Together, these data demonstrate a significant proportion of R-BC154 (IXb) binding to HSC and progenitors via α₉β₁, while binding to committed cells is mediated exclusively through α₄β₁ (FIG. 2d).

In addition, R-BC154 (IXb) efficiently bound HSC and progenitors isolated from human BM in the presence of Ca²⁺/Mg²⁺ (FIG. 2e) and similarly to that evident with CB cells, the majority of this binding was via α₉β₁ compared to committed cells that primarily bound via α₄β₁ (FIG. 2f). These data are consistent with the high α₉β₁ expression evident on human BM HSC and progenitors (FIG. 2g).

R-BC154 (IXb) binding to human HSC was further evaluated using humanized NODSCIDIL2Rγ^{-/-} (huNSG) mice (FIG. 2h). R-BC154 (IXb) bound human BM committed cells, progenitors and HSC in the presence of Ca²⁺/Mg²⁺ (FIG. 2i), but significant binding through α₉β₁ only occurred on HSC (FIG. 2j), which is consistent with α₉β₁ expression being restricted to HSC (FIG. 2k). In contrast, α₄β₁ was highly expressed on all three populations (FIG. 2l).

The ubiquitous expression of α₄β₁ on hematopoietic cells, together with the restricted expression of α₉β₁ on HSC resulted in an additive R-BC154 (IXb) binding to HSC relative to progenitors and committed cells (FIG. 2m). Together these data highlight the potential for human HSC and/or progenitors to be targeted in preference to lineage committed cells using selective α₉β₁ antagonists.

### Example 4: In Vivo Binding of Compound R-BC154 (IXb) to Bone Marrow HSC and Progenitor Cells

The in vitro binding data demonstrated that R-BC154 (IXb) is a high affinity α₄β₁ and α₉β₁ integrin antagonist, whose binding activity is highly dependent on integrin activation. This example tests whether R-BC154 (IXb) could be used in in vivo binding experiments to investigate α₉β₁/α₄β₁ integrin activity on defined populations of HSC. To date, assessing integrin activity on HSC has relied primarily on in vitro or ex vivo staining of bone marrow cells or purified HSC using fluorescent labelled antibodies. Whilst ex vivo staining provides confirmation of integrin expression by HSC, investigation of integrin activation in their native state within bone marrow can only be determined through in vivo binding experiments, as the complex bone marrow microenvironment cannot be adequately reconstructed in vitro.

To assess whether R-BC154 (IXb) and this class of N-phenylsulfonyl proline-based peptidomimetics could bind directly to HSC, R-BC154 (IXb) (10 mg kg⁻¹) was injected intravenously into mice and analysed for R-BC154 (IXb) labelling of phenotypically defined bone marrow progenitor cells (LSK cell; lineage-Sca-1+c-Kit+) and HSC (LSKSLAM cell; LSKCD48-CD150+) using multi-colour flow cytometry. Increased cell-associated fluorescence as a result of R-BC154 (IXb) binding was observed for both progenitor cells and HSC populations that were isolated from R-BC154 (IXb) injected mice when compared to bone marrow from un-injected mice. Furthermore, in vivo R-BC154 (IXb) binding was also confirmed by fluorescence microscopy on purified populations of progenitor cells (Lineage-Sca-1+c-Kit+). R-BC154 (IXb) labelled progenitor cells exhibited a fluorescence halo indicating R-BC154 (IXb) binding was primarily cell surface, which is consistent with integrin-binding. The in vivo binding results indicate that this class of α₉β₁/α₄β₁ integrin antagonists are capable of binding to extremely rare populations of haemopoietic progenitor cells and HSC, which represent only 0.2% and 0.002% of mononucleated cells within murine bone marrow, respectively.

In these experiments BOP has been shown to inhibit binding of α₉β₁ and α₄β₁ integrins to both VCAM-1 and Opn in vitro with nanomolar inhibitory potencies. These in vivo binding results using R-BC154 (IXb) indicate that the α₄β₁. and α₉β₁ integrins expressed by HSC are in an active binding conformation in situ. This suggests that small molecule α₉β₁/α₄β₁ integrin antagonists such as compound IXb, not only bind directly to bone marrow HSC, but they are also be capable of inhibiting α₉β₁/α₄β₁ dependent adhesive interactions and potentially serve as effective agents for inducing the mobilisation of bone marrow HSC into the peripheral circulation as shown below.

### Example 5: R-BC154 (IXb) Binds Preferentially to Mice and Human Haematopoietic Progenitor Cells In Vitro

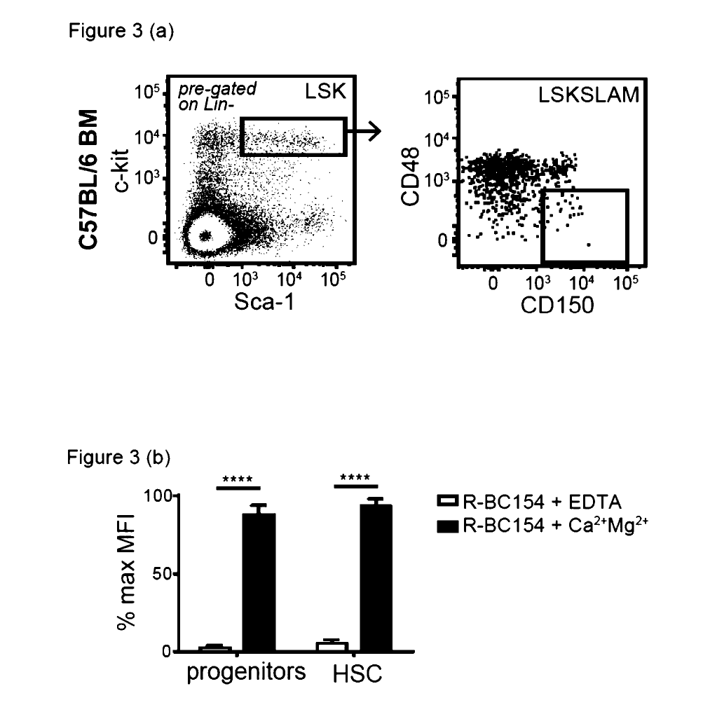

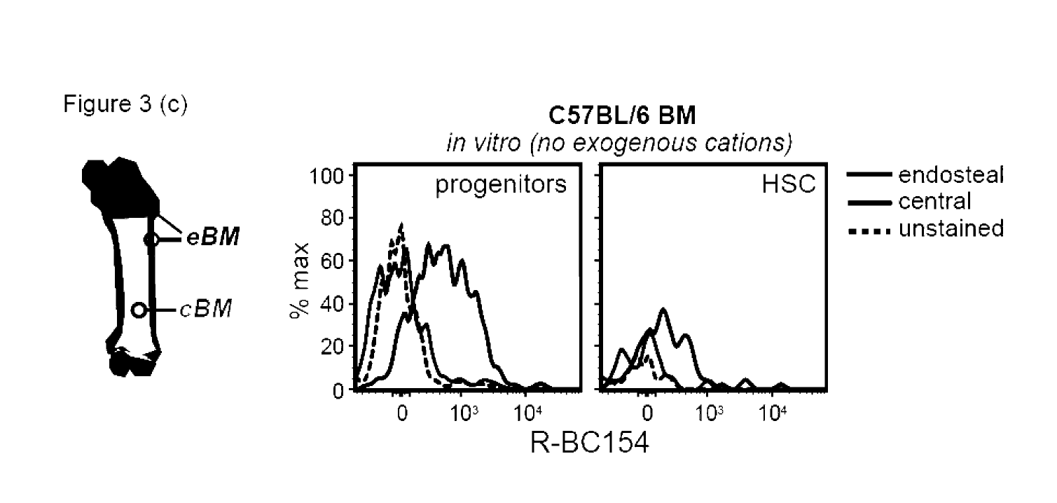

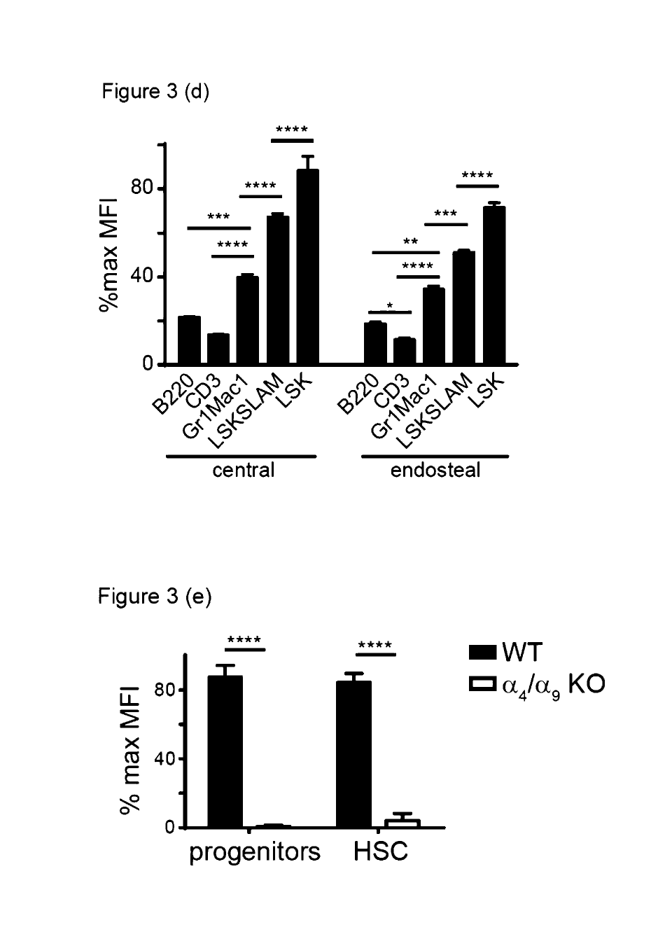

To determine whether integrin activation is required for binding to both central and endosteal BM progenitors (Lin⁻Sca-1⁺ckit⁺ cells; LSK) and HSC (LSKCD150⁺CD48⁻ cells; LSKSLAM) (FIG. 3a), R-BC154 (IXb) binding was assessed in the presence of 1 mM Ca²⁺/Mg²⁺ (FIG. 3b). Under these conditions, greater binding to central LSK and LSKSLAM was observed relative to their endosteal counterparts (p<0.005) (FIG. 3b. Deactivation of surface integrins by co-treatment with EDTA completely abolished activity demonstrating the requirement of integrin activation for efficient R-BC154 (IXb) binding to HSC and progenitors (FIG. 3b). In the absence of both activating cations and EDTA, R-BC154 (IXb) binding to endosteal LSK cells was still evident but not to central LSK (FIG. 3c). These results suggest integrins expressed by HSC and progenitors isolated from the endosteal BM remain activated upon harvest.

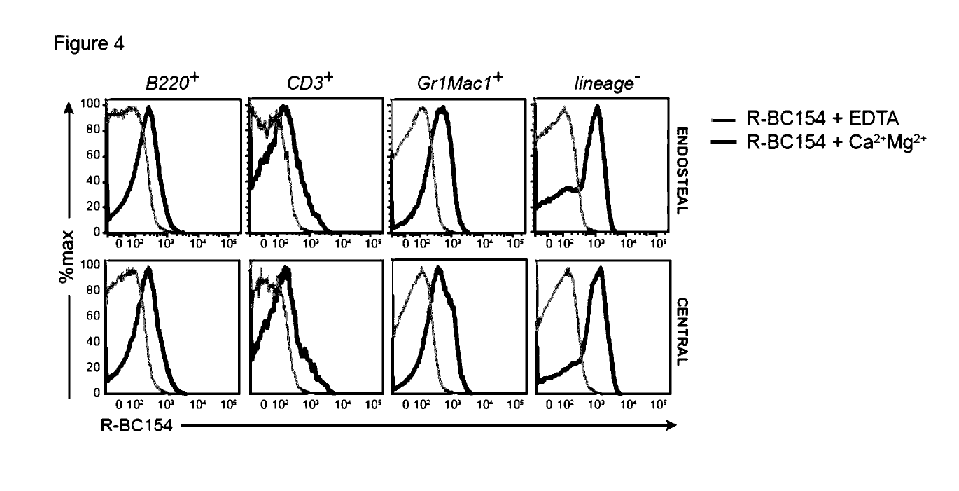

Since integrin α₄β₁ is ubiquitously expressed on all leukocytes and α₉β₁ is known to be widely expressed on neutrophils, R-BC154 (IXb) binding to lineage-committed haematopoietic cells was assessed. It was found that activation dependent binding was observed on all lineage committed lymphoid (B220⁺ and CD3⁺) and myeloid (Gr1/Mac1⁺) progeny isolated from both the central and endosteal BM regions under exogenous activation (FIG. 4). However, this binding was significantly lower relative to LSKSLAM (p<0.0001) and LSK (p<0.0001) cells (FIG. 3d). To confirm whether binding to HSC and progenitor cells is α₄β₁ and α₉β₁ integrin dependent, BM cells devoid of α₄ and α₉ integrins in haematopoietic cells (α₄^{flox/flox}α₉^{flox/flox} vav-cre mice) were treated with R-BC154 (IXb). Binding was essentially absent on LSK (p<0.005) and LSKSLAM (p<0.005) α₄^{-/-}/α₉^{-/-} cells confirming the requirement of these two integrins for R-BC154 (IXb) activity (FIG. 3e).

Divalent cation and dose dependent binding of R-BC154 (IXb) was also confirmed on human cord blood mononuclear cells (MNC) (FIG. 2(a)). Under activating conditions, greater binding was observed on stem cell enriched CD34⁺CD38⁻ cells compared to lineage-committed CD34⁻ cells, albeit to a lesser extent relative to CD34⁺CD38⁺ progenitor cells (FIG. 2 (b)). These results show R-BC154 (IXb) binding to murine and human haematopoietic cells is divalent metal cation dependent and is also biased towards haematopoietic progenitor cells relative to HSC under exogenous activation in vitro.

### Example 6: BOP, but not R-BC154 (IXb), Rapidly and Preferentially Mobilizes HSC and Progenitors

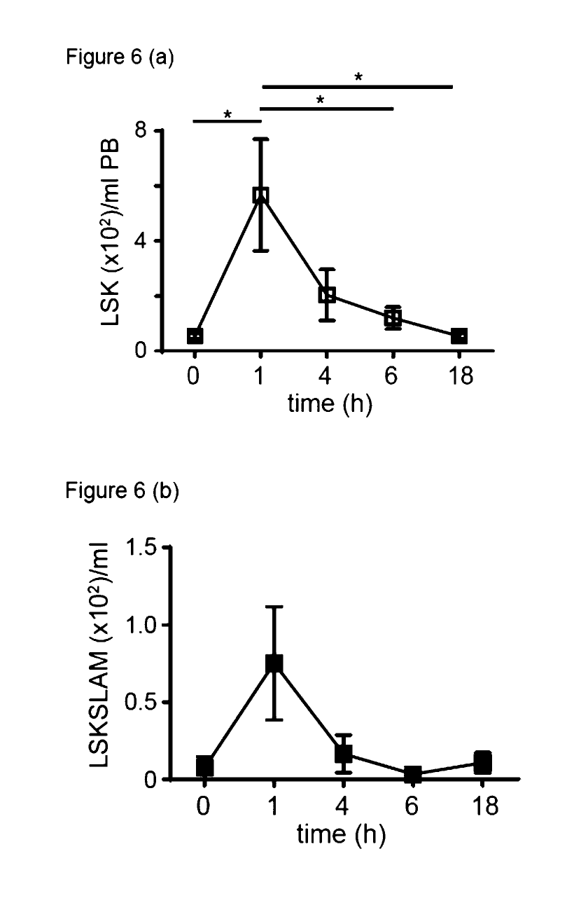

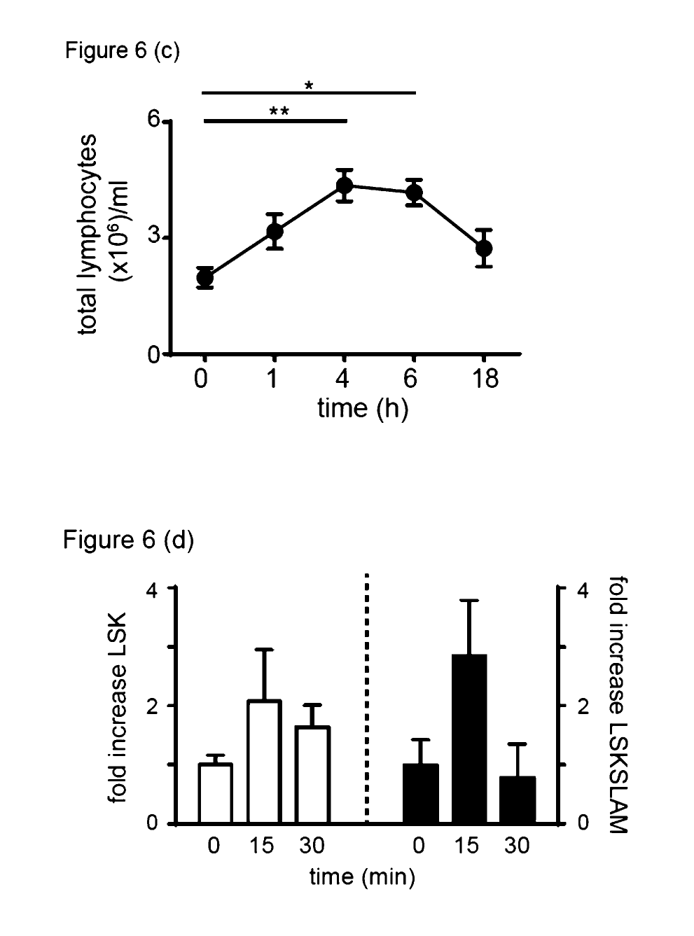

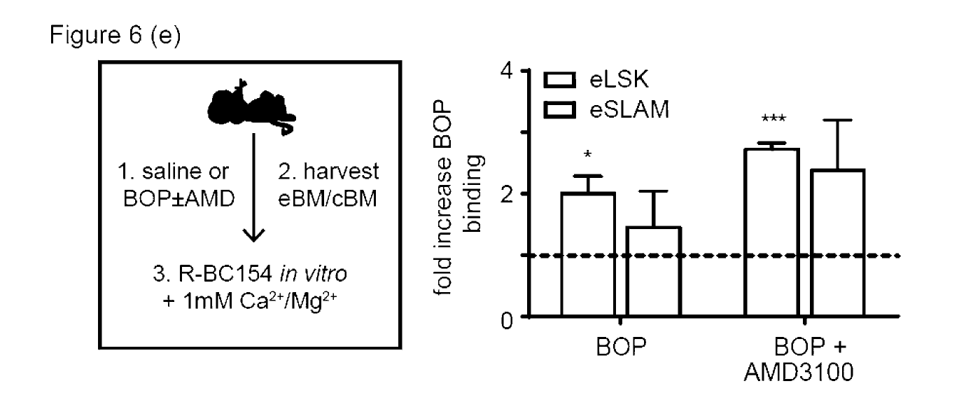

As BOP rapidly and preferentially binds BM HSC, the ability of BOP to mobilize HSC to the peripheral blood (PB) was initially analyzed in a dose and time response assay and by quantifying progenitors (LSK) and HSC (LSKSLAM) post subcutaneous BOP administration (FIG. 5a,b). The administration of BOP resulted in a rapid, significant and dose dependent increase in PB progenitors and HSC (FIG. 5a), which peaked 60 minutes after a single dose (FIG. 5b) and returned to baseline within 4 hours post-administration (FIG. 6a,b). Furthermore, whilst there was an initial significant increase in total PB lymphocytes, these also decreased to baseline by 18 hours (FIG. 6c).

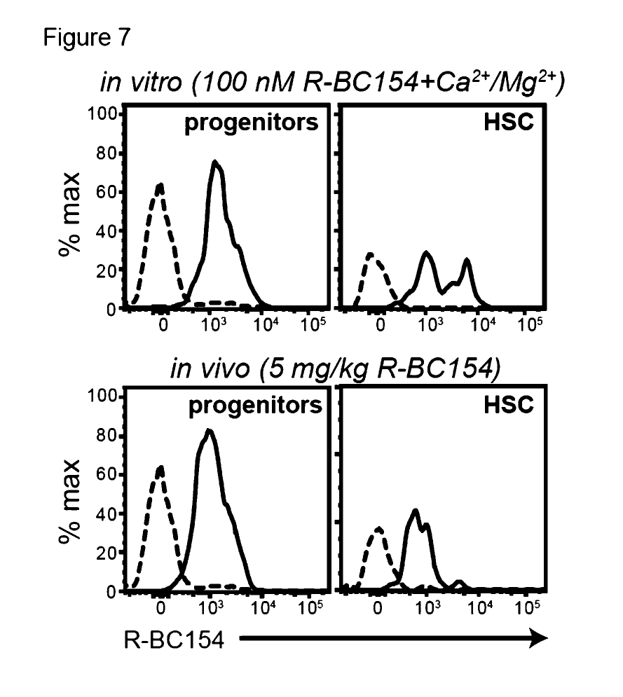

In contrast, R-BC154 (IXb), although capable of efficiently binding BM progenitors (LSK cells) and HSC (LSKSLAM cells) in vitro (FIG. 7), only resulted in moderate increases in PB progenitors and HSC when administered in vivo (FIG. 6d). The reduced in vivo efficacy of R-BC154 (IXb) relative to BOP is most likely the result of the former having lower binding affinities as determined by association and dissociation kinetics studies in vitro and thus reduced inhibitory potency towards integrin-dependent adhesive interactions.

### Example 7: Enhanced HSC Mobilization Using BOP in Combination with AMD310

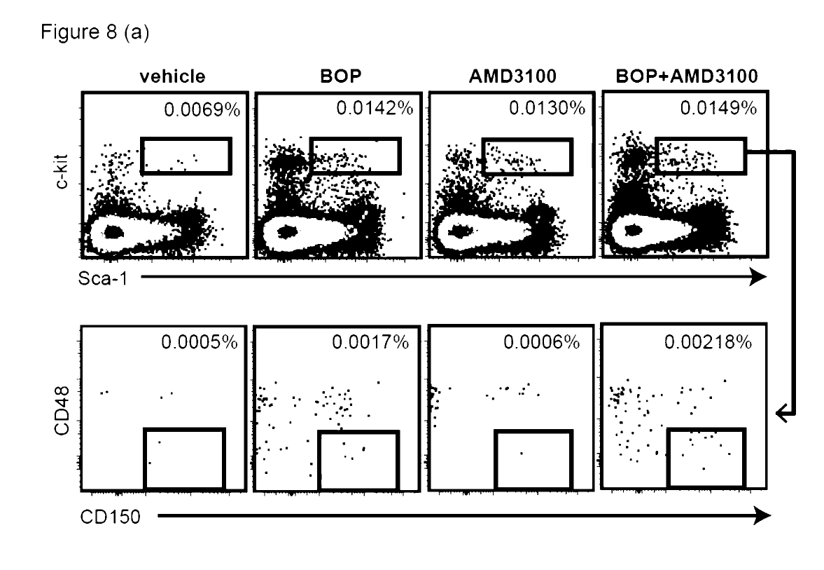

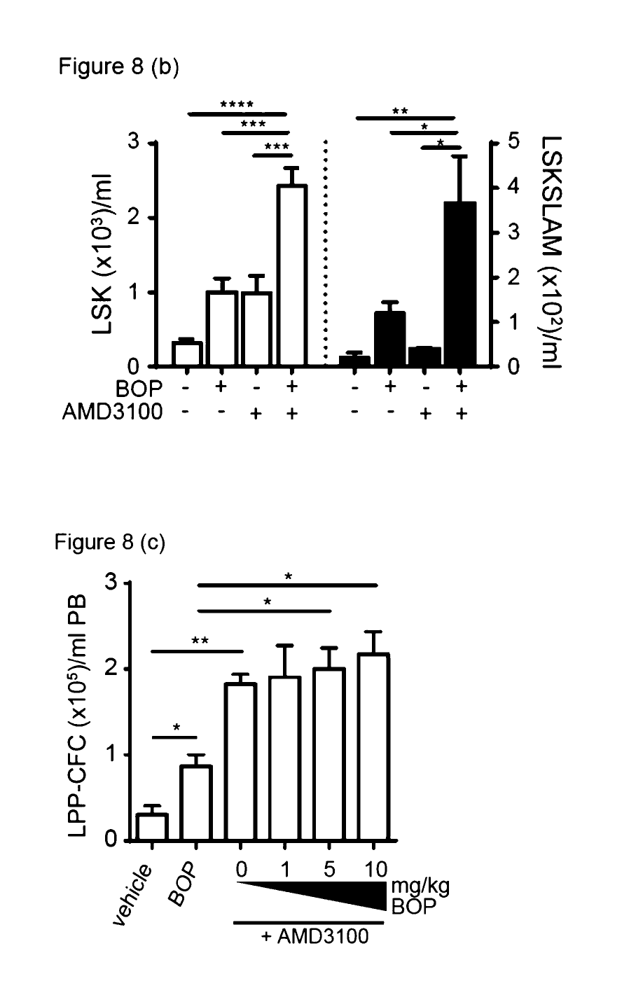

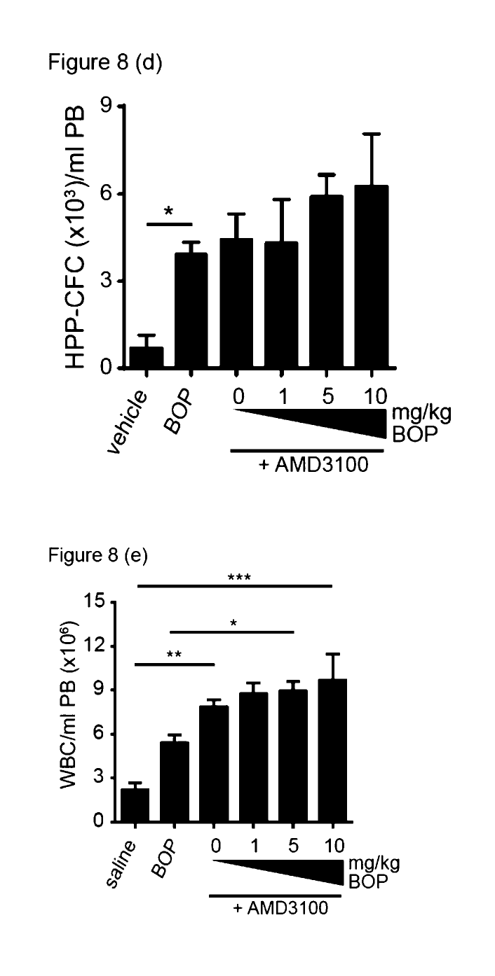

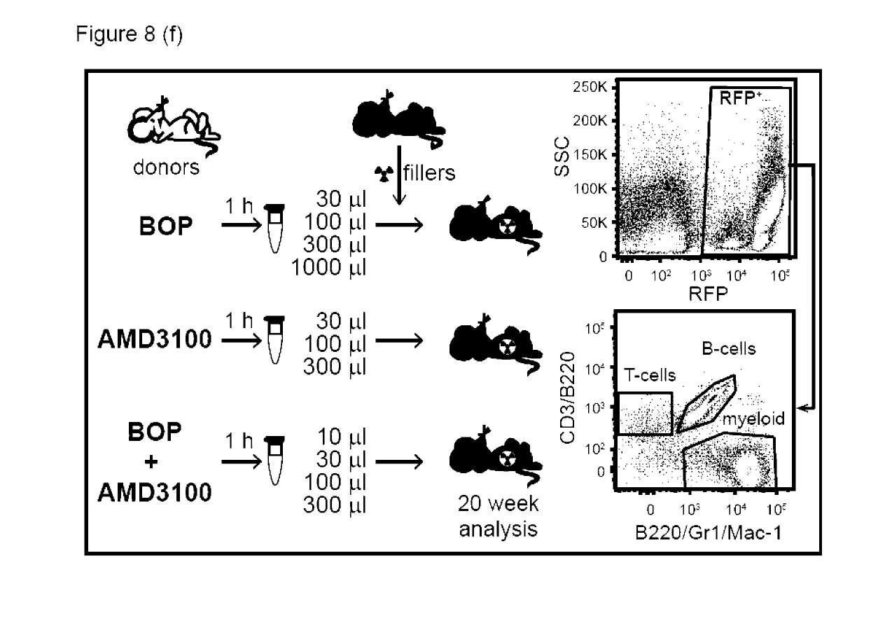

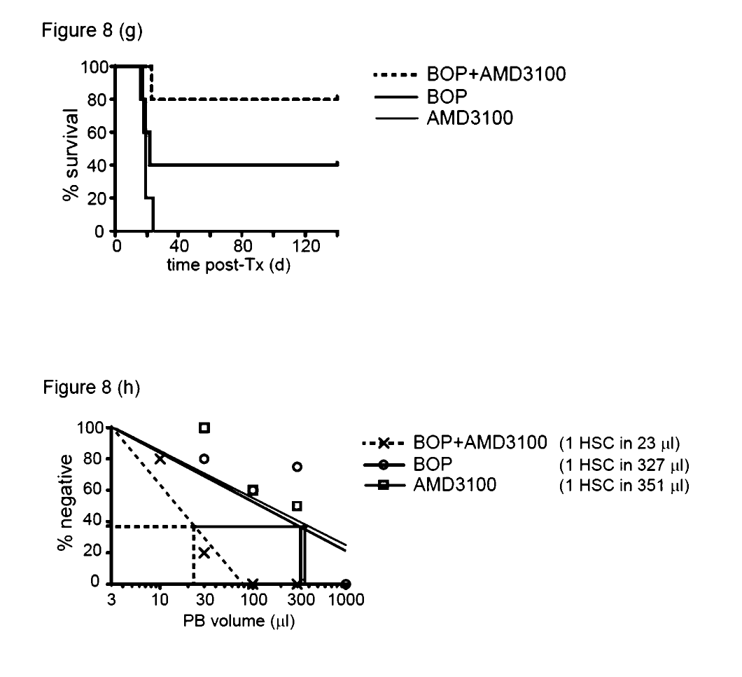

Co-administration of AMD3100 with BOP did not give a significant increase in the proportion of LSK cells in PB compared to mice treated with AMD3100 alone (FIG. 8a). However, the addition of BOP with AMD3100 was found to give a significant increase in the proportion of LSKSLAM cells within PB when compared to BOP or AMD3100 alone (p<0.05). Consequently, the combination of BOP and AMD3100 did not mobilize greater numbers of LSK cells in PB (FIG. 8b) relative to groups receiving BOP or AMD3100 alone, but was found to give significant increases in the number of PB LSKSLAM cells (FIG. 8b).

To confirm whether the phenotypic characterization of LSK and LSKSLAM cells in mobilized PB is reflective of functional HSC and progenitors, PB was assessed for the presence of low-proliferative potential (LPP) and high-proliferative potential (HPP) colony forming cells (CFC). LPP-CFC are representative of committed progenitors and HPP-CFC has been shown to correlate strongly with cells that possess repopulation potential in vivo. PB mobilized by either BOP or AMD3100 exhibited greater LPP-CFC (FIG. 8c) and HPP-CFC (FIG. 8d) content relative to the saline control. However, the combination of BOP and AMD3100 did not mobilize PB with significantly greater LPP-CFC (FIG. 8c) or HPP-CFC (FIG. 8c) when compared to BOP and AMD3100 alone, despite superior mobilization of immunophenotypic LSKSLAM. These results are reflective of the moderate correlation between the detected LSK and LSKSLAM content and the observed HPP-CFC (r²=0.47 and 0.46) and LPP-CFC content in mobilized blood (r²=0.54 and 0.36), respectively (FIG. 8e).

To determine whether BOP mobilized PB comprised true HSC with long-term multi-lineage engraftment potential, limiting dilution transplant analysis was performed. Limiting volumes of PB from RFP donors mobilized by BOP, AMD3100 or the combined treatment of BOP and AMD3100 were transplanted into lethally irradiated C57 recipients and multi-lineage reconstitution assessed up to 20 weeks (FIG. 8f). Significantly greater survival was observed in recipients receiving PB mobilized by the BOP and AMD3100 combination compared to the AMD3100 alone group (FIG. 8g). Poisson regression analysis using L-calc revealed PB mobilized by the BOP and AMD3100 combination gave a greater repopulation frequency of 1 in 23 μl PB (95% Cl=1 in 10 to 1 in 51) compared to BOP (1 in 322; 95% Cl=1 in 127 to 1 in 612) and AMD3100 (1 in 351; 95% Cl=1 in 128 to 1 in 958) alone, conferring greater than 10-fold improvements to the mono-therapy approach (p<0.005) (FIG. 8h). The long-term transplant outcomes was most accurately predicted by the LSKSLAM content in mobilized PB relative to determination of LPP-CFC, HPP-CFC and LSK. These results indicate that treatment with BOP, either alone or in combination with AMD3100, mobilizes long-term repopulating HSC and validates flow cytometric monitoring of the LSKSLAM phenotype as a fast, convenient and moderately “real time” assay for HSC mobilization.

### Example 8: Enhanced HSC Mobilization Using BOP in Combination with Targeting CXCR4

Administration of AMD3100 resulted in similar increases in WBC counts (7.8±1.5×10⁶/ml) relative to BOP (7.0±0.8×10⁶/ml) and comparable increases in the proportion of progenitors (LSK cells) in the PB (FIG. 8a). However, BOP resulted in a significantly greater increase in the proportion of HSC (LSKSLAM cells) in the PB relative to AMD3100 (FIG. 8b: p<0.05), suggesting AMD3100 predominantly mobilizes progenitors, while BOP also mobilizes HSC. In addition, the combination of BOP and AMD3100 synergistically mobilized WBC (16.4±1.9×10⁶/ml), progenitors and HSC cells compared to BOP or AMD3100 alone (FIG. 8b). Interestingly, significantly greater binding of BOP to endosteal LSK was observed relative to their central counterparts in vivo (FIG. 6e).

### Example 9: Inhibition of α₉β₁ in Combination with α₄β₁ and CXCR4 Enhances HSC Mobilization Compared to Specifically Targeting α₄β₁ Alone or α₄β₁ in Combination with CXCR4

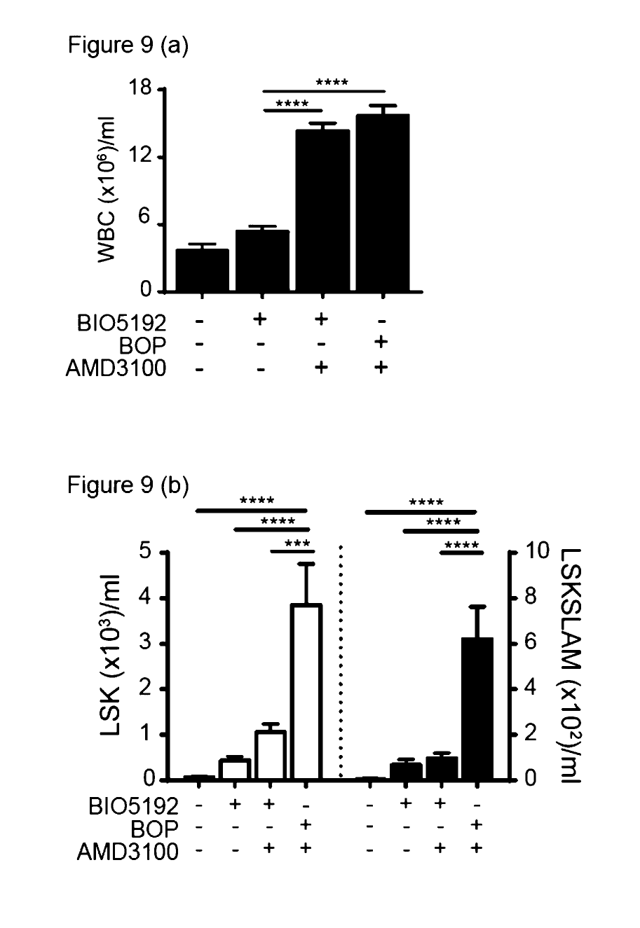

To determine whether co-inhibition of α₉β₁ provides a mobilization advantage over the inhibition of α₄β₁ alone or in combination with CXCR4, the selective α₄β₁ inhibitor BIO5192 was used. BIO5192 has been reported to mobilize CFUs and long-term repopulating HSC with and without AMD3100 but the specific cell types mobilized were not investigated. Intravenous administration of BIO5192 resulted in only moderate increases in WBC counts (FIG. 9a), progenitors (LSK cells) and HSC (LSKSLAM cells) in the PB (FIG. 9b). Co-administration of BIO5192 with AMD3100 produced significant increases in total WBC (FIG. 9a) but only a moderate 2.4-fold increase in progenitors and 1.4-fold increase in HSC compared to BIO5192 alone (FIG. 9b). This is in stark contrast to the combination of BOP and AMD3100, which mobilized significantly greater numbers of progenitors and HSC (FIG. 9b) despite inducing similar PB WBC counts (FIG. 9a). These data demonstrate that concomitant binding to CXCR4 and α₄β₁ results in mobilization of predominantly committed WBC but in contrast, co-binding to α₄β₁ and α₉β₁ results in significantly increased HSC mobilization.

### Example 10: Inhibition of Asp and α₄β₁ with and without CXCR4 Mobilizes Functional HSC

To confirm whether the LSKSLAM and LSK cell phenotype in mobilized PB was reflective of functional HSC and progenitors with long-term multi-lineage engraftment potential, limiting dilution transplant analysis was performed using BOP, AMD3100 or the combination thereof. Greater survival was observed in recipients receiving 30 μl PB mobilized using the combination of BOP and AMD3100 compared to both BOP and AMD3100 alone (p<0.05) (FIG. 8a). Furthermore, Poisson regression analysis following limiting dilution transplant revealed PB mobilized by the BOP and AMD3100 combination resulted in a greater repopulation frequency of 1 HSC in 23 μl PB (95% Cl=1 HSC in 10 μl to 1 HSC in 51 μl) compared to BOP (1 HSC in 327 μl; 95% Cl=1 HSC in 150 μl to 1 HSC in 715 μl) or AMD3100 (1 HSC in 351 μl; 95% Cl=1 HSC in 128 μl to 1 HSC in 958 μl) alone, highlighting a greater than 10-fold improvement to mono-therapy (p<0.005) (FIG. 8h). These results demonstrate that treatment with BOP either alone or in combination with AMD3100, mobilizes long-term repopulating HSC and validates flow cytometric monitoring of the LSKSLAM phenotype as a rapid method for assessing murine HSC mobilization.

### Example 11: The Combination of BOP and AMD3100 is an Effective and Rapid Alternative to G-CSF Mobilization

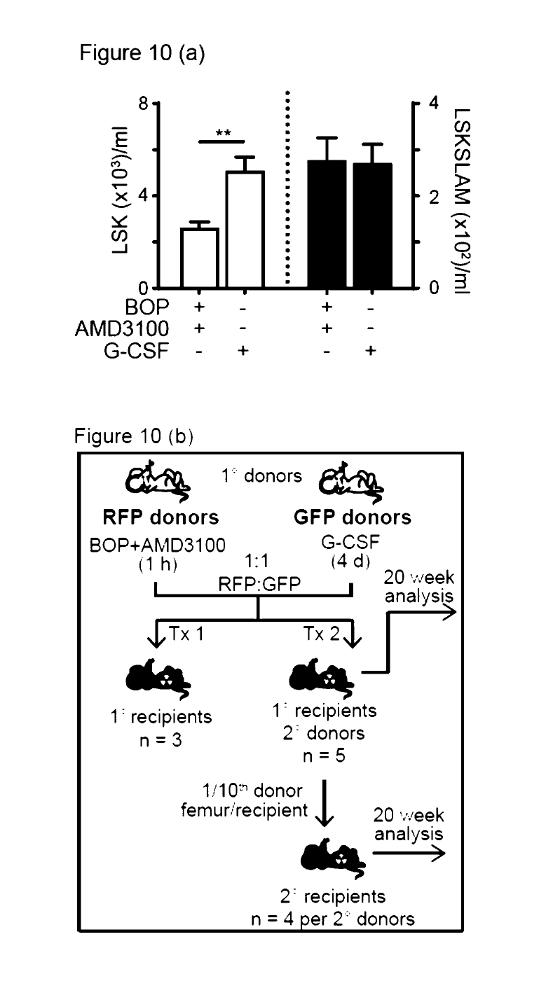

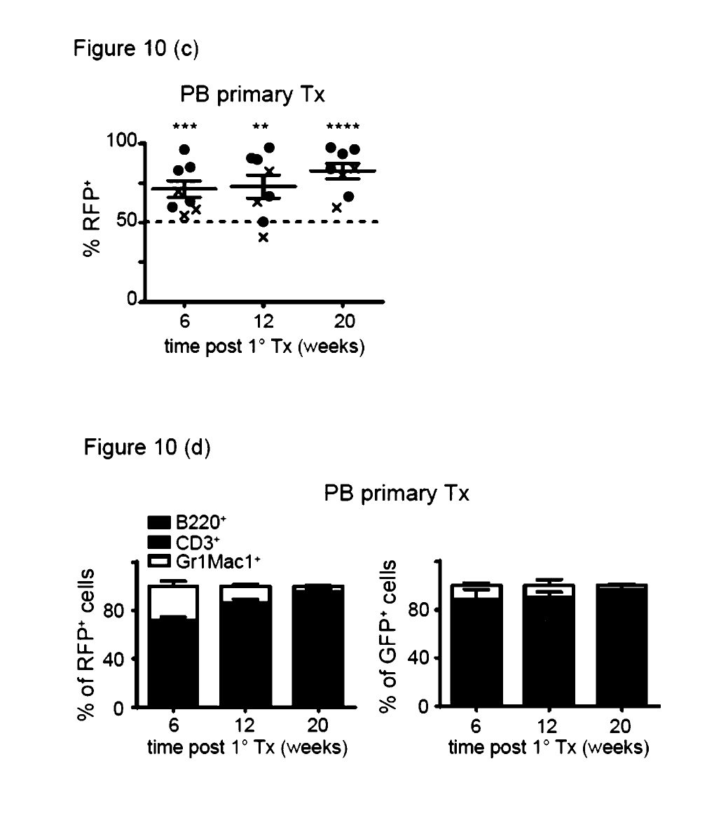

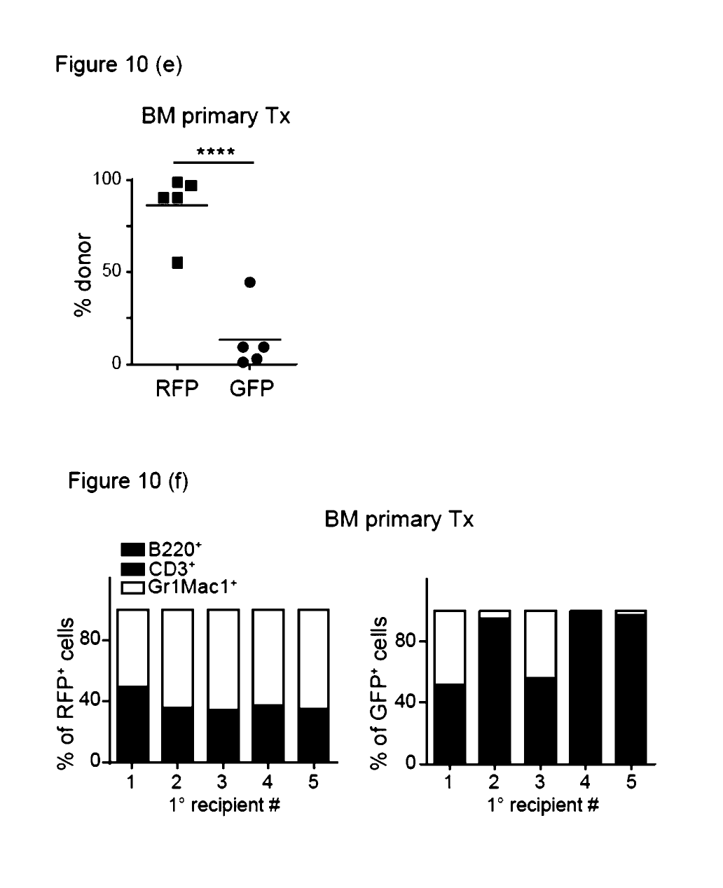

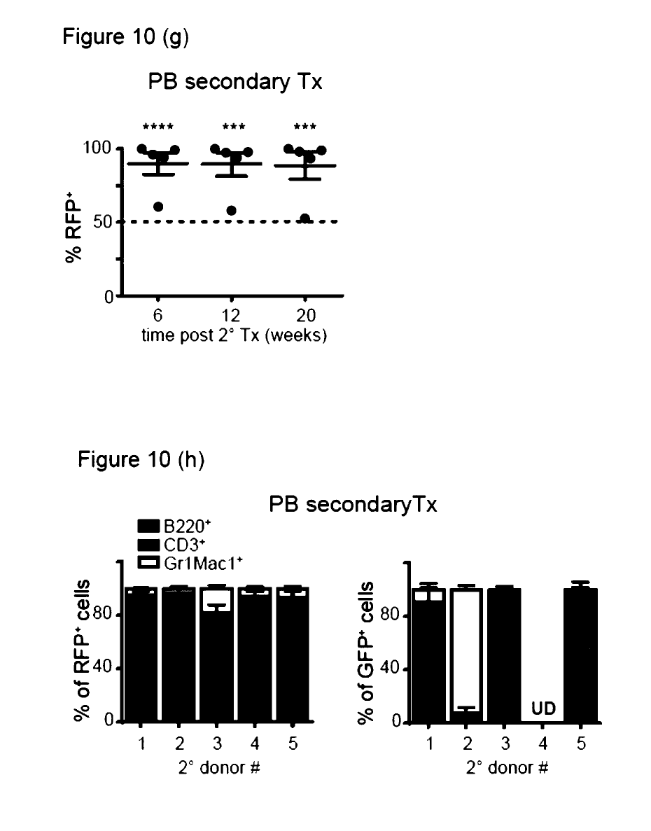

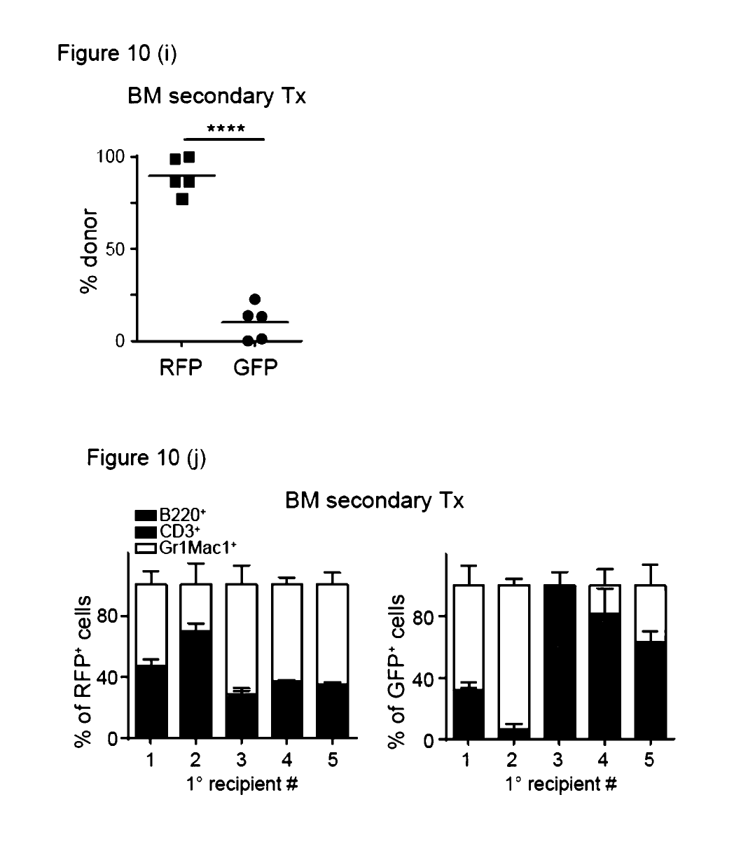

In comparison to mobilization with 4-days of G-CSF, equivalent numbers of HSC were mobilized using a single dose of the combination of BOP and AMD3100 (FIG. 10a). However, interestingly a significantly greater number of progenitors were mobilized using G-CSF alone (FIG. 10a). To compare the hematopoietic potential of PB mobilized with multiple doses of G-CSF versus a single dose of BOP and AMD3100, a competitive long-term reconstitution assay was used (FIG. 10b). Despite equivalent numbers of HSC being mobilized following G-CSF and the combination of BOP and AMD3100 (FIG. 10a), significantly enhanced short and long-term multi-lineage engraftment was observed using the latter (FIG. 10d and FIG. 10 e,f). Furthermore, this significantly greater engraftment than was mathematically expected was maintained in secondary transplants (FIG. 10 g,h and FIG. 10 i,j). The greater engraftment observed with BOP and AMD3100 mobilized PB suggests cells with the LSK/LSKSLAM phenotype mobilized by G-CSF have reduced hematopoietic potential, which is consistent with previous findings demonstrating G-CSF mobilized LSK cells have significantly impaired engraftment potential relative to native BM LSK.

### Example 12: The Combination of BOP and AMD3100 Also Effectively Mobilizes Human CD34⁺ Stem and Progenitor Cells

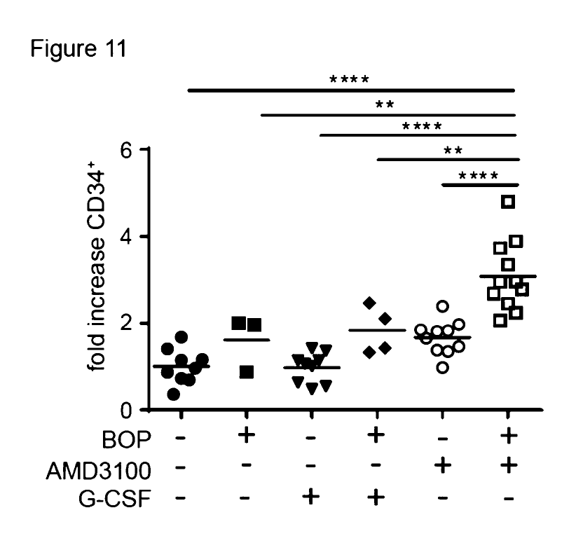

To determine whether HSC mobilization using BOP is equivalent in humans, huNSG mice were utilized. Treatment with a single dose of BOP or AMD3100, or multiple doses of G-CSF for 4-days alone did not result in a significant increase in PB human WBC or human CD34 stem and progenitors (FIG. 11). In contrast, a single dose of BOP in combination with AMD3100 resulted in a significant increase in both human WBC and stem and progenitors (FIG. 11). These data demonstrate huNSG mice are a useful surrogate model for human HSC mobilization and demonstrate the promising efficacy of BOP and AMD3100 for rapid clinical mobilization of human CD34⁺ cells.

### Example 13: Preparation of N-(3-pyridinesulfonyl)-L-prolyl-L-O-(1-pyrrolidinyl carbonyl)tyrosine (Py-BOP)

Py-BOP, which differs from BOP by having a pyridine ring instead of a benzene ring was also synthesised, as shown in the following Scheme 3:

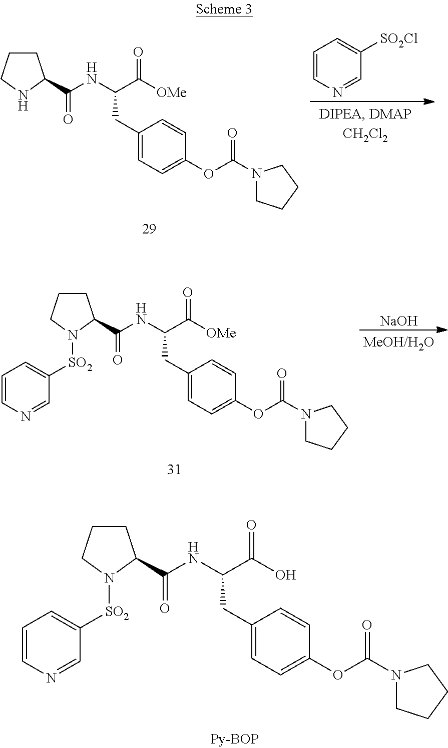 [SMILES: COC(=O)[C@H](Cc1ccc(OC(=O)N2CCCC2)cc1)NC(=O)[C@@H]1CCCN1 ; O=S(=O)(Cl)c1cccnc1 ; COC(=O)[C@H](Cc1ccc(OC(=O)N2CCCC2)cc1)NC(=O)[C@@H]1CCCN1S(=O)(=O)c1cccnc1 ; "NaOH" ; O=C(O)[C@H](Cc1ccc(OC(=O)N2CCCC2)cc1)NC(=O)[C@@H]1CCCN1S(=O)(=O)c1cccnc1]

Thus, amine 29 was reacted with 3-pyridinesulfonylchloride to give sulfonamide 31, which was subsequently hydrolysed with sodium hydroxide to give Py-BOP in excellent overall yield.

By way of exemplification the actual reaction conditions for the formation of Py-BOP, starting from amine 29 is provided herein.

### Step 1: N-(3-pyridinesulfonyl)-L-prolyl-L-O-(1-pyrrolidinycarbonyl)tyrosine methyl ester (31)

Diisopropyl ethyl amine (DIPEA) (110 μl, 0.63 mmol) was added to a stirred solution of amine 29 (98 mg, 0.252 mmol), 3-pyridinesulfonylchloride (295 μl, 0.33 mmol; 200 mg/ml in CH₂Cl₂) and 4-dimethylaminopyridine (DMAP) (5 mg, 0.041 mmol) in CH₂Cl₂ (5 ml). The mixture was stirred for 2 h at room temperature under N₂, washed with sat. aq. NaHCO₃, brine, dried (MgSO₄) and concentrated under reduced pressure. The residue was purified by flash chromatography (5% to 10% MeOH/EtOAc) to give the product 31 (129 mg, 96%) as a colourless oil. δ_{H} (400 MHz, CDCl₃) 1.51-1.62 (3H, m), 1.89-2.00 (4H, m), 2.06-2.09 (1H, m), 3.05 (1H, dd, J=7.5, 14.0 Hz), 3.16 (1H, m), 3.27 (1H, dd, J=5.6, 14.0 Hz), 3.41 (1H, m), 3.46 (2H, t, J=6.6 Hz), 3.55 (2H, t, J=6.8 Hz), 3.78 (3H, s), 4.11 (1H, dd, J=2.8, 8.5 Hz), 4.84 (1H, dt, J=5.7, 11.4 Hz), 7.06-7.14 (5H, m), 7.50 (1H, ddd, J=1.0, 5.0, 8.2 Hz), 8.13 (1H, ddd, J=1.7, 2.5, 8.1 Hz), 8.85 (1H, dd, J=1.6, 4.8 Hz), 9.07 (1H, dd, J=0.8, 2.5 Hz).

### Step 2: N-(3-pyridinesulfonyl)-L-prolyl-L-O-(1-pyrrolidinylcarbonyl)tyrosine (Py-BOP)

0.2 M NaOH (1.3 ml, 0.261 mmol) was added to a solution of ester 31 (116 mg, 0.218 mmol) in EtOH (5 ml) and the mixture stirred for 2 h at room temperature, concentrated and then purified by C₁₈ reverse phase chromatography (30%-50% MeOH/H₂O) to give the product (99 mg, 88%) as a colourless glass. δ_{H} (400 MHz, D₂O) 1.55-1.69 (2H, m), 1.75-1.92 (6H, m), 3.01 (1H, dd, J=8.1, 14.0 Hz) 3.21-3.27 (2H, m), 3.32-3.36 (2H, m), 3.40-3.48 (3H, m), 4.12 (1H, dd, J=4.4, 8.7 Hz), 4.46 (1H, dd, J=4.9, 8.0 Hz), 7.03 (2H, d, J=8.5 Hz), 7.28 (2H, d, J=8.5), 7.64 (1H, dd, J=5.0, 8.2 Hz), 8.15 (1H, ddd, J=1.5, 2.2, 8.0 Hz), 8.78 (1H, dd, J=1.2, 4.9 Hz), 8.89 (1H, d, J=1.7 Hz); δ_{c} (100 MHz, D₂O) 24.22, 24.64, 25.26, 30.86, 37.01, 46.54, 46.61, 49.67, 56.10, 62.12, 121.86, 125.13, 130.59, 132.83, 135.15, 136.57, 147.24, 149.69, 153.55, 155.17, 172.85, 177.38; HRMS (ESI⁺) m/z 539.1574 (C₂₄H₂₈NaN₄O₇S [M+Na]⁺ requires 539.1571).

### Example 14: The Combination of Py-BOP and AMD3100 Effectively Mobilizes Human LSK and LSKSLAM Cells

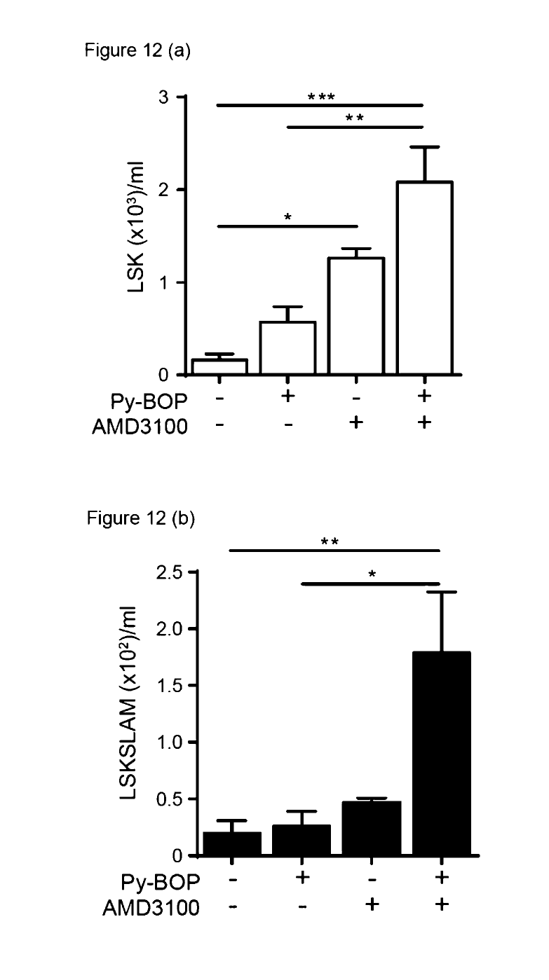

The administration of Py-BOP in combination with AMD3100 in vivo induced a significant increase in PB progenitors (FIG. 12a) and HSC (FIG. 12b) after 1 hour, demonstrating rapid and effective mobilisation.

### Example 15: BOP Rapidly Mobilises HSC and BCP-ALL, with α₉β₁ Playing a Critical Role

The administration of BOP in vivo induced a dose dependent increase in PB HSC (FIG. 5a), which peaked at 1 hour, demonstrating rapid and effective mobilisation (FIG. 5b). Furthermore, the addition of AMD3100 to BOP resulted in a significant and synergistic increase in the number of mobilised HSC (FIG. 8b) and these had a significantly higher frequency of cells with long-term multi-lineage engraftment potential (FIG. 8h).

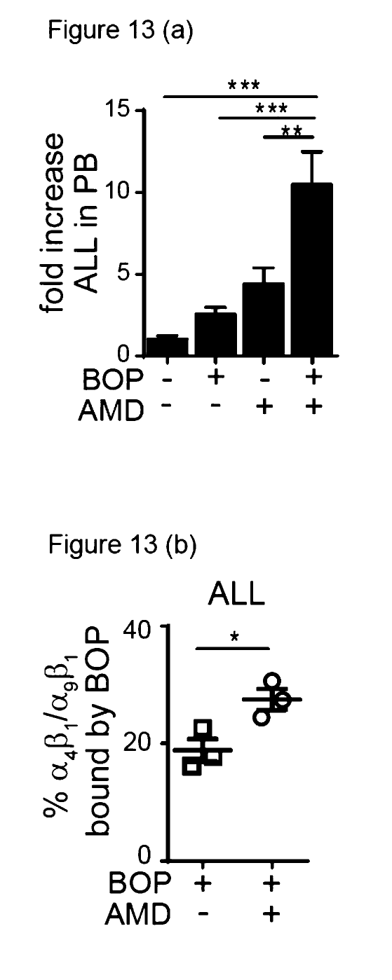

Importantly, while similar increases in WBC counts were observed when either the α4β1 specific inhibitor BIO519239 or the dual α4β1/α9β1 inhibitor BOP was used in combination with AMD3100 significantly greater HSC mobilisation was observed following the latter (FIG. 9a) highlighting α₉β₁ as the critical receptor for the preferential egress of HSC. The use of BOP in combination with AMD3100 also rapidly and effectively mobilised human CD34+(shown above) and BCP-ALL (FIG. 13a) cells and this was significantly increased compared to BOP or AMD3100 alone. Importantly, significantly greater binding of BOP was observed to endosteally located ALL cells when BOP was used in combination with AMD3100 relative to BOP alone, demonstrating AMD3100 enhances α₄β₁/α₉β₁ activity in this BM region in vivo (FIG. 13b). These data demonstrate that BOP plus AMD3100 effectively mobilise human CD34+ and BCP-ALL cells in huNSG xenograft models and highlight the efficacy of BOP plus AMD3100 for both clinical mobilisation and dislodgement of BM ALL cells.

The above examples show that inhibition of α₉β₁/α₄β₁ integrins using a small molecule antagonist BOP induces the rapid mobilization of long-term repopulating HSC through inhibition of integrin-dependent binding to VCAM-1 and Opn.

Using a fluorescent small molecule integrin antagonist (R-BC154 (IXb)) that binds to α₄β₁ and α₉β₁ integrins only when activated by divalent metal cations, it is shown for the first time that the activation state of these two β₁ integrins on murine and human HSC are intrinsically activated and differentially specified by the endosteal niche in vivo.

These examples show that BM cells within the endosteal niche including HSC and progenitor cells express α₉β₁/α₄β₁ integrins that are in a higher affinity binding state beyond what is observed within the central medullary compartment.

Applicants show that determination of LSKCD150⁺CD48⁻ (LSKSLAM) can be used as a rapid and convenient surrogate screen for mobilization of putative HSC, which reflected long-term transplant outcomes more closely than quantifying CFC content of mobilized blood. Specifically, it was found that BOP, either alone or in combination with AMD3100, effectively mobilized phenotypic LSKSLAM in a manner that correlated better to LT-HSC than determination of HPP-CFC, LPP-CFC or LSK cell content. Thus, assessment of HSC mobilization based solely on short-term colony forming assays would have otherwise discounted the combination of BOP and AMD3100 as an effective mobilization approach.

In the current study G-CSF failed to mobilize CD34⁺ haematopoietic stem and progenitors in humanised NODSCIDIL2Rγ^{-/-} mice. In contrast, the combination of BOP and AMD3100 gave significant mobilization of human CD34⁺ cells and suggests this strategy may be applicable to clinical mobilization of human patients and donors.

Applicants have demonstrated that targeting α₉β₁/α₄β₁ using a single dose of BOP, induces rapid mobilization of long-term repopulating HSC through inhibiting integrin-dependent binding, most likely to trOpn and VCAM-1. Using the fluorescent BOP analogue, R-BC154 (IXb), it is shown that a significant proportion of binding to human and murine HSC occurs through α₉β₁, whereas binding to lineage committed cells is almost exclusively through α₄β₁. Consequently, inhibition of α₉β₁ was found to provide an added advantage to HSC mobilization yields over inhibition of α₄β₁ alone as demonstrated by the significantly greater mobilization of HSC and progenitors using BOP in comparison to the selective α₄β₁ antagonist BIO5192; particularly when used in combination with AMD3100. The reduced synergism in HSC mobilization, but not WBC mobilization using BIO5192 in combination with AMD3100 compared to BOP in combination with AMD3100 supports targeting α₄ biases towards progenitor mobilization whereas targeting α₉ preferentially mobilizes HSC. The pronounced increase in AMD3100-mediated mobilization using BOP relative to BIO5192 suggests the role of integrin α₉β₁ in HSC mobilization is magnified with concomitant targeting of CXCR4.

In summary, it is now shown that a single dose of BOP, a small molecule targeting α₄β₁ and α₉β₁ integrins, effectively and rapidly mobilizes HSC with long-term multi-lineage engraftment potential, identifying a previously unrecognized role for α₉β₁ in HSC mobilization. When used in combination with CXCR4 inhibitors such as AMD3100, significantly enhanced mobilization of long-term repopulating HSC was observed relative to G-CSF. The efficacy of HSC mobilization using the BOP and AMD3100 combination was recapitulated in the mobilization of CD34⁴⁺ cells in humanised NODSCIDIL2Rγ^{-/-} mice. Using the related fluorescently labelled integrin antagonist R-BC154 (IXb), it is shown that this class of compounds bind murine and human HSC and progenitors via endogenously activated/primed α₄β₁ and α₉β₁ within the endosteal niche. Furthermore, it is shown that the majority of R-BC154 (IXb)/BOP binding to human HSC occurs via α₉β₁, which is absent on lineage committed cells. These results highlight an effective and convenient strategy of therapeutically targeting endosteal HSC which address many of the shortcomings associated with G-CSF and paves the way for the development of additional selective small molecule α₉β₁ integrin antagonists for preferential HSC mobilization.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

### REFERENCES

1. J. Grassinger, D. N. Haylock, M. J. Storan, G. O. Haines, B. Williams, G. A. Whitty, A. R. Vinson, C. L. Be, S. H. Li, E. S. Sorensen, P. P. L. Tam, D. T. Denhardt, D. Sheppard, P. F. Choong and S. K. Nilsson, Blood, 2009, 114, 49-59.
2. D. N. Haylock, B. Williams, H. M. Johnston, M. C. P. Liu, K. E. Rutherford, G. A. Whitty, P. J. Simmons, I. Bertoncello and S. K. Nilsson, Stem Cells, 2007, 25, 1062-1069.
3. J. Grassinger, B. Williams, G. H. Olsen, D. N. Haylock and S. K. Nilsson, Cytokine, 2012, 58, 218-225.
4. Nilsson, S. K. et al. Osteopontin, a key component of the haematopoietic stem cell niche and regulator of primitive haematopoietic progenitor cells. Blood 106, 1232-1239, doi: 10.1182/blood-2004-11-4422 (2005).
5. Grassinger, J. et al. Thrombin-cleaved osteopontin regulates haematopoietic stem and progenitor cell functions through interactions with alpha(9)beta(1) and alpha(4)beta(1) integrins. Blood 114, 49-59, doi:DOI 10.1182/blood-2009-01-197988 (2009).
6. Bartelmez, S. H. et al. Interleukin-1 Plus Interleukin-3 Plus Colony-Stimulating Factor-I Are Essential for Clonal Proliferation of Primitive Myeloid Bone-Marrow Cells. Experimental hematology 17, 240-245 (1989).
7. Herbert, K. E., Levesque, J. P., Haylock, D. N. & Princes, M. The use of experimental murine models to assess novel agents of haematopoietic stem and progenitor cell mobilization. Biol Blood Marrow Tr 14, 603-621, doi:DOI 10.1016/j.bbmt.2008.02.003 (2008).
8. Cao, B. et al. Design, synthesis and binding properties of a fluorescent alpha(9)beta(1)/alpha(4)beta(1) integrin antagonist and its application as an in vivo probe for bone marrow haemopoietic stem cells. Org. Biomol. Chem. 12, 965-978, doi: Doi 10.1039/C3ob42332h (2014).
9. Pepinsky, R. B. et al. Comparative assessment of the ligand and metal ion binding properties of integrins alpha 9 beta 1 and alpha 4 beta 1. Biochemistry 41, 7125-7141, doi: Doi 10.1021/Bi020024d (2002).
10. Schofield, R. Relationship between Spleen Colony-Forming Cell and Haematopoietic Stem-Cell—Hypothesis. Blood Cells 4, 7-25 (1978)
11. Shultz, L. D., Brehm, M. A., Garcia-Martinez, J. V. & Greiner, D. L. Humanized mice for immune system investigation: progress, promise and challenges. Nat Rev Immunol 12, 786-798, doi: Doi 10.1038/Nri3311 (2012).
12. Ito, R., Takahashi, T., Katano, 1. & Ito, M. Current advances in humanized mouse models. Cell Mol Immunol 9, 208-214, doi:Doi 10.1038/Cmi.2012.2 (2012).
13. Leone, D. R. et a. An assessment of the mechanistic differences between two integrin alpha(4)beta(1) inhibitors, the monoclonal antibody TA-2 and the small molecule BIO5192, in rat experimental autoimmune encephalomyelitis. J. Pharmacol. Exp. Ther. 305, 1150-1162, (2003).

The claims defining the invention are as follows:

1. A method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof and a CXCR4 antagonist or an active portion thereof to the BM stem cell niche.

2. A method according to claim 1 wherein said method further enhances release of HSC and their precursors and progenitors thereof from the BM stem cell niche.

3. A method according to claim 1 or 2 wherein the method further enhances mobilization of the HSC from the BM stem cell niche.

4. A method according to any one of claims 1 to 3 wherein the α₉ integrin is an α₉β₁ integrin or an active portion thereof.

5. A method according to any one of claims 1 to 4 further including administering an antagonist of α₄ integrin or an active portion thereof.

6. A method according to claim 5 wherein the α₄ integrin is an antagonist of α₄β₁ or an active portion thereof.

7. A method according to any one of claims 1 to 6 wherein the antagonist cross-reacts with α₉ and α₄, and optionally cross-reacts with α₉β₁ and α₄β₁.

8. A method according to any one of claims 1 to 7 wherein the antagonist is a compound of Formula (I) or a pharmaceutically acceptable salt thereof having the following formula: 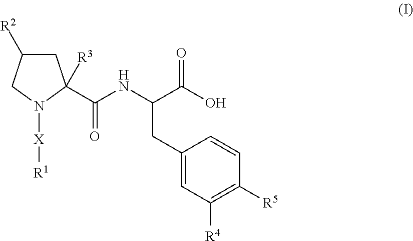
wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁵, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

9. A method according to claim 8 wherein:
R⁴ is H; and
R⁵ is —OR⁷.

10. A method according to claim 8 or claim 9 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

11. A method according to any one of claims 8 to 10 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

12. A method according to any one of claims 8 to 11 wherein the compound of Formula (I) has the following Formula (Ia): 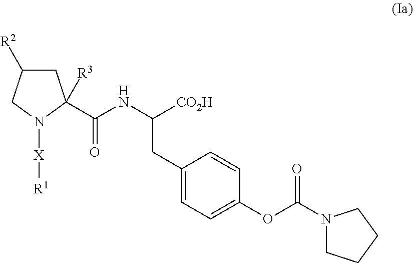
or a pharmaceutically acceptable salt thereof.

13. The method according to any one of claims 8 to 12 wherein R¹ is optionally substituted phenyl.

14. The method according to claim 13, wherein the phenyl is optionally substituted with at least one halogen group.

15. The method according to any one of claims 8 to 13 wherein the compound of Formula (I) has the following Formula (Ib): 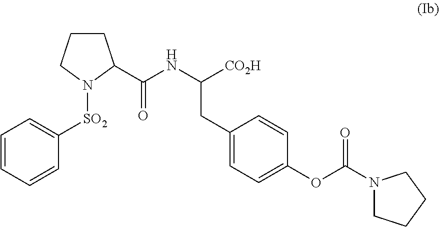 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

16. The method according to claim 15, wherein the compound of formula (I) has the following Formula (Ic): 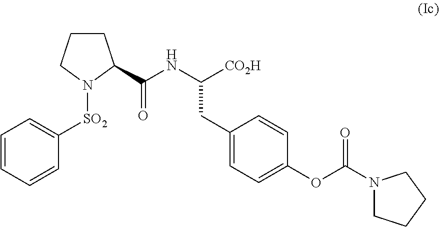 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

17. The method according to any one of claims 8 to 12 wherein R¹ is optionally substituted pyridyl.

18. The method according to claim 17, wherein the compound of Formula (I) has the following Formula (Id): 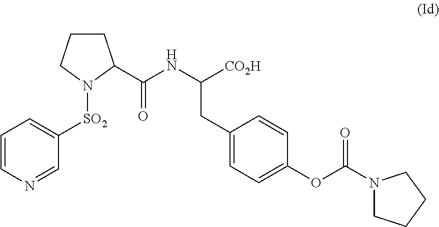 [SMILES: O=C(O)C(Cc1ccc(OC(=O)N2CCCC2)cc1)NC(=O)C1CCCN1S(=O)(=O)c1cccnc1]
or a pharmaceutically acceptable salt thereof.

19. The method according to claim 18, wherein the compound of formula (I) has the following Formula (Ie): 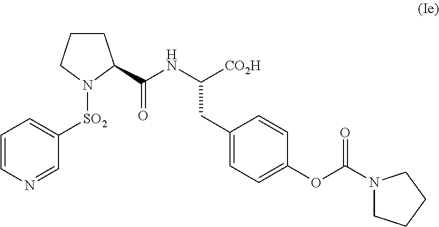 [SMILES: O=C(O)[C@H](Cc1ccc(OC(=O)N2CCCC2)cc1)NC(=O)[C@@H]1CCCN1S(=O)(=O)c1cccnc1]
or a pharmaceutically acceptable salt thereof.

20. A method according to any one of claims 1 to 19 wherein the CXCR4 antagonist or an active portion thereof is selected from the group including bicyclam derivatives, tetrahydroquinoline derivatives, cyclic peptides, para-xylyl-enediamine-based derivatives, isothiourea derivatives and POL6326, POL5551, CCTE-9908 and TG-0054 and optionally, the CXCR4 antagonist is AMD3100.

21. A method according to any one of claims 1 to 20 wherein the antagonist of an α₉ integrin or an active portion thereof and the CXCR4 antagonist or an active portion thereof is administered in combination, simultaneously or sequentially.

22. A method according to any one of claims 1 to 21 wherein the antagonist and the CXCR4 antagonist or an active portion thereof are administered in the absence of G-CSF.

23. A method according to any one of claims 1 to 22 wherein the α₉ integrin antagonist and the CXCR4 antagonist or an active portion thereof are administered intravenously, intradermally, subcutaneously, intramuscularly, transdermally, intraperitoneally or transmucosally; optionally the α₉ integrin antagonist and the CXCR4 antagonist or an active portion thereof are administered intravenously or subcutaneously.

24. A method according to any one of claims 5 to 23 wherein the α₉ integrin antagonist and the CXCR4 antagonist or an active portion thereof are administered simultaneously, consecutively or in combination with an α₄ integrin antagonist.

25. A method according to any one of claims 1 to 24 wherein the HSC and their precursors and progenitors are derived from bone marrow.

26. A method according to claim 25 wherein the HSC and their precursors and progenitors are derived from the stem cell niche, optionally from the endosteal niche.

27. A method according to claim 25 or 26 for enhancing dislodgement of HPC from the BM stem cell binding ligand in vivo or ex vivo.

28. A method according to any one of claims 1 to 27 wherein the HSC and their precursors and progenitors are long term repopulating HSC and their precursors and progenitors and optionally selected from the group including CD34⁺ cells, CD38+, CD90+, CD133+, CD34⁺CD38⁻ cells, lineage-committed CD34⁻ cells, or CD34⁺CD38⁺ cells; optionally CD34⁺ or CD34⁺CD38⁻ cells.

29. A composition for enhancing dislodgement of HSC and their precursors and progenitors thereof from a BM stem cell binding ligand said composition comprising an antagonist of α₉ integrin or an active portion thereof and a CXCR4 antagonist or an active portion thereof.

30. A composition according to claim 29 further enhancing release of HSC and their precursors and progenitors from a BM stem cell binding ligand.

31. A composition according to claim 29 or 30 further enhancing mobilization of HSC from a BM stem cell niche to PB, optionally HPC from a BM stem cell niche to PB.

32. A composition according to any one of claims 29 to 31 wherein the α₉ integrin is an α₉β₁ integrin or an active portion thereof.

33. A composition according to any one of claims 29 to 32 further including an antagonist of α₄ integrin or an active portion thereof.

34. A composition according to claim 33 wherein the α₄ integrin is an antagonist of α₄β₁ or an active portion thereof.

35. A composition according to any one of claims 29 to 34 wherein the antagonist cross-reacts with α₉ and α₄, and optionally cross-reacts with α₉β₁ and α₄β₁.

36. A composition according to any one of claims 29 to 35 wherein the α₉ integrin antagonist is a compound of Formula (I) or a pharmaceutically acceptable salt thereof having the formula: 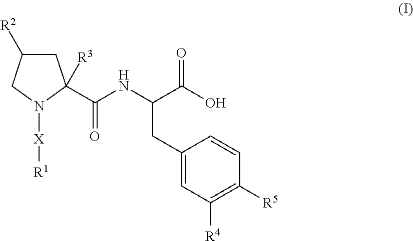
wherein
X is selected from the group consisting of a bond and —SO₂—
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

37. A composition according to claim 36 wherein:
R⁴ is H; and
R⁵ is —OR⁷.

38. A composition according to claim 29 or 37 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

39. A composition according to any one of claims 29 to 38 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

40. A composition according any one of claims 29 to 39 wherein the compound of Formula (I) has the following Formula (Ia): 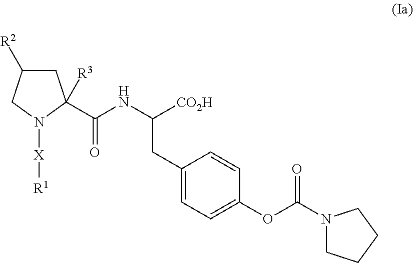
or a pharmaceutically acceptable salt thereof.

41. A composition according to any one of claims 29 to 40 wherein R¹ is optionally substituted phenyl.

42. The composition according to claim 41 wherein the phenyl is optionally substituted with at least one halogen group.

43. The composition according to any one of claims 29 to 42 wherein the compound of Formula (I) has the following Formula (Ib): 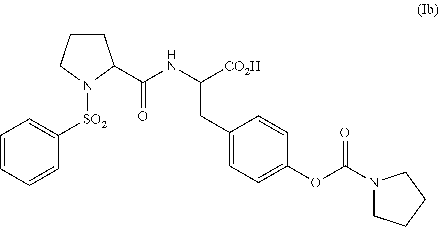 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

44. The composition according to claim 43, wherein the compound of formula (I) has the following Formula (Ic): 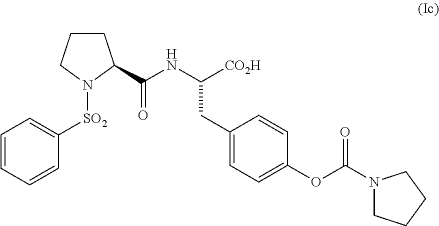 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

45. A composition according to any one of claims 29 to 40 wherein R¹ is optionally substituted pyridyl.

46. The composition according to claim 45 wherein the compound of Formula (I) has the following Formula (Id): 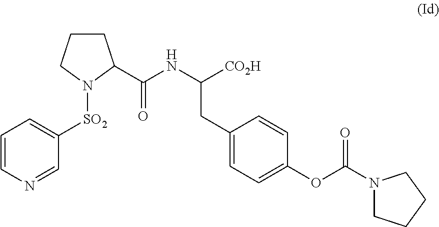 [SMILES: O=C(O)C(Cc1ccc(OC(=O)N2CCCC2)cc1)NC(=O)C1CCCN1S(=O)(=O)c1cccnc1]
or a pharmaceutically acceptable salt thereof.

47. The composition according to claim 46, wherein the compound of formula (I) has the following Formula (Ie): 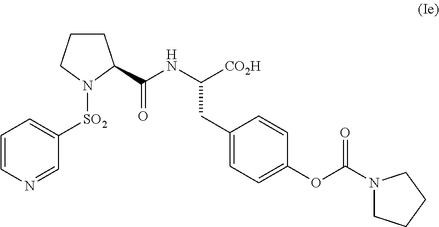 [SMILES: O=C(O)[C@H](Cc1ccc(OC(=O)N2CCCC2)cc1)NC(=O)[C@@H]1CCCN1S(=O)(=O)c1cccnc1]
or a pharmaceutically acceptable salt thereof.

48. A composition according to any one of claims 36 to 47 for administering in the absence of G-CSF.

49. A composition according to any one of claims 36 to 48 for administering intravenously, intradermally, subcutaneously, intramuscularly, transdermally, intraperitoneally or transmucosally; optionally the composition is administered intravenously or subcutaneously.

50. A composition according to any one of claims 36 to 49 wherein the α₉ integrin antagonist and the CXCR4 antagonist or an active portion thereof is administered simultaneously, consecutively or in combination with an α₄ integrin antagonist.

51. A method of harvesting HSC/HPC from a subject said method comprising:
administering an effective amount of an antagonist of α₉ integrin or an active portion thereof and a CXCR4 antagonist or an active portion thereof to a subject wherein said effective amount enhances dislodgement of HSC/HPC and their precursors and progenitors thereof from a BM stem cell binding ligand in a BM stem cell niche;
mobilizing the dislodged HSC to PB; and
harvesting the HSC from the PB.

52. A method according to claim 51 wherein the α₉ integrin antagonist and the CXCR4 antagonist or an active portion thereof is administered in the absence of G-CSF.

53. A method according to claim 51 or 52 wherein the HSC/HPC are further mobilized by the use of other HSC mobilizing agents selected from the group comprising interleukin-17, cyclophosphamide (Cy), Docetaxel and granulocyte-colony stimulating factor (G-CSF).

54. A method according to any one of claims 51 to 53 wherein the effective amount of the integrin antagonist is in the range 25-1000 μg/kg body weight, more preferably 50-500 μg/kg body weight, most preferably 50-250 μg/kg body weight.

55. A method according to any one of claims 51 to 54 wherein the effective amount of the CXCR4 antagonist is in the range 10-1000 ug/kg body weight, more preferably 10-500 ug/kg body weight, most preferably 10-250 ug/kg body weight.

56. A cell composition comprising HSC obtained from a method according to any one of claims 51 to 55.

57. A cell composition comprising HPC obtained from a method according to any one of claims 51 to 55 said composition comprising a greater portion of HPC compared a cell composition mobilized in the absence of an antagonist of an α9 integrin or an active portion thereof and a CXCR4 antagonist or an active portion thereof.

58. A method for the treatment of haematological disorders said method comprising administering a cell composition according to claim 56 or 57.

59. A method for the treatment of haematological disorders in a subject said method comprising administering a therapeutically effective amount of an antagonist of α₉ integrin or an active portion thereof and a CXCR4 antagonist or an active portion thereof to the subject to enhance dislodgement, release or mobilization of HSC from the BM stem cell binding ligand in the BM stem cell niche to the PB.

60. A method according to claim 58 or 59 wherein the α₉ integrin is an α₉β₁ integrin or an active portion thereof.

61. A method according to claim 58 or 59 further including administering an antagonist of α₄ integrin or an active portion thereof.

62. A method according to claim 60 wherein the α₄ integrin is an antagonist of α₄β₁ or an active portion thereof.

63. A method according to any one of claims 59 to 62 wherein the antagonist cross-reacts with α₉ and α₄, and optionally cross-reacts with α₉β₁ and α₄β₁.

64. A method according to any one of claims 59 to 63 wherein the antagonist is a compound of Formula (I) or a pharmaceutically acceptable salt thereof having the formula: 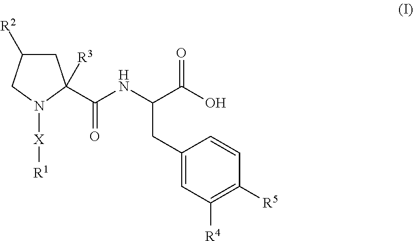
wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

65. A method according to claim 64 wherein:
R⁴ is H; and
R⁵ is —OR⁷.

66. A method according to claim 64 or claim 65 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

67. A method according to any one of claims 64 to 66 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

68. A method according to any one of claims 64 to 67 wherein the compound of Formula (I) has the following Formula (Ia): 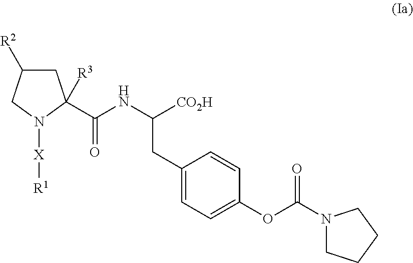
or a pharmaceutically acceptable salt thereof.

69. The method according to any one of claims 64 to 68 wherein R¹ is optionally substituted phenyl.

70. The method according to claim 69, wherein the phenyl is optionally substituted with at least one halogen group.

71. The method according to any one of claims 64 to 70 wherein the compound of Formula (I) has the following Formula (Ib): 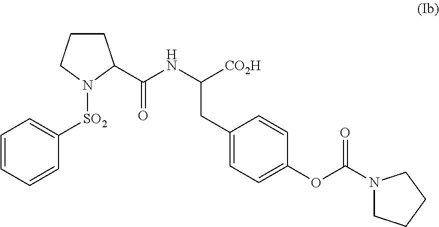 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

72. The method according to claim 71, wherein the compound of formula (I) has the following Formula (Ic): 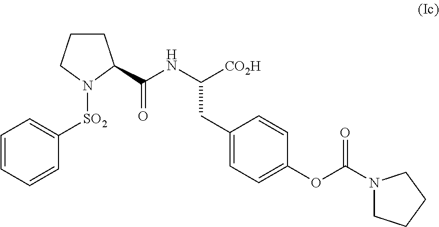 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

73. The method according to any one of claims 64 to 68 wherein R¹ is optionally substituted pyridyl.

74. The method according to claim 73 wherein the compound of Formula (I) has the following Formula (Id): 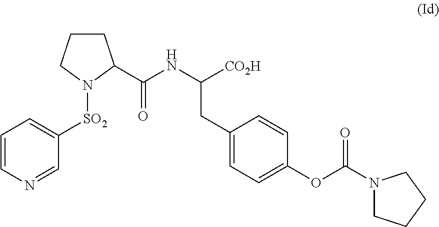 [SMILES: O=C(O)C(Cc1ccc(OC(=O)N2CCCC2)cc1)NC(=O)C1CCCN1S(=O)(=O)c1cccnc1]
or a pharmaceutically acceptable salt thereof.

75. The method according to claim 74, wherein the compound of formula (I) has the following Formula (Ie): 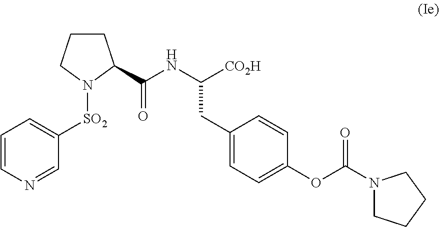 [SMILES: O=C(O)[C@H](Cc1ccc(OC(=O)N2CCCC2)cc1)NC(=O)[C@@H]1CCCN1S(=O)(=O)c1cccnc1]
or a pharmaceutically acceptable salt thereof.

76. A method according to any one of claims 59 to 75 wherein the CXCR4 antagonist or an active portion thereof is selected from the group including bicyclam derivatives, tetrahydroquinoline derivatives, cyclic peptides, para-xylyl-enediamine-based derivatives, isothiourea derivatives and POL6326, POL5551, CCTE-9908 and TG-0054 and optionally, the CXCR4 antagonist is AMD3100.

77. A method according to any one of claims 59 to 76 wherein the antagonist and the CXCR4 antagonist or an active portion thereof is administered in the absence of G-CSF.

78. A method according to any one of claims 59 to 77 wherein the α₉ integrin antagonist and the CXCR4 antagonist or an active portion thereof is administered intravenously, intradermally, subcutaneously, intramuscularly, transdermally, intraperitoneally or transmucosally; optionally the antagonist is administered intravenously or subcutaneously.

79. A method according to any one of claims 59 to 78 wherein the as integrin antagonist and the CXCR4 antagonist or an active portion thereof is administered simultaneously, consecutively or in combination with an α₄ integrin antagonist.

80. A method according to any one of claims 59 to 79 wherein the haematological disorder is selected from the group including immunosuppression, chronic illness, traumatic injury, degenerative disease, infection, or combinations thereof; a disease or condition of the skin, digestive system, nervous system, lymph system, cardiovascular system, endocrine system, or combinations thereof; osteoporosis, Alzheimer's disease, cardiac infarction, Parkinson's disease, traumatic brain injury, multiple sclerosis, cirrhosis of the liver, or combinations thereof; neuroblastoma, myelodysplasia, myelofibrosis, breast cancer, renal cell carcinoma, or multiple myeloma; haematopoietic neoplastic disorder; autoimmune disease; or non-malignant disorder.

81. A method according to claim 58 to 80 wherein the haematological disorder is acute lymphoblastic leukemia (ALL) selected form the group including B-lineage ALL and T-lineage ALL, chronic lymphocytic leukemia (CLL), prolymphocytic leukemia (PLL), hairy cell leukemia (HLL) and Waldenstrom's macroglobulinemia (WM).

82. A method of transplanting HSC into a patient, said method comprising
administering an α₉ integrin antagonist and a CXCR4 antagonist or an active portion thereof to a subject to dislodge HSC from a BM stem cell binding ligand;
releasing and mobilizing the HSC from the BM to the PB;
harvesting HSC from the subject; and
transplanting the HSC to the patient.

83. A method according to claim 82 wherein the HSC are long term repopulating HSC/HPC and optionally selected from the group including CD34⁺ cells, CD38+, CD90+, CD133+, CD34⁺CD38⁻ cells, lineage-committed CD34⁻ cells, or CD34⁺CD38⁺ cells; optionally CD34⁺ or CD34⁺CD38⁻ cells.
